(19) United States
(12) Patent Application Publication
(10) Pub. No.: US 2009/0137549 A1
(43) Pub. Date: May 28, 2009

(54) Novel compounds useful for the treatment of degenerative & inflamatory diseases

(76) Inventors: Paul John Edward, Laval (CA); Mark Stuart Chambers, Walden (GB); Christel Jeanne Marie Menet, Mechelen (BE); Stephen Robert Fletcher, Mechelen (BE); Rebecca Elizabeth Jarvis, Walden (GB); Herve Van De Poel, Walden (GB); Alexander Sudau, Leichlingen (DE); Alastair Rae, Saffron Walden (GB); Peter Stanley Thomas, Saffron Walden (GB)

Correspondence Address: KLAUBER & JACKSON, 411 HACKENSACK AVENUE, HACKENSACK, NJ, 07601, US

(21) Appl. No.: 11/983,402

(22) Filed: Nov. 8, 2007

Related U.S. Application Data
(60) Provisional application No. 60/865,031, filed on Nov. 9, 2006.

(51) Int. Cl. A61K 31/397; C07D 487/04; A61P 19/02; C07D 413/14

(52) U.S. Cl. 514/210.18; 544/262; 544/118

(57) ABSTRACT

The present invention relates to compounds that are inhibitors of PDE1A, a phosphodiesterase that is involved in the modulation of the degradation of cartilage, joint degeneration and diseases involving such degradation and/or inflammation.

### RELATED APPLICATIONS

The present application claims the benefit under 35 U.S.C. § 119 of U.S. Provisional Application No. 60/865,031, filed Nov. 9, 2006, the contents of which is hereby incorporated by reference in its entirety.

### BACKGROUND OF THE INVENTION

### Field of Invention

The present invention relates to compounds that are inhibitors of PDE1A, a phosphodiesterase that is involved in the modulation of the degradation of cartilage, joint degeneration and diseases involving such degradation and/or inflammation.

Cartilage is an avascular tissue of which chondrocytes are the main cellular component. The chondrocytes in normal articular cartilage occupy approximately 5% of the tissue volume, while the extra-cellular matrix makes up the remaining 95% of the tissue. The chondrocytes secrete the components of the matrix, mainly proteoglycans and collagens, which in turn supply the chondrocytes with an environment suitable for their survival under mechanical stress. In cartilage, collagen type II, together with the protein collagen type IX, is arranged in solid fibril-like structures, which provide cartilage with great mechanical strength. The proteoglycans can absorb water and are responsible for the resilient and shock absorbing properties of the cartilage.

One of the functional roles of cartilage in the joint is to allow bones to articulate on each other smoothly. Loss of articular cartilage, therefore, causes the bones to rub against each other leading to pain and loss of mobility. The degradation of cartilage can have various causes. In inflammatory arthritis, as in rheumatoid arthritis for example, cartilage degradation is caused by the secretion of proteases (e.g. collagenases) by inflamed tissues (the inflamed synovium for example). Cartilage degradation can also be the result of an injury of the cartilage, due to an accident or surgery, or exaggerated loading or ‘wear and tear’. The ability of cartilage tissue to regenerate after such insults is limited. Chondrocytes in injured cartilage often display reduced cartilage synthesizing (anabolic) activity and/or increased cartilage degrading (catabolic) activity.

The degeneration of cartilage is the hallmark of various diseases, among which rheumatoid arthritis and osteoarthritis are the most prominent.

Rheumatoid arthritis (RA) is a chronic joint degenerative disease, characterized by inflammation and destruction of the joint structures. When the disease is unchecked, it leads to substantial disability and pain due to loss of joint functionality and even premature death. The aim of an RA therapy, therefore, is not to slow down the disease but to attain remission in order to stop the joint destruction. Besides the severity of the disease outcome, the high prevalence of RA (˜0.8% of adults are affected worldwide) means a high socioeconomic impact. (For reviews on RA, we refer to Smolen and Steiner (2003); Lee and Weinblatt (2001); Choy and Panayi (2001); O'Dell (2004) and Firestein (2003)).

Osteoarthritis (also referred to as OA, or wear-and-tear arthritis) is the most common form of arthritis and is characterized by loss of articular cartilage, often associated with hypertrophy of the bone and pain. The disease mainly affects hands and weight-bearing joints such as knees, hips and spines. This process thins the cartilage. When the surface area has disappeared due to the thinning, a grade I osteoarthritis is reached; when the tangential surface area has disappeared, grade II osteoarthritis is reached. There are further levels of degeneration and destruction, which affect the deep and the calcified cartilage layers that border with the subchondral bone. For an extensive review on Osteoarthritis, refer to Wieland et al., 2005.

The clinical manifestations of the development of the osteoarthritis condition include: increased volume of the joint, pain, crepitation and functional disability that, lead to pain and reduced mobility of the joints. When disease further develops, pain at rest emerges. If the condition persists without correction and/or therapy, the joint is destroyed leading to disability. Replacement surgery with total prosthesis is then required.

Therapeutic methods for the correction of the articular cartilage lesions that appear during the osteoarthritic disease have been developed, but so far none of them have been able to mediate the regeneration of articular cartilage in situ and in vivo.

### Reported Developments

Osteoarthritis is difficult to treat. At present, no cure is available and treatment focuses on relieving pain and preventing the affected joint from becoming deformed. Common treatments include the use of non-steroidal anti-inflammatory drugs (NSAID's). Although the dietary supplements as chondroitin and glucosamine sulphate have been advocated as safe and effective options for the treatment of osteoarthritis, a recent clinical trial revealed that both treatments did not reduce pain associated to osteoarthritis. (Clegg et al., 2006). Taken together, no disease modifying osteoarthritic drugs are available.

In severe cases, joint replacement may be necessary. This is especially true for hips and knees. If a joint is extremely painful and cannot be replaced, it may be fused. This procedure stops the pain, but results in the permanent loss of joint function, making walking and bending difficult.

Another possible treatment is the transplantation of cultured autologous chondrocytes. Here chondral cellular material is taken from the patient, sent to a laboratory where it is expanded. The material is then implanted in the damaged tissues to cover the tissue's defects.

Another treatment includes the intra-articular instillation of Hylan G-F 20 (Synvisc, Hyalgan, Artz etc.), a substance that improves temporarily the rheology of the synovial fluid, producing an almost immediate sensation of free movement and a marked reduction of pain.

Other reported methods include application of tendinous, periosteal, fascial, muscular or perichondral grafts; implantation of fibrin or cultured chondrocytes; implantation of synthetic matrices, such as collagen, carbon fiber; administration of electromagnetic fields. All of these have reported minimal and incomplete effects, resulting in a poor quality tissue that can neither support the weighted load nor allow the restoration of an articular function with normal movement.

Stimulation of the anabolic processes, blocking catabolic processes, or a combination of these two, may result in stabilization of the cartilage, and perhaps even reversion of the damage, and therefore prevent further progression of the disease. Various triggers may stimulate anabolic stimulation of chondrocytes. Insulin-like growth factor-I (IGF-I) is the predominant anabolic growth factor in synovial fluid and stimulates the synthesis of both proteoglycans and collagen. It has also been shown that members of the bone morphogenetic protein (BMP) family, notably BMP2, BMP4, BMP6, and BMP7, and members of the human transforming growth factor-b (TGF-b) family can induce chondrocyte anabolic stimulation (Chubinskaya and Kuettner, 2003). A compound has recently been identified that induces anabolic stimulation of chondrocytes (U.S. Pat. No. 6,500,854; EP 1.391211). However, most of these compounds show severe side effects and, consequently, there is a strong need for compounds that stimulate chondrocyte differentiation without these side effects.

Adenosine 3′,5′-cyclic monophosphate (cyclic AMP or cAMP) and guanosine 3′,5′-cyclic monophosphate (cyclic GMP or cGMP) are key second messenger molecules in cells which are synthesized by guanylyl and adenylyl cyclases. These molecules, by playing a role as ‘relay’ on signal transduction pathways, are key in controlling normal and pathological cell responses. Cyclic nucleotide phosphodiesterases (PDE's) are enzymes that hydrolyse cyclic nucleotides and thereby control the cellular levels of these second messenger molecules. Because of their key role in cellular signaling, PDE's are considered new therapeutic targets. Inhibition of PDE4 and PDE5 are accepted approaches for the treatment of asthma/chronic obstructive pulmonary disease and erectile dysfunction, respectively. As such, pharmaceutical industry has recently deployed a lot of efforts to develop PDE4 inhibitors (e.g. Cilomilast) and PDE5 inhibitors (e.g. sildenafil), some of which are marketed.

The diversity of the PDE family of enzymes (11 gene families (PDE1-PDE11) encoding more than 20 different PDE genes) allows a refined control over a variety of cellular processes. For an extensive review on PDE's, we refer to Lugnier, 2006. PDE's classically contain a catalytic domain, which is well conserved among different PDEs. In addition, PDE's contain regulatory domains. The activity of enzymes of the PDE1 subfamily, for example, is regulated by Ca²⁺ and calmodulin as well as by phosphorylation. As such, the PDE1 enzymes are involved in the complex interaction between the Ca²⁺ and cyclic nucleotide second messenger systems. Another feature of PDE1 enzymes is their dual substrate specificity as they have the capacity to hydrolyse both cAMP and cGMP (Zhang et al., 2004).

The generation of transgenic animals represents the best tool for the understanding of the specific physiological role of individual PDEs. In the PDE1 subfamily, a PDE1B knockout mouse has been generated and characterized. PDE1B(−/−) mice showed exaggerated hyperactivity after acute D-methamphetamine administration. PDE1B(−/−) and PDE1B(+/−) mice demonstrated spatial-learning deficits. These results indicate that enhancement of cyclic nucleotide signaling by inactivation of PDE1B-mediated cyclic nucleotide hydrolysis plays a significant role in the central nervous system, especially on the dopaminergic function (Reed et al., 2002). Less is known about the physiological role of the other members of the PDE1 superfamily. A role for PDE1 enzymes (PDE1C in particular) in vascular tone (e.g.; in pulmonary hypertension) has been suggested (Murray et al., 2006). For an extensive review on PDE1 enzymes, we refer to Kakkar et al., 1999 and Goraya and Cooper, 2005.

Several data point to a role of PDEs in chondrocyte biology. First, PDE4 and PDE1 were identified as major PDE activities in chondrocytes (Tenor et al., 2002). The involvement of PDEs in cartilage catabolic events was further evidenced as follows. The IL1 cytokine is responsible for cartilage catabolism by reducing the expression of matrix components, by inducing the expression of collagenases and inducible nitric oxide synthase (iNOS), which mediates the production of nitric oxide (NO). This event appears dependent on PDE activity, as IBMX, PDE5 inhibitor and PDE4 inhibitor treatment of chondrocytes reduced the induction of iNOS expression by IL1 (Geng et al., 1998, Tenor et al., 2002). The ability of PDE inhibitors to reduce iNOS expression appeared dependent on autocrine PGE2 production by the chondrocytes. Taken together, these data suggest a role for PDEs in cartilage catabolic events.

The current therapies are not satisfactory and therefore there remains a need to identify further compounds that may be of use in the treatment of degenerative joint diseases, e.g. osteoarthritis, rheumatoid arthritis and osteoporosis, in particular osteoarthritis. The present invention therefore provides compounds, methods for their manufacture and a pharmaceutical comprising a compound of the invention together with a suitable pharmaceutical carrier. The present invention also provides for the use of a compound of the invention in the preparation of a medicament for the treatment of degenerative joint diseases.

### SUMMARY OF THE INVENTION

The present invention is based on the discovery that inhibitors of PDE1A are useful for the treatment of diseases involving cartilage degradation, joint degradation and/or inflammation, for example osteoarthritis. The present invention also provides methods for the production of these compounds, pharmaceutical compositions comprising these compounds and methods for treating diseases involving cartilage degradation, joint degradation and/or inflammation by administering a compound of the invention.

The compounds of the present invention may be described generally as pyrazolo[3,4-d]pyrimidin-4-ones substituted in the 6-position with a C-linked nitrogen substituted cycloalkylamine.

Accordingly, the present invention relates to compounds having anti-inflammatory properties, according to formula (I):

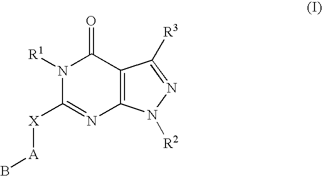

wherein:
A represents a bond, —(CH₂)n-, —CO, —CONR⁴—, CSNR⁴—, —C(═N—CN)NR⁴—, C(═CH—NO₂)NR⁴, —COO—, —SO₂—, or —SO₂NR⁴—, aryl or heteroaryl, optionally substituted with one or more groups selected from halogen, CF₃, C₁-C₆ alkyl, C₁-C₆ alkylcycloalkyl, cycloalkyl, heterocycloalkyl —SO₂R⁴— and C₁-C₆ alkylheterocycloalkyl, where A is linked to X via a nitrogen atom within the X group;
B represents bond, C₁-C₆ alkyl, (CH₂)ₘ-cycloalkyl, —(CH₂)ₘ-heterocycloalkyl, (CH₂)ₘ-aryl or (CH₂)ₘ heteroaryl optionally substituted with one or more groups selected from halogen, CN, CF₃, NR⁴R⁵, NR⁵COR⁴, CONR⁴R⁵, NR⁵SO₂R⁴, SO₂NR⁴R⁵, C₁-C₆ alkyl, (CH₂)ₙ-heterocycloalkyl (optionally substituted by C₁-C₆ alkyl), NO₂, OR⁴, COR⁴, CO₂R⁴, or SO₂R⁴
X represents a carbon-carbon bonded nitrogen-containing heterocycloalkyl group;
R¹ represents H, C₁-C₆ alkyl, (CH₂)ₙ-aryl, cycloalkyl or —C₁-C₆ alkyl-cycloalkyl group, each of which may optionally be substituted with one or more groups selected from halogen, CN, CF₃, NR⁴R⁵, NHCOR⁴, CONH₂, NHSO₂R⁴, SO₂NHR⁴, C₁-C₆ alkyl, C₁-C₆ alkoxy, COR⁴, CO₂R⁴, or SO₂R⁴;
R² represents H, C₁-C₆ alkyl, cycloalkyl, heterocycloalkyl, (CH₂)ₙ-aryl, or a (CH₂)ₙ-heteroaryl group, each of which may optionally be substituted with one or more groups selected from halogen, CN, CF₃, NR⁴R⁵, NHCOR⁴, CONH₂, NHSO₂R⁴, SO₂NHR⁴, SO₂R⁴, C₁-C₆ alkyl, OR⁴, COR⁴, CO₂R⁴, or SO₂R⁴;
R³ represents H, halogen, C₁-C₆ alkyl, cycloalkyl, (CH₂)ₙ-aryl, aryl, or a heteroaryl group, each of which may optionally be substituted with one or more groups selected from halogen, CN, CF₃, NR⁴R⁵, NHCOR⁴, CONH₂, NHSO₂R⁴, SO₂NHR⁴, SO₂R⁴, C₁-C₆ alkyl, C₁-C₆ alkoxy, COR⁴, CO₂R⁴, or SO₂R⁴;
each R⁴ independently represents H, C₁-C₆ alkyl, cycloalkyl, heterocycloalkyl, CF₃ or CHF₂;
R⁵ represents H, C₁-C₆ alkyl, or cycloalkyl; and
each “n” independently represents 0, 1, 2 or 3;
“m” represents 0, 1, 2, 3, 4, 5 or 6;
   or a pharmaceutically acceptable salt, hydrate, solvate or prodrug thereof or isotopic variants thereof, stereoisomers or tautomers thereof.

More particularly, the present invention relates to compounds having anti-inflammatory properties, according to formulae Ia, Ib, Ic, Id, Ie, If, Ig or Ih:

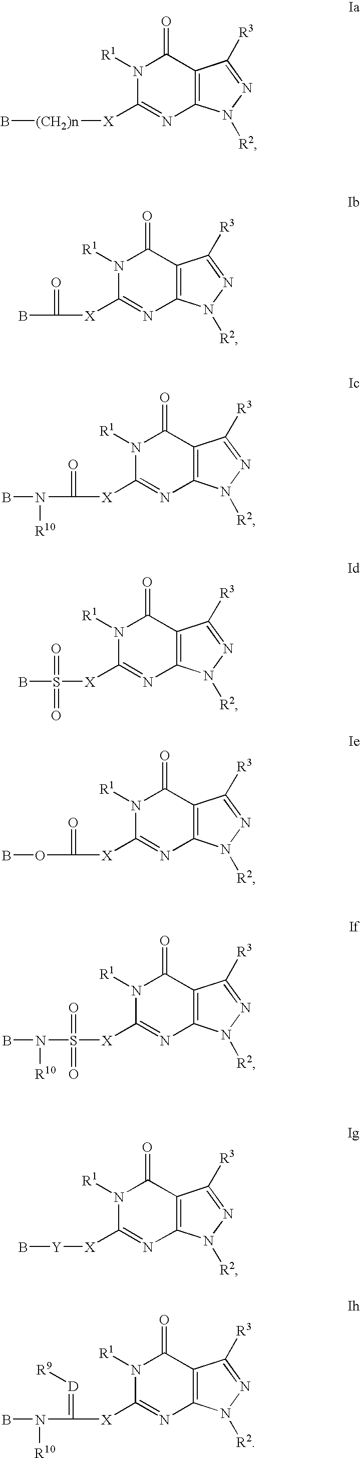

wherein:
X represents a carbon-carbon bonded nitrogen-containing heterocycloalkyl group;
B represents substituted or unsubstituted C₁-C₆ alkyl, or C₁-C₆ haloalkyl;
   or B represents substituted or unsubstituted cycloalkyl, heterocycloalkyl, or cycloalkylalkyl;
   or B represents substituted or unsubstituted aralkyl, aryl or heteroaryl;
   or with respect to a compound according to the formulae Ie or Ig, B further includes H, NO₂, C₁-C₆ alkyl, halo, —CO-aryl, —CO-heteroaryl, —CON(R⁴)-aryl, or CO—N(R⁴)-heteroaryl;
Y represents a bond, substituted or unsubstituted aryl or substituted or unsubstituted heteroaryl;
the group B—(CH₂)n-, B—CO—, B—N(R¹⁰)CO—, B—SO₂—, B—OCO—, B—N(R¹⁰)SO₂—, B—Y— or B—NR¹⁰-D(R⁹)— is linked to X via a nitrogen atom within the X group;
D represents CH or N, with the proviso that when D represents CH, R⁹ represents —NO₂ and when
D represents N, R⁹ represents CN;
R¹ represents H, C₁-C₆ alkyl, (CH₂)ₙ-aryl, cycloalkyl or —C₁-C₆ alkyl-cycloalkyl group, each of which may optionally be substituted with one or more groups selected from halogen, CN, CF₃, NR⁴R⁵, NR⁵COR⁴, CONR⁴R⁵, NR⁵SO₂R⁴, SO₂NR⁵R⁴, C₁-C₆ alkyl, C₁-C₆ alkoxy, COR⁴, CO₂R⁴, or SO₂R⁴;
R² represents H, C₁-C₆ alkyl, cycloalkyl, heterocycloalkyl, cycloalkylalkyl, (CH₂)ₙ-aryl, or a heteroaryl group, each of which may optionally be substituted with one or more groups selected from halogen, CN, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, or SO₂R⁴;
R³ represents H, halogen, C₁-C₆ alkyl, cycloalkyl, (CH₂)n-aryl, aryl, or a heteroaryl group, each of which may optionally be substituted with one or more groups selected from halogen, CN, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, or SO₂R⁴;
R⁴ represents H, C₁-C₆ alkyl, halo C₁-C₆ alkyl, cycloalkyl, or heterocycloalkyl;
R⁵ represents H, C₁-C₆ alkyl, or cycloalkyl;
R⁹ represents CN or NO₂;
R¹⁰ represents H or C₁-C₆ alkyl; and
each “n” independently represents 0, 1, 2 or 3;
   or a pharmaceutically acceptable salt, hydrate, solvate or prodrug thereof or isotopic variants thereof, stereoisomers or tautomers thereof.

In one embodiment, with respect to compounds of formulae Ia-Ih, X is selected from piperidine, pyrrolidine and azetidine. In a particular embodiment X is piperidine or azetidine.

Another aspect of this invention relates to the use of the present compound in a therapeutic method, a pharmaceutical composition, and the manufacture of such composition, useful for the treatment of a disease involving inflammation, and in particular, a disease characteristic of abnormal PDE1A activity. This invention also relates to processes for the preparation of the present compounds.

### BRIEF DESCRIPTION OF THE DRAWINGS

FIG. 1. Shows the mechanism of the primary screening assay using the cAMP dynamic htrf kit from Cisbio.

### DETAILED DESCRIPTION

### Definitions

The following terms are intended to have the meanings presented therewith below and are useful in understanding the description and intended scope of the present invention.

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope. By way of non-limiting example, such substituents may include e.g. halo (such as fluoro, chloro, bromo), —CN, —CF₃, —OH, —OCF₃, C₁-C₆ alkyl, C₂-C₆ alkenyl, C₃-C₆ alkynyl, C₁-C₆ alkoxy, aryl and di-C₁-C₆ alkylamino. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

‘Alkoxy’ means alkyl-O—. Exemplary alkoxy includes methoxy, ethoxy, n-propoxy, i-propoxy, n-butoxy, and heptoxy. Preferred alkoxy groups are lower alkoxy, i.e. with between 1 and 6 carbon atoms.

‘Alkyl’ means straight or branched aliphatic hydrocarbon having 1 to about 20 carbon atoms. In particular, alkyl has 1 to about 12 carbon atoms. A further particular group is lower alkyl which has 1 to 6 carbon atoms. Further particular groups are groups such as methyl, ethyl and propyl. Branched means that one or more lower alkyl groups such as methyl, ethyl or propyl is attached to a linear alkyl chain. The term C₁-C₆ alkyl includes both branched and straight chain groups, exemplary straight chain groups include ethyl, propyl, butyl as listed above, exemplary branched chain groups include isopropyl, isoamyl.

‘Alkyl amino’ means alkyl-NH—. Preferred alkyl amino is (C₁-C₆)-alkyl amino. Exemplary alkyl amino includes methylamino and ethylamino.

‘Amino lower alkanoyl’ means NH₂—R—CO—, where R is lower alkylene. Preferred groups include aminoethanoyl and aminoacetyl.

‘Aralkyl’ or ‘arylalkyl’ refers to a radical in which an aryl group is substituted for a hydrogen atom of an alkyl group.

‘Acyl’ refers to a radical —C(O)R²⁰, where R²⁰ is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cyclohexylcarbonyl, cyclohexylmethylcarbonyl, benzoyl, benzylcarbonyl and the like.

‘Aryl’ refers to a monovalent aromatic hydrocarbon group derived by the removal of one hydrogen atom from a single carbon atom of a parent aromatic ring system. Aryl groups may be monocyclic or a bicyclic fused-ring structure where at least one of the rings is an aromatic ring structure that preferentially contains 6 carbons. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta 2,4 diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms. Particularly, the aryl group may contain 6 carbon atoms, exemplary aryl groups include phenyl and indan-1-one.

‘Substituted Aryl’ includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

‘Bicycloaryl’ refers to a monovalent aromatic hydrocarbon group derived by the removal of one hydrogen atom from a single carbon atom of a parent bicycloaromatic ring system. Typical bicycloaryl groups include, but are not limited to, groups derived from indane, indene, naphthalene, tetrahydronaphthalene, and the like. Particularly, an aryl group comprises from 8 to 11 carbon atoms.

‘Carbamoyl’ refers to the radical —C(O)N(R⁴²)₂ where each R⁴² group is independently hydrogen, alkyl, cycloalkyl or aryl, as defined herein, which may be optionally substituted as defined herein. In a specific embodiment, the term “carbamoyl” refers to —C(O)—NH₂. In an alternative embodiment ‘carbamoyl lower alkyl’ means the radical NH₂CO-lower alkyl-. Preferred carbamoyl lower alkyl groups include carbamoylethyl and carbamoylmethyl.

‘Carboxy lower alkyl ester’ means a lower alkyl ester of a carboxy radical, —COO— group.

‘Compounds of the present invention’, and equivalent expressions, are meant to embrace the compounds as hereinbefore described, in particular compounds according to Formula (I) and/or Formulae Ia-Ih, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

‘Expression’ means endogenous or exogenous expression.

‘Halo’ or ‘halogen’ means fluoro, chloro, bromo, or iodo.

‘Hydrogen’ means in the context of a substituent that —H is present at the compound position and also includes its isotope, deuterium.

‘Lower alkanoyl amino’ means an amino group with an organic functional group R—CO—, where R represents a lower alkyl group.

‘Lower alkyl’ means 1 to about 6 carbon atoms in a linear alkyl chain that may be straight or branched.

‘Lower alkoxy’ means 1 to about 6 carbon atoms in a linear alkyl chain that may be straight or branched, and that is bonded by an oxygen atom.

‘Lower alkyl sulphonamide’ refers to a lower alkyl amide of sulphonamide of the formula —SO2NR*R*, where R* is hydrogen or lower alkyl, and at least one R* is lower alkyl.

‘Sulphonamide’ refers to a group of compounds containing the chemical group —SO₂NH₂.

‘Cycloalkyl’ refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like. Particular cycloalkyl groups have between 4 and 7 carbon ring members for example cyclopropyl, cyclobutyl, cyclopentyl, cyclohexyl and cycloheptyl.

‘Substituted cycloalkyl’ includes those groups recited in the definition of ‘substituted’ herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Substituted’ refers to a group in which one or more hydrogen atoms are each independently replaced with the same or different substituent(s). Typical substituents include, but are not limited to, —X, —R⁴⁶, —O, ═O, —OR⁴⁶, —SR⁴⁶, —S, ═S, NR⁴⁶R⁴⁷, ═NR⁴⁶, —CX₃, —CF₃, —CN, —OCN, —SCN, —NO, —NO₂, ═N₂, —N₃, —S(O)₂O, S(O)₂OH, —S(O)₂R⁴⁶, —OS(O₂)O—, —OS(O)₂R⁴⁶, —P(O)(O—)₂, —P(O)(OR⁴⁶)(O—), OP(O)(OR⁴⁶)(OR⁴⁷), —C(O)R⁴⁶, C(S)R⁴⁶, —C(O)OR⁴⁶, —C(O)NR⁴⁶R⁴⁷, —C(O)O, C(S)OR⁴⁶, —NR⁴⁸C(O)NR⁴⁶R⁴⁷, —NR⁴⁸C(S)NR⁴⁶R⁴⁷, —NR⁴⁹C(NR⁴⁸)NR⁴⁶R⁴⁷ and C(NR⁴¹)NR⁴⁶R⁴⁷, where each X is independently a halogen; each R⁴⁶, R⁴⁷, R⁴⁸ and R⁴⁹ are independently hydrogen, alkyl, substituted alkyl, aryl, substituted aryl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁰R⁵¹, —C(O)R⁵⁰ or S(O)₂R⁵⁰ or optionally R⁵⁰ and R⁵¹ together with the atom to which they are both attached form a cycloheteroalkyl or substituted cycloheteroalkyl ring; and R⁵⁰ and R⁵¹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

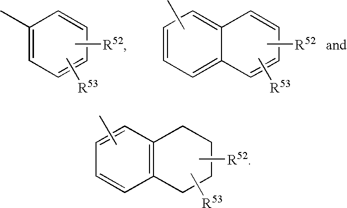

In these formulae one of R⁵² and R⁵³ may be hydrogen and at least one of R⁵² and R⁵³ is each independently selected from alkyl, alkenyl, alkynyl, cycloheteroalkyl, alkanoyl, alkoxy, aryloxy, heteroaryloxy, alkylamino, arylamino, heteroarylamino, NR⁵⁴COR⁵⁵, NR⁵⁴SOR⁵⁵, NR⁵⁴SO₂R⁵⁷, COO-alkyl, COO-aryl, CONR⁵⁴R⁵⁵, CONR⁵⁴OR⁵⁵, NR⁵⁴R⁵⁵, S2NR⁵⁴R⁵⁵, S-alkyl, S-alkyl, SO-alkyl, SO₂-alkyl, S-aryl, SO-aryl, SO₂-aryl; or R⁵² and R⁵³ may be joined to form a cyclic ring (saturated or unsaturated) from 5 to 8 atoms, optionally containing one or more heteroatoms selected from the group N, O or S. R⁵⁴, R⁵⁵, and R⁵⁶ are independently hydrogen, alkyl, alkenyl, alkynyl, perfluoroalkyl, cycloalkyl, cycloheteroalkyl, aryl, substituted aryl, heteroaryl, substituted or hetero alkyl or the like.

‘Hetero’ when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, heterocycloalkenyl, and the like having from 1 to 5, and especially from 1 to 3 heteroatoms.

‘Heteroaryl’ refers to a monovalent heteroaromatic group derived by the removal of one hydrogen atom from a single atom of a parent heteroaromatic ring system. The heteroaryl group may be a monocyclic group (in which case it will typically be a 5 to 7, more typically a 5 or 6 membered ring), alternatively the heteroaryl group may be a bicycloheteroaryl group in particular a fused ring system comprising 2 fused 5-membered rings, a fused 5 and 6 membered ring or two fused 6 membered rings, where the heteroaryl group comprises fused rings at least one of said rings should contain a heteroatom and at least one said rings should be aromatic (both requirements may or may not be fulfilled in the same ring). The heteroaryl group can be, for example, a five membered or six membered monocyclic ring which may contain up to about four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-15 membered heteroaryl, with 5-10 membered heteroaryl being particularly preferred. Particular heteroaryl groups are those derived from thiophene, pyrrole, benzothiophene, benzofuran, indole, pyridine, quinoline, imidazole, oxazole and pyrazine. Particularly, examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups. Particularly, examples of six membered heteroaryl groups include but are not limited to pyridine, pyrazine, pyridazine, pyrimidine and triazine.

Examples of representative heteroaryls include the following:

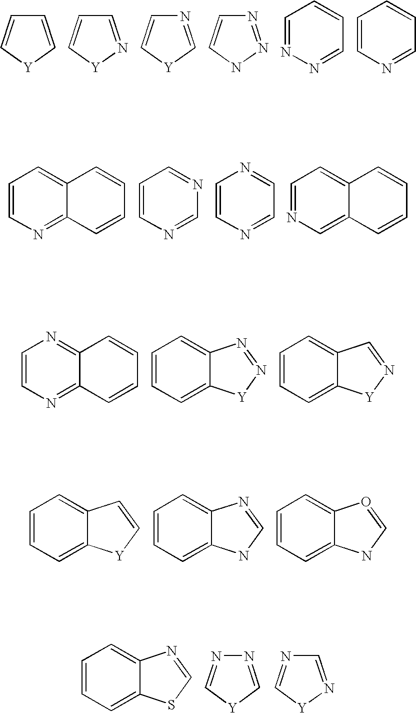

wherein each Y is selected from carbonyl, N, NR⁵⁸, O, and S; and R⁵⁸ is independently hydrogen, alkyl, cycloalkyl, cycloheteroalkyl, aryl, heteroaryl, heteroalkyl or the like.

‘Bicycloheteroaryl’ refers to a monovalent bicycloheteroaromatic group derived by the removal of one hydrogen atom from a single atom of a parent bicycloheteroaromatic ring system. Typical bicycloheteroaryl groups include, but are not limited to, groups derived from benzofuran, benzimidazole, benzindazole, benzdioxane, chromene, chromane, cinnoline, phthalazine, indole, indoline, indolizine, isobenzofuran, isochromene, isoindole, isoindoline, isoquinoline, benzothiazole, benzoxazole, naphthyridine, benzoxadiazole, pteridine, purine, benzopyran, benzpyrazine, pyridopyrimidine, quinazoline, quinoline, quinolizine, quinoxaline, benzomorphan, tetrahydroisoquinoline, tetrahydroquinoline, and the like. Preferably, the bicycloheteroaryl group is between 9-11 membered bicycloheteroaryl, with 5-10 membered heteroaryl being particularly preferred. Particular bicycloheteroaryl groups are those derived from benzothiophene, benzofuran, benzothiazole, indole, quinoline, isoquinoline, benzimidazole, benzoxazole, benzo[1,3]dioxalyl and benzodioxane.

‘Heterocycloalkyl’ refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples:

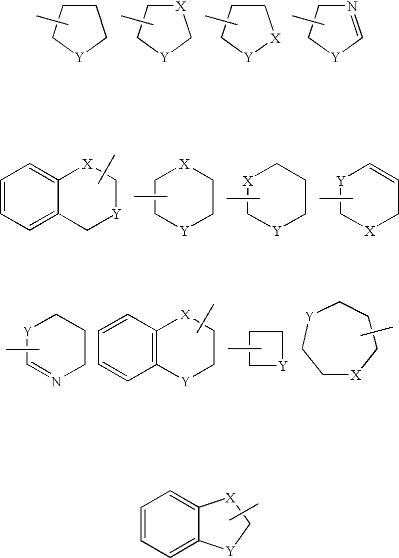

wherein each X is selected from CR⁵⁸₂, NR⁵⁸, O and S; and each Y is selected from NR⁵⁸, O and S; and R⁵⁸ is independently hydrogen, alkyl, cycloalkyl, cycloheteroalkyl, aryl, heteroaryl, heteroalkyl or the like. These cycloheteroalkyl rings may be optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives.

“Nitrogen-Containing Heterocycloalkyl” group means a 4 to 7 membered non-aromatic cyclic group containing at least one Nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

Examples of representative aryl having hetero atoms containing substitution include the following:

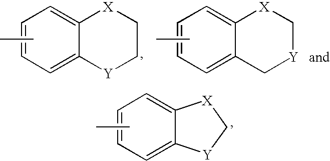

wherein each X is selected from CR⁵⁸₂, NR⁵⁸, O and S; and each Y is selected from carbonyl, NR⁵⁸, O and S; and R⁵⁸ is independently hydrogen, alkyl, cycloalkyl, cycloheteroalkyl, aryl, heteroaryl, heteroalkyl or the like.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

‘Sulfonyl’ refers to the group —SO₂R⁶³. In particular embodiments, R⁶³ is selected from H, lower alkyl, alkyl, aryl and heteroaryl.

‘Pharmaceutically acceptable’ means approved by a regulatory agency of the Federal or a state government or listed in the U.S. Pharmacopoeia or other generally recognized pharmacopoeia for use in animals, and more particularly in humans.

‘Pharmaceutically acceptable vehicle’ refers to a diluent, adjuvant, excipient or carrier with which a compound of the invention is administered.

‘Pharmaceutically acceptable salt’ refers to the non-toxic, inorganic and organic acid addition salts, and base addition salts, of compounds of the present invention, in particular they are pharmaceutically acceptable and possess the desired pharmacological activity of the parent compound. These salts can be prepared in situ during the final isolation and purification of compounds useful in the present invention. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

‘Solvate’ means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. Conventional solvents include water, ethanol, acetic acid and the like, therefore, representative solvates include hydrates, ethanolates and methanolates.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

‘Isotopic variant’ refers to a compound that contains unnatural proportions of isotopes at one or more of the atoms that constitute such compound. For example, an “isotopic variant” of a compound can contain one or more non-radioactive isotopes, such as for example, deuterium (²H or D), carbon 13 (¹³C), nitrogen-15 (¹⁵N), or the like. It will be understood that, in a compound where such isotopic substitution is made, the following atoms, where present, may vary, so that for example, any hydrogen may be 2H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

‘Subject’ includes humans. The terms ‘human’, ‘patient’ and ‘subject’ are used interchangeably herein.

‘Prophylaxis’ means a measure taken for the prevention of a disease.

‘Preventing’ or ‘prevention’ refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

‘Therapeutically effective amount’ means that amount of a drug or pharmaceutical agent that will elicit the biological or medical response of a subject that is being sought by a medical doctor or other clinician. The “therapeutically effective amount” can vary depending on the compound, the disease and its severity, and the age, weight, etc., of the subject to be treated.

### The Compounds

The compounds of the present invention may be described generally as pyrazolo[3,4-d]pyrimidin-4-ones substituted in the 6-position with a C-linked nitrogen substituted cycloalkylamine.

Accordingly, the present invention relates to compounds having anti-inflammatory properties, according to formula (I):

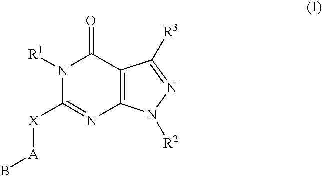

wherein:
A represents a bond, —(CH₂)n-, —CO, —CONR⁴—, CSNR⁴—, —C(═N—CN)NR⁴—, C(═CH—NO₂)NR⁴, —COO—, —SO₂—, or —SO₂NR⁴—, aryl or heteroaryl, optionally substituted with one or more groups selected from halogen, CF₃, C₁-C₆ alkyl, C₁-C₆ alkylcycloalkyl, cycloalkyl, heterocycloalkyl —SO₂R⁴— and C₁-C₆ alkylheterocycloalkyl, where A is linked to X via a nitrogen atom within the X group;
B represents H, C₁-C₆ alkyl, (CH₂)ₘ-cycloalkyl, —(CH₂)ₘ-heterocycloalkyl, (CH₂)ₘ-aryl or (CH₂)ₘ heteroaryl optionally substituted with one or more groups selected from halogen, CN, CF₃, NR⁴R⁵, NR⁵COR⁴, CONR⁴R⁵, NR⁵SO₂R⁴, SO₂NR⁴R⁵, C₁-C₆ alkyl, (CH₂)ₙ-heterocycloalkyl (optionally substituted by C₁-C₆ alkyl), NO₂, OR⁴, COR⁴, CO₂R⁴, or SO₂R⁴
X represents a carbon-carbon bonded nitrogen-containing heterocycloalkyl group;
R¹ represents H, C₁-C₆ alkyl, (CH₂)ₙ-aryl, cycloalkyl or —C₁-C₆ alkyl-cycloalkyl group, each of which may optionally be substituted with one or more groups selected from halogen, CN, CF₃, NR⁴R⁵, NHCOR⁴, CONH₂, NHSO₂R⁴, SO₂NHR⁴, C₁-C₆ alkyl, C₁-C₆ alkoxy, COR⁴, CO₂R⁴, or SO₂R⁴;
R² represents H, C₁-C₆ alkyl, cycloalkyl, heterocycloalkyl, (CH₂)ₙ-aryl, or a (CH₂)ₙ-heteroaryl group, each of which may optionally be substituted with one or more groups selected from halogen, CN, CF₃, NR⁴R⁵, NHCOR⁴, CONH₂, NHSO₂R⁴, SO₂NHR⁴, SO₂R⁴, C₁-C₆ alkyl, OR⁴, COR⁴, CO₂R⁴, or SO₂R⁴;
R³ represents H, halogen, C₁-C₆ alkyl, cycloalkyl, (CH₂)ₙ-aryl, aryl, or a heteroaryl group, each of which may optionally be substituted with one or more groups selected from halogen, CN, CF₃, NR⁴R⁵, NHCOR⁴, CONH₂, NHSO₂R⁴, SO₂NHR⁴, SO₂R⁴, C₁-C₆ alkyl, C₁-C₆ alkoxy, COR⁴, CO₂R⁴, or SO₂R⁴;
each R⁴ independently represents H, C₁-C₆ alkyl, cycloalkyl, heterocycloalkyl, CF₃ or CHF₂;
R⁵ represents H, C₁-C₆ alkyl, or cycloalkyl; and
each “n” independently represents 0, 1, 2 or 3;
“m” represents 0, 1, 2, 3, 4, 5, or 6;
   or a pharmaceutically acceptable salt, hydrate, solvate or prodrug thereof or isotopic variants thereof, stereoisomers or tautomers thereof.

More particularly, the present invention relates to compounds having anti-inflammatory properties, according to formulae Ia, Ib, Ic, Id, Ie, If, Ig, or Ih:

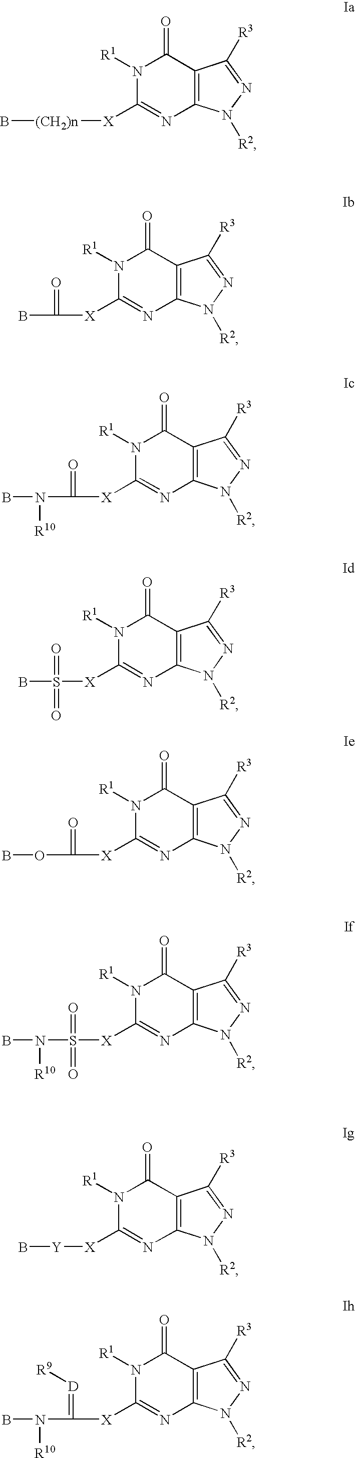

wherein:
X represents a carbon-carbon bonded nitrogen-containing heterocycloalkyl group;
B represents substituted or unsubstituted C₁-C₆ alkyl, or C₁-C₆ haloalkyl;
   or B represents substituted or unsubstituted cycloalkyl, heterocycloalkyl, cycloalkylalkyl or heterocycloalkylalkyl;
   or B represents substituted or unsubstituted aralkyl, aryl or heteroaryl;
   or with respect to a compound according to the formulae Ia or Ig, B further includes H, NO₂, C₁-C₆ alkyl, halo, —CO-aryl, —CO-heteroaryl, —CON(R⁴)-aryl, or CO—N(R⁴)-heteroaryl;
Y represents a bond, substituted or unsubstituted aryl or substituted or unsubstituted heteroaryl;
the group B—(CH₂)n—, B—CO—, B—N(R¹⁰)CO—, B—SO₂—, B—OCO—, B—N(R¹⁰)SO₂—, B—Y— or B—NR¹⁰-D(R⁹)— is linked to X via a nitrogen atom within the X group;
D represents CH or N, with the proviso that when D represents CH, R⁹ represents —NO₂ and when D represents N, R⁹ represents CN;
R¹ represents H, C₁-C₆ alkyl, (CH₂)n-aryl, cycloalkyl or —C₁-C₆ alkyl-cycloalkyl group, each of which may optionally be substituted with one or more groups selected from halogen, CN, CF₃, NR⁴R⁵, NR⁵COR⁴, CONR⁴R⁵, NR⁵SO₂R⁴, SO₂NR⁵R⁴, C₁-C₆ alkyl, C₁-C₆ alkoxy, COR⁴, CO₂R⁴, or SO₂R⁴;
R² represents H, C₁-C₆ alkyl, cycloalkyl, heterocycloalkyl, cycloalkylalkyl, (CH₂)n-aryl, or a heteroaryl group, each of which may optionally be substituted with one or more groups selected from halogen, CN, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, or SO₂R⁴;
R³ represents H, halogen, C₁-C₆ alkyl, cycloalkyl, (CH₂)ₙ-aryl, aryl, or a heteroaryl group, each of which may optionally be substituted with one or more groups selected from halogen, CN, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, or SO₂R⁴;
R⁴ represents H, C₁-C₆ alkyl, halo C₁-C₆ alkyl, cycloalkyl, or heterocycloalkyl;
R⁵ represents H, C₁-C₆ alkyl, or cycloalkyl;
R⁹ represents CN or NO₂;
R¹⁰ represents H or C₁-C₆ alkyl; and
each “n” independently represents 0, 1, 2 or 3;
   or a pharmaceutically acceptable salt, hydrate, solvate or prodrug thereof or isotopic variants thereof, stereoisomers or tautomers thereof.

In one particular embodiment, the compound is according to formula Ia.

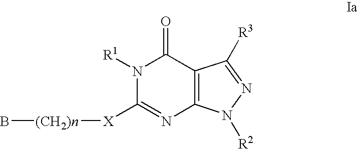

and wherein B, X, R¹, R², R³ and n are as described above.

In another particular embodiment, the compound is according to formula Ib.

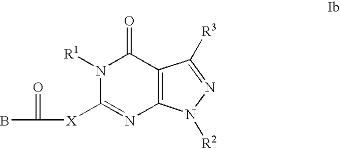

and wherein B, X, R¹, R², and R³ are as described above.

In yet another particular embodiment, the compound is according to formula Ic.

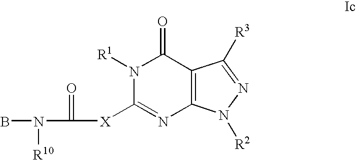

and wherein B, X, R¹, R², R³ and R¹⁰ are as described above.

In yet another particular embodiment, the compound is according to formula Id.

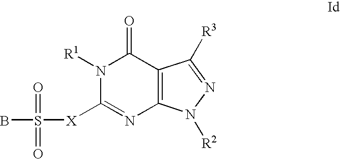

and wherein B, X, R¹, R², and R³ are as described above.

In yet another particular embodiment, the compound is according to formula Ie.

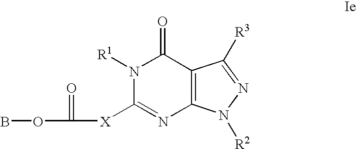

and wherein B, X, R¹, R², and R³ are as described above.

In yet another particular embodiment, the compound is according to formula If.

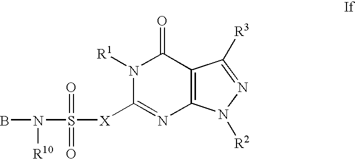

and wherein B, X, R¹, R², R³ and R¹⁰ are as described above.

In yet another particular embodiment, the compound is according to formula Ig.

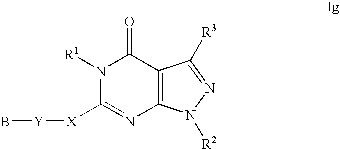

and wherein B, X, Y, R¹, R², and R³ are as described above.

In yet another particular embodiment, the compound is according to formula Ih.

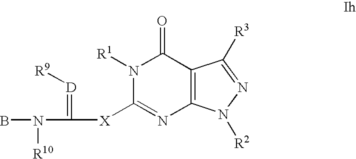

and wherein B, X, D, R¹, R², R³, R⁹ and R¹¹ are as described above

In one embodiment, with respect to compounds of formulae Ia-Ih, B is substituted aralkyl, aryl or heteroaryl and the substitution is selected from halogen, CN, CF₃, NR⁴R⁵, NHCOR⁴, CONH₂, NHSO₂R⁴, SO₂NHR⁴, C₁-C₆ alkyl, C₁-C₆ haloalkyl, C₁-C₆ alkoxy, C₁-C₆ haloalkoxy, COR⁴, CO₂R⁴, and SO₂R⁴.

In another embodiment, with respect to compounds of formulae Ia-Ih, B is substituted aralkyl, aryl or heteroaryl and the substitution is selected from substituted or unsubstituted aryl, heterocycloalkyl and heterocycloalkylalkyl.

In one embodiment, with respect to compounds of formulae Ia-Ih, X is selected from piperidine, pyrrolidine and azetidine. In a particular embodiment X is piperidine or azetidine.

In one embodiment, with respect to compounds of formula Ia, n is 0 and B is other than NO₂ or halo.

In one embodiment, with respect to compounds of formula Ia, n is 0 and B is substituted or unsubstituted cycloalkyl, heterocycloalkyl, cycloalkylalkyl or heterocycloalkylalkyl; In another embodiment, with respect to compounds of formula Ia, n is 0 and B is substituted or unsubstituted aralkyl, aryl or heteroaryl.

In another embodiment, with respect to compounds of formula Ia, n is 0 and B is C₁-C₆ alkyl, —CO-aryl, —CO-heteroaryl, —CON(R⁴)-aryl, or —CO—N(R⁴)-heteroaryl.

In one embodiment, with respect to compounds of formula Ig, Y is a bond, and B is other than NO₂ or halo.

In another embodiment, with respect to compounds of formulae Ia-Ih, the compound is according to formulae IIa, IIb, IIc, IId, IIe, IIf, IIg, or IIh:

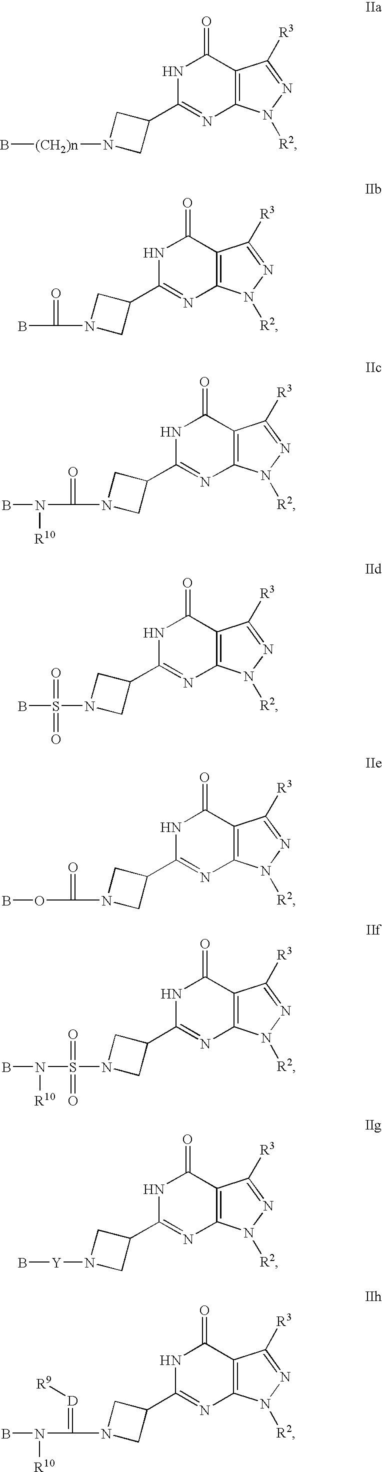

wherein B, Y, D, R², R³ and R⁹ are as defined for formulae Ia-Ih; R¹⁰ is H or C₁-C₆ alkyl; or a pharmaceutically acceptable salt, hydrate, solvate or prodrug thereof or isotopic variants thereof, stereoisomers or tautomers thereof. In one embodiment R¹⁰ is H or Me. In a particular embodiment R¹⁰ is H.

In another embodiment, with respect to compounds of formulae Ia-Ih, the compound is according to formulae IIIa, IIIb, IIIc, IIId, IIIe, IIIf, IIIg, or IIIh:

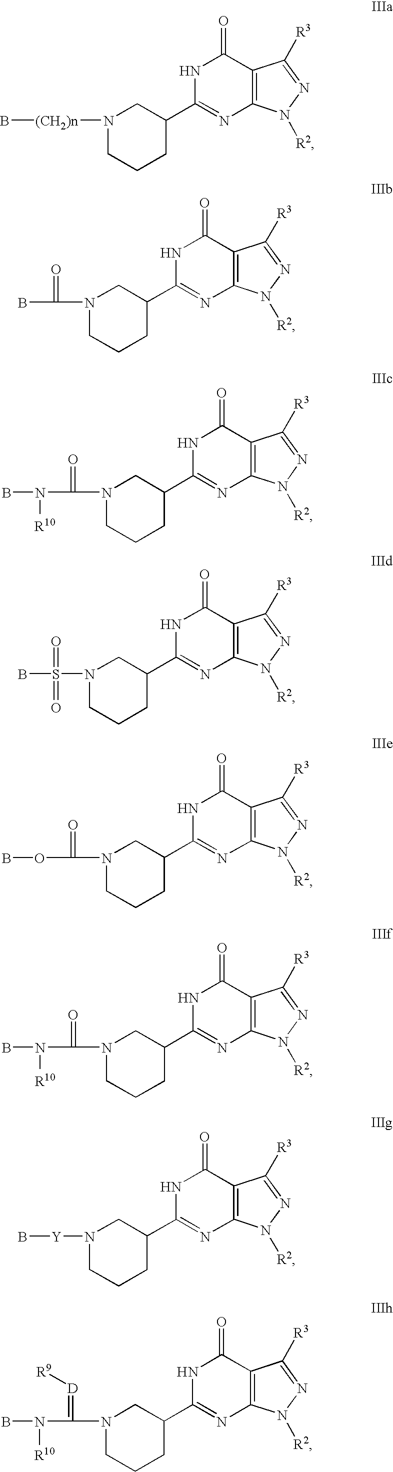

wherein B, Y, D, R², R³ and R⁹ are as in claim 1; R¹⁰ is H or Me; or a pharmaceutically acceptable salt, hydrate, solvate or prodrug thereof or isotopic variants thereof, stereoisomers or tautomers thereof. In one embodiment R¹⁰ is H or Me. In a particular embodiment R¹⁰ is H.

In one embodiment, with respect to compounds of formulae Ia-IIIg, R² is C₁-C₆ alkyl, cycloalkyl, aryl, heteroaryl or heterocycloalkyl.

In another embodiment, with respect to compounds of formulae Ia-IIIg, R² is Me, i-Pr, t-Bu, cyclohexyl, cyclopentyl, cyclobutyl, phenyl, 4-fluorophenyl, pyridyl or pyrrolidinyl. In a particular embodiment, R² is t-Bu, cyclohexyl cyclobutyl, phenyl or 4-fluorophenyl.

In one embodiment, with respect to compounds of formulae Ia-IIIg, R³ is H or C₁-C₆ alkyl.

In another embodiment, with respect to compounds of formulae Ia-IIIg, R³ is H or Me. In a particular embodiment R³ is H.

In one embodiment, with respect to compounds of formulae IIa-IIIg, B is C₁-C₆ alkyl, C₁-C₆ haloalkyl, cycloalkyl, cycloalkylalkyl, heterocycloalkylalkyl, aralkyl, heteroarylalkyl, or heterocycloalkyl.

In another embodiment, with respect to compounds of formulae IIa-IIIg, B is n-Bu, t-Bu, Me, CF₃, 2,2-dimethylpropyl, 3,3,3-trifluoropropyl, cyclohexyl, cyclopentyl, cyclobutyl, cyclopropyl, cyclohexylmethyl, benzyl, 4-fluorobenzyl, 3,4-dichlorobenzyl, alpha-methylbenzyl, piperidinyl, or tetrahydropyranyl.

In another embodiment, with respect to compounds of formulae IIa-IIIg, B is unsubstituted or substituted aryl.

In another embodiment, with respect to compounds of formulae IIa-IIIf, B is phenyl unsubstituted or substituted with one or more groups selected from halogen, CN, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, and SO₂R⁴.

In another embodiment, with respect to compounds of formulae IIa-IIIg, B is phenyl substituted with one or more groups selected from Me, Et, i-Pr, n-Bu, t-Bu, F, Cl, CF₃, OMe, OEt, OCF₃, OCHF₂, CN, NO₂, CO₂Me, NHAc, NH₂, NMe₂, COMe, NHSO₂Me, NHSO₂Et, and NHSO₂—(CH₂)₄-Me.

In another embodiment, with respect to compounds of formulae IIa-IIIh, B is phenyl substituted with substituted or unsubstituted aryl, cycloalkyl, heterocycloalkyl or heteroaryl.

In another embodiment, with respect to compounds of formulae IIa-IIIg, B is phenyl substituted with piperazin-1-yl, N-methylpiperazin-1-yl, N-isopropylpiperazin-1-yl, morpholin-1-yl, piperidin-1-yl, pyrrolidin-1-yl, or morpholin-1-ylmethyl.

In another embodiment, with respect to compounds of formulae IIa-IIIg, B is substituted or unsubstituted heteroaryl.

In another embodiment, with respect to compounds of formulae IIa-IIIg, B is benzo[1,3]dioxalyl, benzimidazol-2-yl, benzthiazol-2-yl, benzoxazol-2-yl, oxazol-2-yl, oxadiazolyl, thiazol-2-yl, imidazol-2-yl, or tetrazolyl; unsubstituted or substituted with one or more groups selected from alkyl, haloalkyl, halo, heterocycloalkyl, heterocycloalkylalkyl, heterocycloalkylphenyl, aryl and heteroaryl.

In one embodiment, with respect to compounds of formulae Ia or IIIa, n is 0 and B is other than NO₂ or halo.

In one embodiment, with respect to compounds of formulae Ia or IIIa, n is 0 and B is substituted or unsubstituted cycloalkyl, heterocycloalkyl, cycloalkylalkyl or heterocycloalkylalkyl;

In another embodiment, with respect to compounds of formulae Ia or IIIa, n is 0 and B is substituted or unsubstituted aralkyl, aryl or heteroaryl.

In another embodiment, with respect to compounds of formulae Ia or IIIa, n is 0 and B is C₁-C₆ alkyl, —CO-aryl, —CO-heteroaryl, —CON(R⁴)-aryl, or —CO—N(R⁴)-heteroaryl.

In one embodiment, with respect to compounds of formulae IIg or IIIg, Y is a bond, and

B is other than NO₂ or halo.

In another embodiment, with respect to compounds of formulae Ig, IIIg, and IIIg, the group B—Y— is selected from

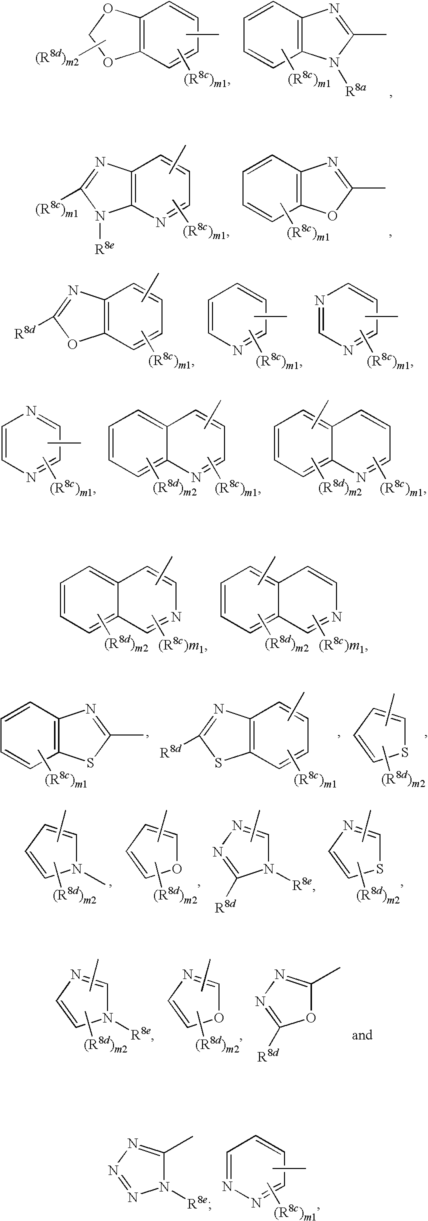

wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴, and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e}, is selected from H, C₁-C₆ alkyl, and halo C₁-C₆ alkyl; or
   R^{8e} is selected from heterocycloalkylalkyl, cycloalkyl, heterocycloalkyl, heterocycloalkylphenyl, aryl or heteroaryl; and each of subscript m1 and m2 is independently selected from 0, 1 and 2.

In one embodiment, with respect to compounds of formulae Ig, IIg and IIIg, the group B—Y— is as described above and each of R^{8c} or R^{8d} is independently selected from H, Me, t-Bu, F, Cl, CF₃; and each of subscript m1 and m2 is independently selected from 1 and 2.

In another embodiment, with respect to compounds of formulae Ig, IIg and IIIg, the group B—Y— is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae Ig, IIg and IIIg, the group B—Y— is as described above and each of R^{8c} or R^{8d} is independently selected from H, Ph, morpholinylmethyl, piperidinylmethyl, piperazinylmethyl, N-Me-piperazinylmethyl, N-i-Pr-piperazinylmethyl; and each of subscript m1 and m2 is independently selected from 1 and 2.

In another embodiment, with respect to compounds of formulae Ig, IIg and IIIg, the group B—Y— is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In yet another embodiment, with respect to compounds of formulae Ig, IIg, and IIIg, the group B—Y— is selected from

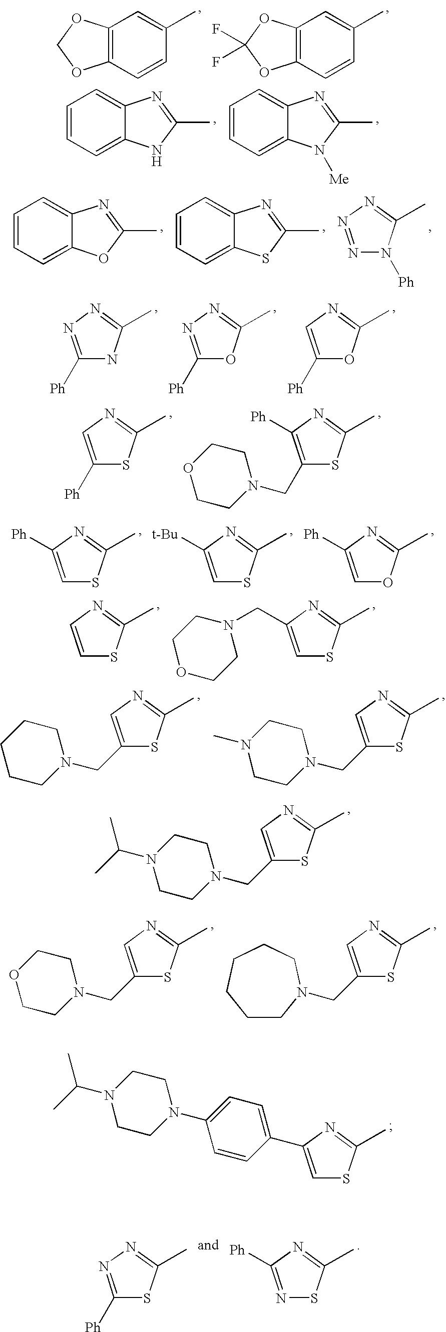

In one embodiment, the compound is according to formula

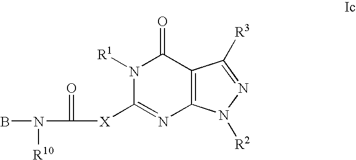

and wherein B, X, R¹, R², R³ and R¹⁰ are as described for formulae Ia-Ih.

In one embodiment, with respect to compounds of formula Ic, R¹⁰ is H.

In one embodiment, with respect to compounds of formula Ic, the compound is according to formulae IVa, IVb, IVc or IVd:

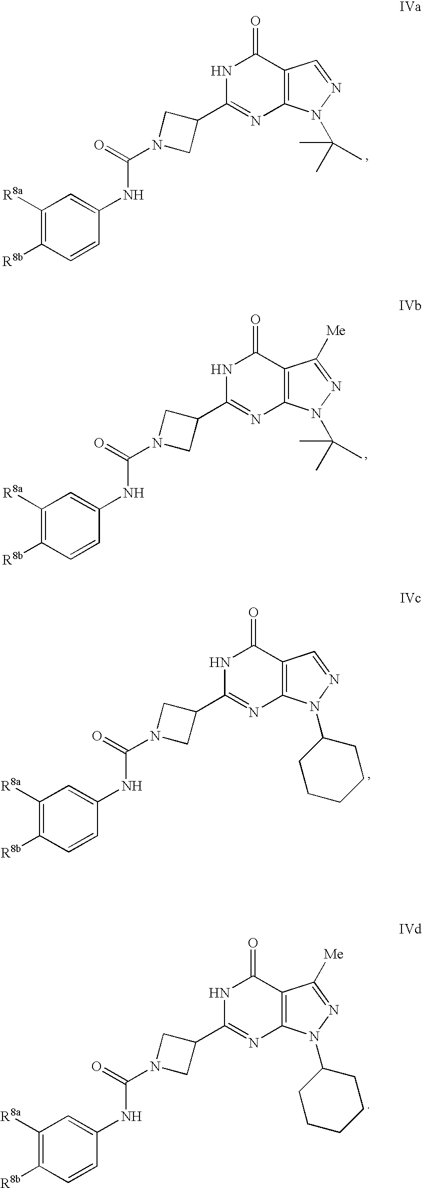

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl. In another embodiment with respect to the compounds of Formulae IVa-IVd, each of R^{8a} and R^{8b} may be selected from H, C₁-C₆ alkyl, halo, CN, C₁-C₆ alkoxy, C₁-C₆ haloalkyl, C₁-C₆ haloalkoxy, amino, dialkylamino, heterocycloalkyl and heterocycloalkylalkyl.

In another embodiment, with respect to compounds of formula Ic, the compound is according to formulae Va, Vb, Vc or Vd:

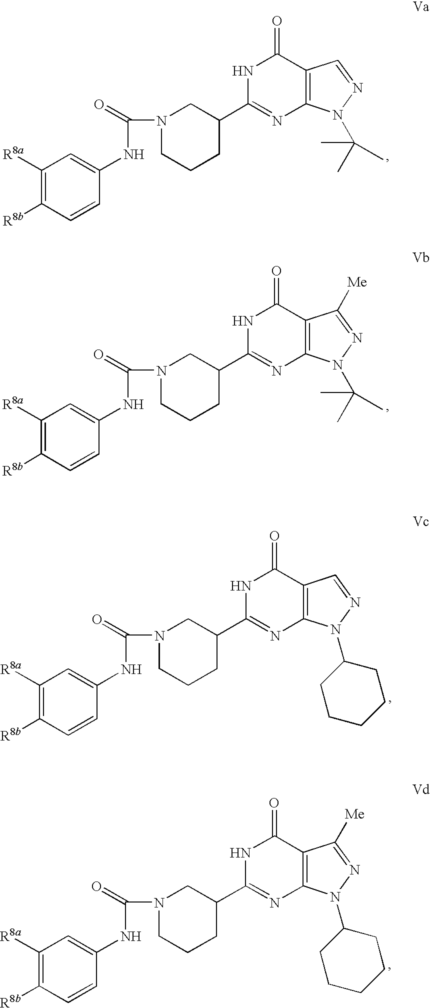

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In one embodiment, with respect to compounds of formulae IVa-Vd, R^{8a} is H, Me, NMe₂, Cl or F; and R^{8b} is H, Me, Cl or F.

In another embodiment, with respect to compounds of formulae IVa-Vd, R^{8a} is H; and R^{8b} is H, Me, Cl, F, NMe₂, OMe, i-Pr, t-Bu, OCF₃, CF₃, CN, morpholin-1-yl, piperazin-1-yl, N-methylpiperazin-1-yl or N-isopropylpiperazin-1-yl.

In another embodiment, with respect to compounds of formula Ic, the compound is according to formulae VIa, VIb, VIc or VId:

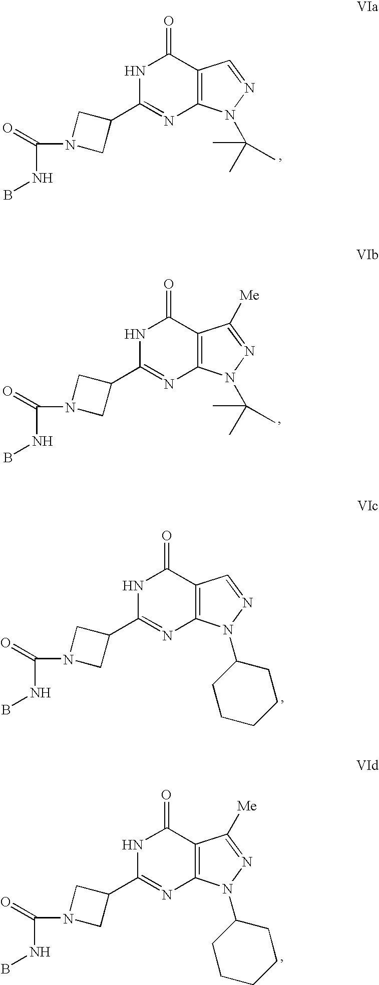

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formula Ic, the compound is according to formulae VIIa, VIIb, VIIc or VIId:

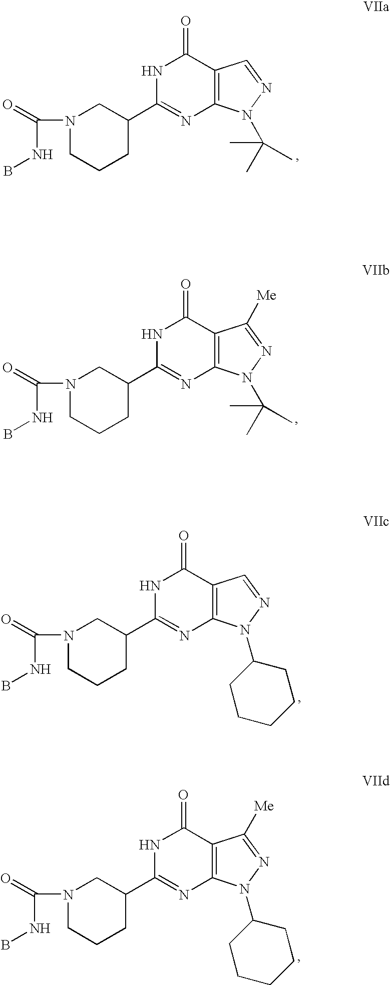

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formulae VIa-VIId, B is selected from t-Bu, i-Pr, n-Bu, cyclohexyl, cyclopentyl, cyclohexylmethyl, cyclopentylmethyl, piperidinyl, and benzyl.

In another embodiment, with respect to compounds of formulae VIa-VIId, B is selected from

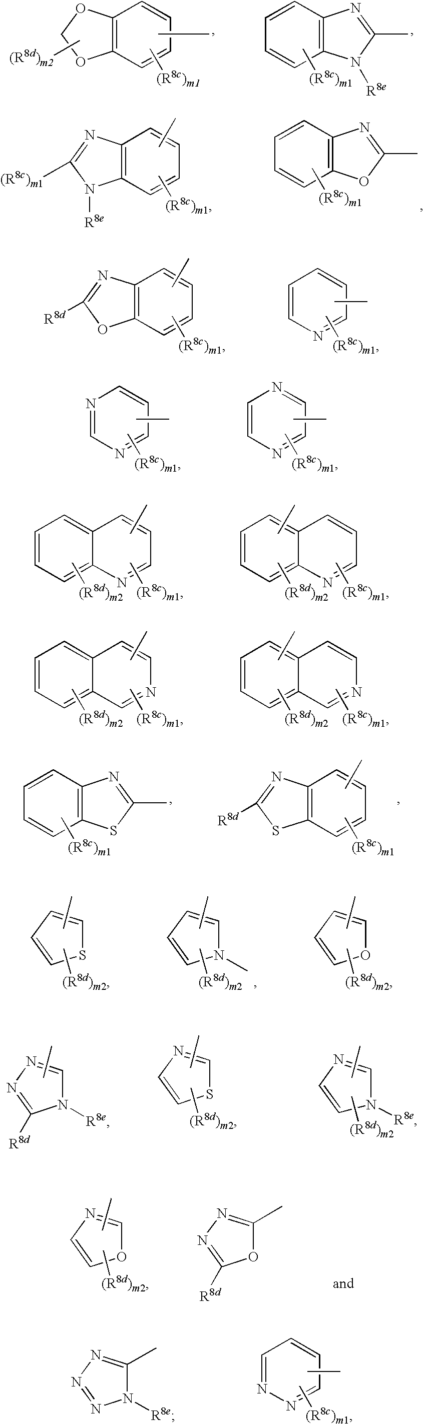

wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴, and SO₂R⁴, or
each one of R^{8c} or R^{8d} is independently selected from C₁-C₆ alkyl, halo C₁-C₆ alkyl, C₁-C₆ alkoxy, and halo C₁-C₆ alkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl;
and each of subscript m1 and m2 is independently selected from 0, 1 and 2.

In one embodiment, with respect to compounds of formulae VIa-VIId, B is as described above and each of R^{8c} or R^{8d} is independently selected from H, Me, t-Bu, F, C₁ or CF₃; and each of subscript m1 and m2 is independently selected from 1 and 2.

In another embodiment, with respect to compounds of formulae VIa-VIId, B is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae VIa-VIId, B is as described above and each of R^{8c} or R^{8d} is independently selected from H, Ph, morpholinylmethyl, piperidinylmethyl, piperazinylmethyl, N-Me-piperazinylmethyl, and N-i-Pr-piperazinylmethyl; and each of subscript m1 and m2 is independently selected from 1 and 2.

In another embodiment, with respect to compounds of formulae VIa-VIId, B is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In one embodiment, the compound is according to formula

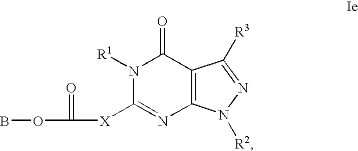

and wherein B, X, R¹, R², and R³ are as described for formulae Ia-Ih.

In one embodiment, with respect to compounds of formula Ie, the compound is according to formulae VIIIa, VIIIh, VIIIc or VIIId:

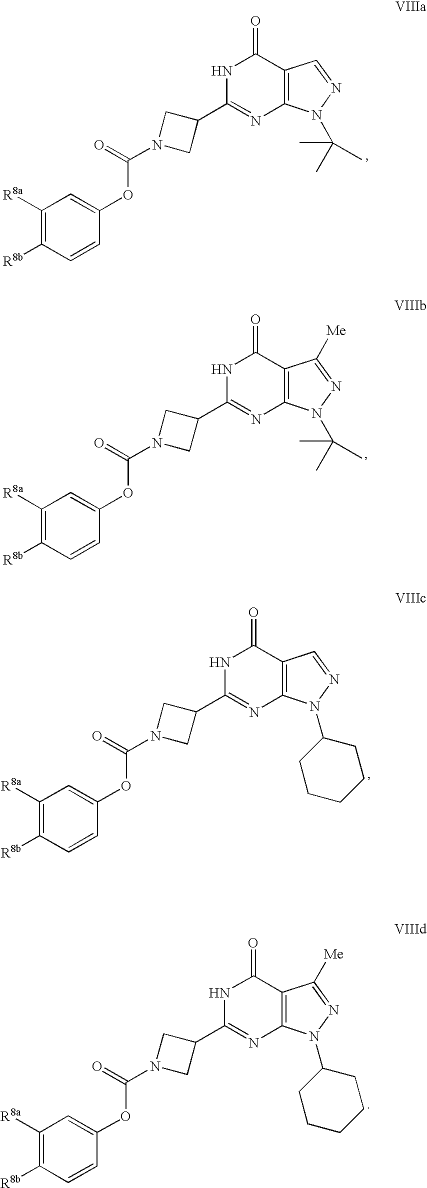

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In another embodiment, with respect to compounds of formula Ie, the compound is according to formulae IXa, IXb, IXc or IXd:

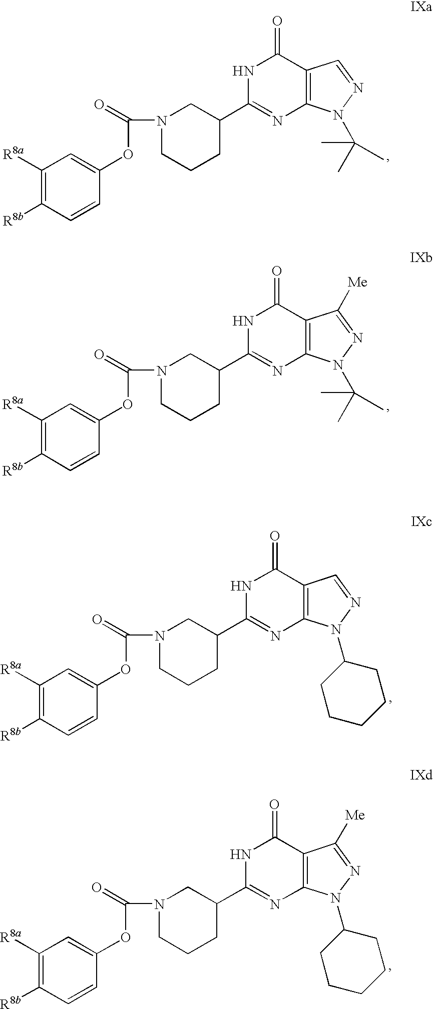

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In one embodiment, with respect to compounds of formulae VIIIa-IXd, R^{8a} is H, Me, NMe₂, Cl or F; and R^{8b} is H, Me, Cl or F.

In another embodiment, with respect to compounds of formulae VIIIa-IXd, R^{8a} is H; and R^{8b} is H, Me, Cl, F, NMe₂, OMe, i-Pr, t-Bu, OCF₃, CF₃, CN, morpholin-1-yl, piperazin-1-yl, N-methylpiperazin-1-yl or N-isopropylpiperazin-1-yl. In a particular embodiment, R^{8a} is H and R^{8b} is H, F, NMe₂, or i-Pr.

In another embodiment, with respect to compounds of formula Ie, the compound is according to formulae Xa, Xb, Xc or Xd:

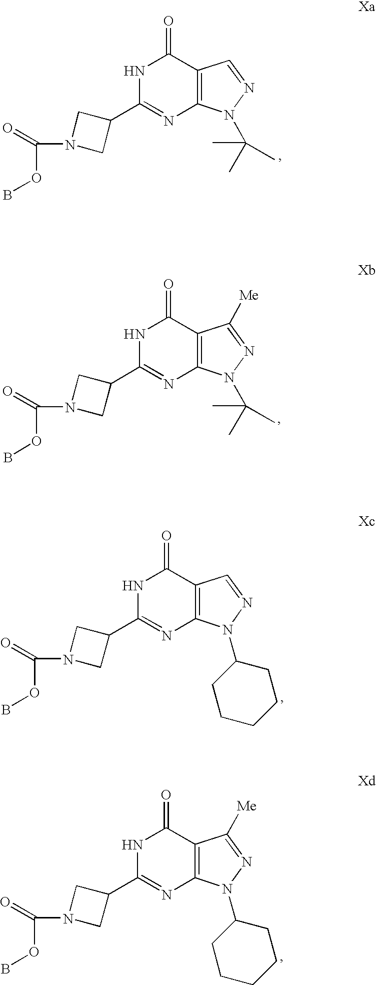

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formula Ie, the compound is according to formulae XIa, XIb, XIc or XId:

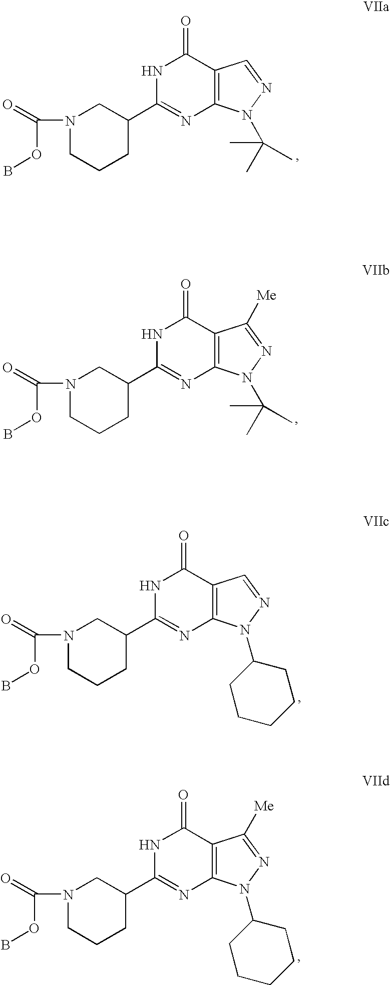

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formulae Xa-XId, B is selected from t-Bu, i-Pr, n-Bu, cyclohexyl, cyclopentyl, cyclohexylmethyl, cyclopentylmethyl, piperidinyl and benzyl.

In another embodiment, with respect to compounds of formulae Xa-XId, B is selected from

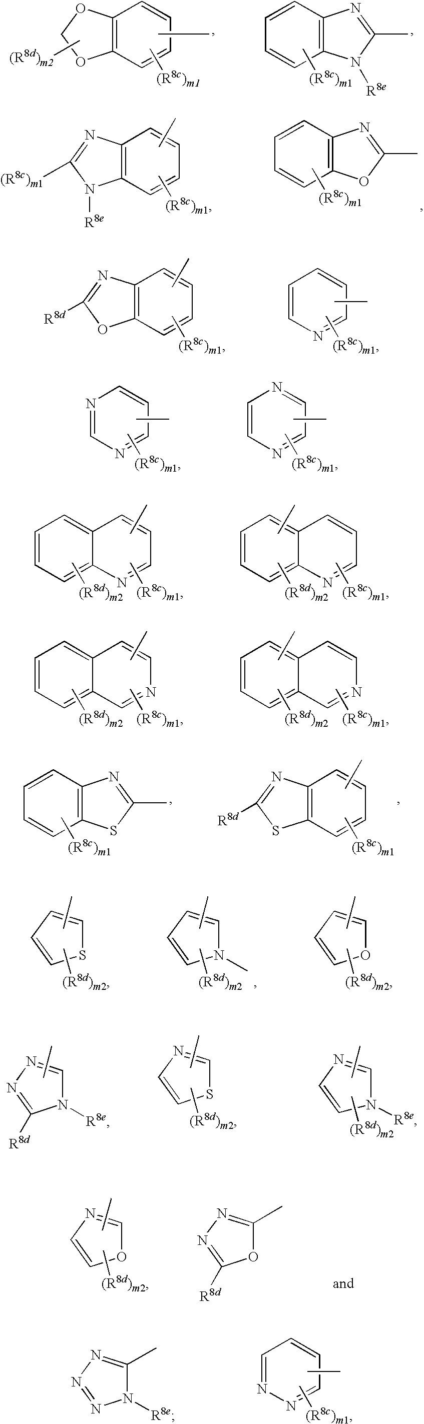

wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴, and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl;
and each of subscript m1 and m2 is independently selected from 0, 1 and 2.

In one embodiment, with respect to compounds of formulae Xa-XId, B is as described above and each of R^{8c} or R^{8d} is independently selected from H, Me, t-Bu, F, Cl, and CF₃; and each of subscript m1 and m2 is independently selected from 1 and 2.

In another embodiment, with respect to compounds of formulae Xa-XId, B is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae Xa-XId, B is as described above and each of R^{8c} or R^{8d} is independently selected from H, Ph, morpholinylmethyl, piperidinylmethyl, piperazinylmethyl, N-Me-piperazinylmethyl, N-i-Pr-piperazinylmethyl; and each of subscript m1 and m2 is independently selected from 1 and 2.

In another embodiment, with respect to compounds of formulae Xa-XId, B is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In one embodiment, the compound is according to formula

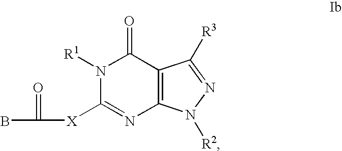

and wherein B, X, R¹, R², and R³ are as described for formulae Ia-Ih.

In one embodiment, with respect to compounds of formula Ib, the compound is according to formulae XIIa, XIlb, XIIc or XIId:

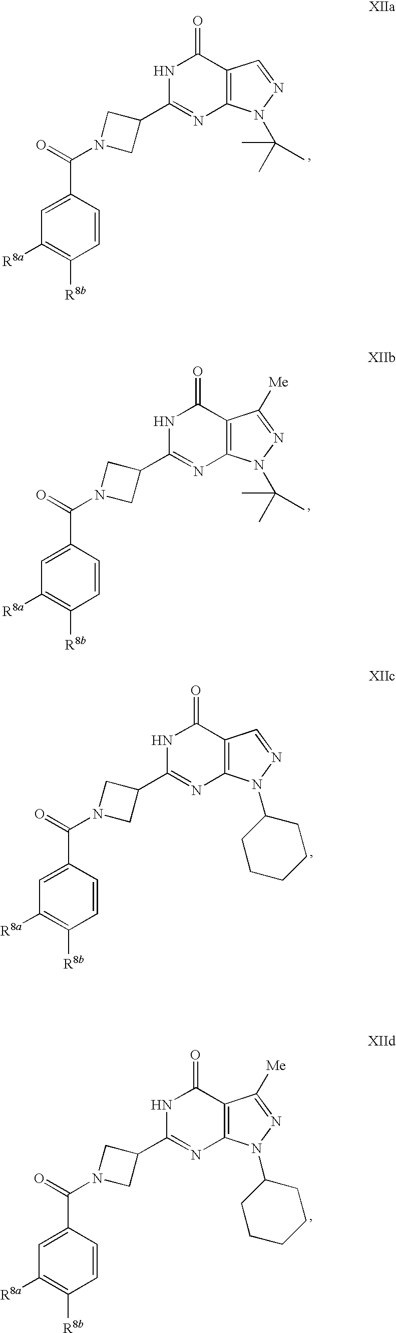

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In another embodiment, with respect to compounds of formula Ib, the compound is according to formulae XIIIa, XIIIb, XIIIc or XIIId:

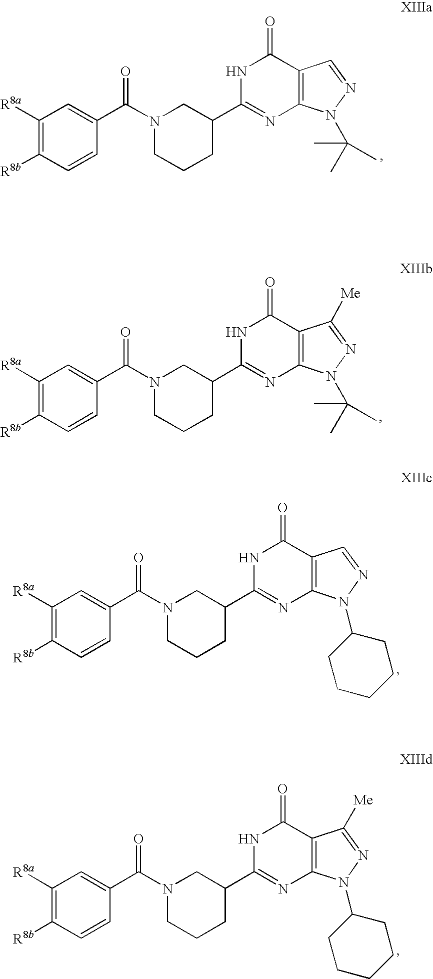

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In one embodiment, with respect to compounds of formulae XIIIa-XIIId, R^{8a} is H, Me, NMe2, Cl or F; and R^{8b} is H, Me, Cl or F.

In another embodiment, with respect to compounds of formulae XIIa-XIIId, R^{8a} is H; and

R^{8b} is H, Me, Cl, F, NMe2, OMe, i-Pr, t-Bu, OCF3, CF3, CN, morpholin-1-yl, piperazin-1-yl, N-methylpiperazin-1-yl or N-isopropylpiperazin-1-yl.

In another embodiment, with respect to compounds of formula Ib, the compound is according to formulae XIVa, XIVb, XIVc or XIVd:

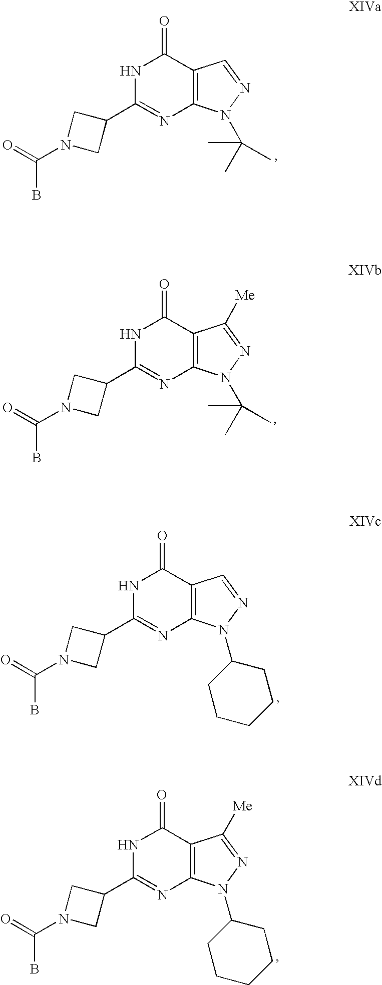

wherein B is selected from alkyl, cycloalkyl, cycloalkylalkyl, heterocycloalkyl, heterocycloalkylalkyl, heteroaryl and aralkyl.

In another embodiment, with respect to compounds of formula Ib, the compound is according to formulae XVa, XVb, XVc or XVd:

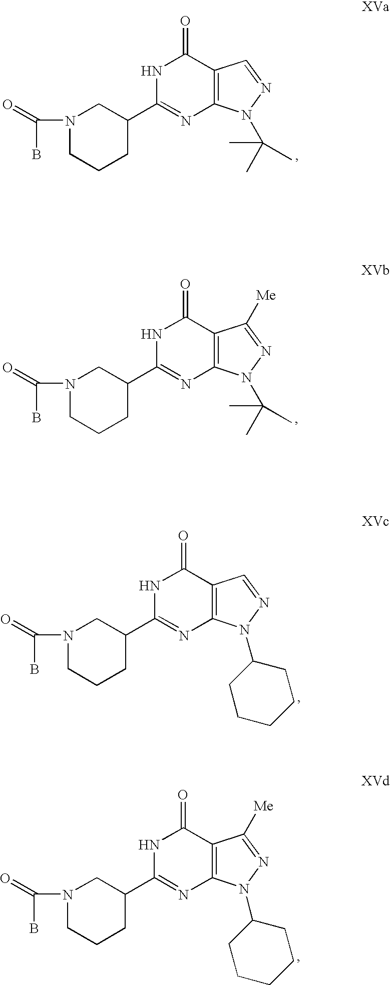

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formulae XIVa-XVd, B is selected from t-Bu, i-Pr, n-Bu, cyclohexyl, cyclopentyl, cyclohexylmethyl, cyclopentylmethyl, piperidinyl and benzyl.

In another embodiment, with respect to compounds of formulae XIVa-XVd, B is selected from

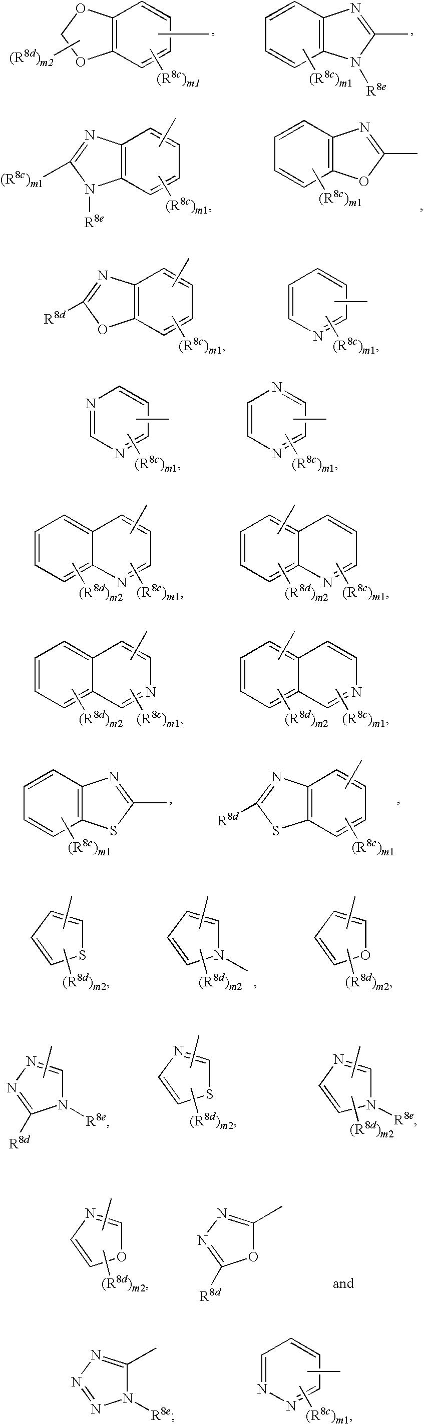

wherein each one of R^{8c}, or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴ and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e}, is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl;
and each of subscript m1 and m2 is independently selected from 0, 1 and 2.

In one embodiment, with respect to compounds of formulae XIVa-XVd, B is as described above and each of R^{8c} or R^{8d} is independently selected from H, Me, t-Bu, F, Cl, and CF₃; and each of subscript m1 and m2 is independently selected from 1 and 2.

In another embodiment, with respect to compounds of formulae XIVa-XVd, B is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae XIVa-XVd, B is as described above and each of R^{8c} or R^{8d} is independently selected from H, Ph, morpholinylmethyl, piperidinylmethyl, piperazinylmethyl, N-Me-piperazinylmethyl, N-i-Pr-piperazinylmethyl; and each of subscript m1 and m2 is independently selected from 1 and 2.

In another embodiment, with respect to compounds of formulae XIVa-XVd, B is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In one embodiment, the compound is according to formula

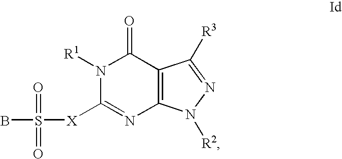

and wherein B, X, R¹, R², and R³ are as described for formulae Ia-Ih.

In one embodiment, with respect to compounds of formula Id, the compound is according to formulae XVIa, XVIb, XVIc or XVId:

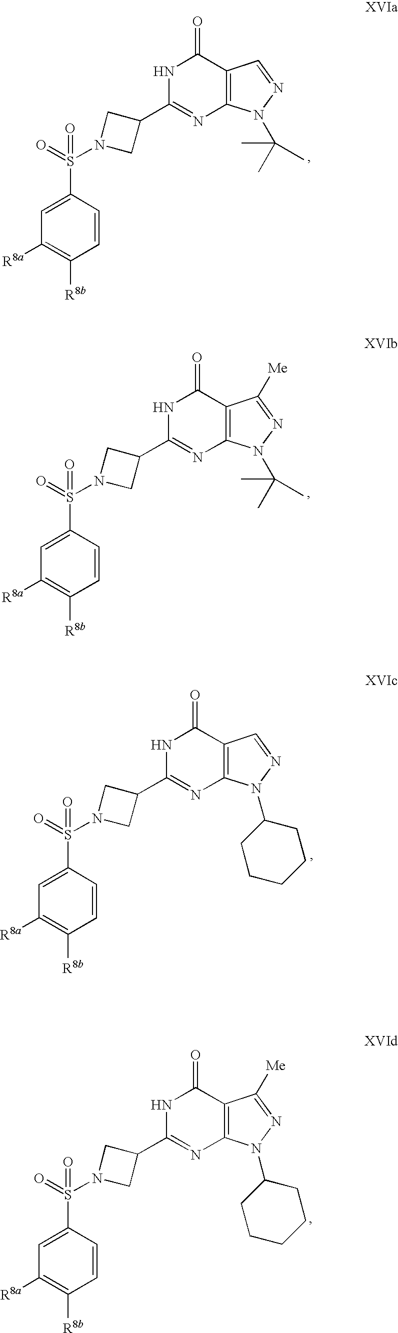

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In another embodiment, with respect to compounds of formula Id, the compound is according to formulae XVIIa, XVIIb, XVIIc or XVIId:

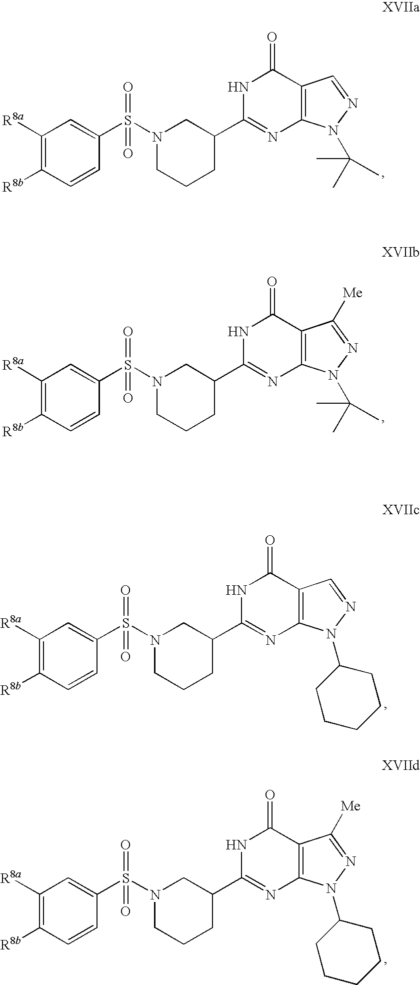

wherein R^{8a} and R^{8b} are independently selected from H, alkyl, halo, CN, alkoxy, haloalkyl, haloalkoxy, amino, dialkylamino, heterocycloalkyl, and heterocycloalkylalkyl.

In one embodiment, with respect to compounds of formulae XVIa-XVIId, R^{8a} is H, Me, NMe₂, Cl or F; and R^{8b} is H, Me, Cl or F.

In another embodiment, with respect to compounds of formulae XVIa-XVIId, R^{8a} is H; and R^{8b} is H, Me, Cl, F, NMe₂, OMe, i-Pr, t-Bu, OCF₃, CF₃, CN, morpholin-1-yl, piperazin-1-yl, N-methylpiperazin-1-yl or N-isopropylpiperazin-1-yl.

In another embodiment, with respect to compounds of formula Id, the compound is according to formulae XVIIIa, XVIIIb, XVIIIc or XVIIId:

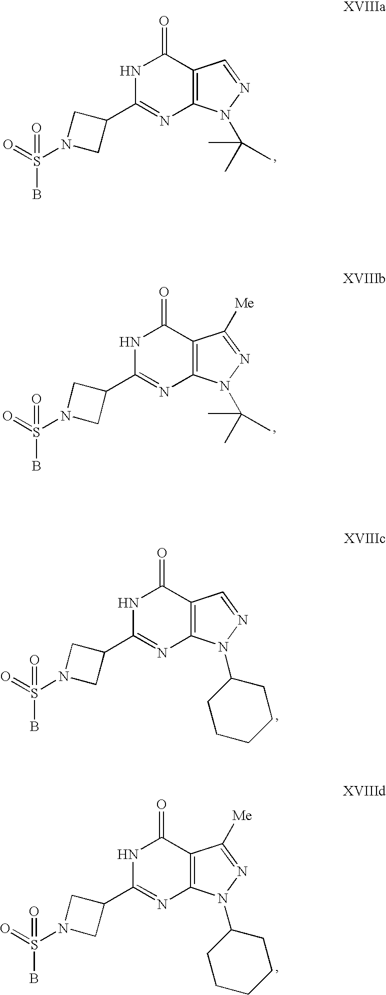

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formula Id, the compound is according to formulae XIXa, XIXb, XIXc or XIXd:

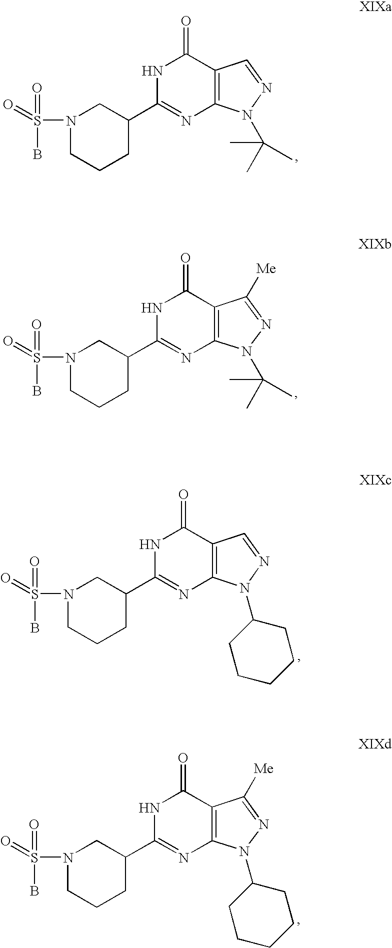

wherein B is selected from C₁-C₆ alkyl, cycloalkyl, cycloalkylalkyl, heterocycloalkyl, heterocycloalkylalkyl, heteroaryl and aralkyl.

In another embodiment, with respect to compounds of formulae XVIIIa-XIXd, B is selected from t-Bu, i-Pr, n-Bu, cyclohexyl, cyclopentyl, cyclohexylmethyl, cyclopentylmethyl, piperidinyl, and benzyl.

In another embodiment, with respect to compounds of formulae XVIIIa-XIXd, B is selected from

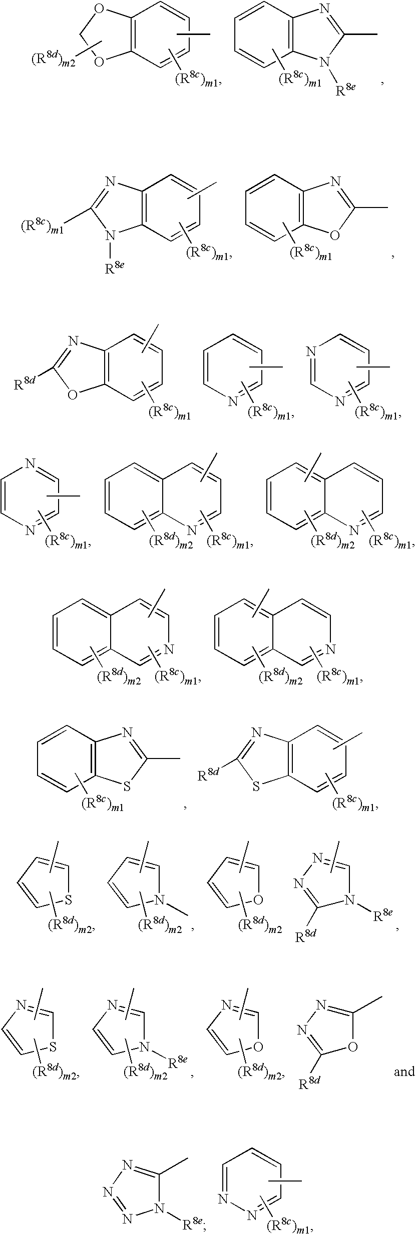

wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴ and SO₂R⁴, or
each one of R^{8c} or R^{5d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl;
and each of subscript m1 and m2 is independently selected from 0, 1 and 2.

In one embodiment, with respect to compounds of formulae XVIIIa-XIXd, B is as described above and each of R^{8c} or R^{8d} is independently selected from H, Me, t-Bu, F, Cl, and CF₃; and each of subscript m1 and m2 is independently selected from 1 and 2.

In another embodiment, with respect to compounds of formulae XVIIIa-XIXd, B is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae XVIIIa-XIXd, B is as described above and each of R^{8c} or R^{8d} is independently selected from H, Ph, morpholinylmethyl, piperidinylmethyl, piperazinylmethyl, N-Me-piperazinylmethyl, N-i-Pr-piperazinylmethyl; and each of subscript m1 and m2 is independently selected from 1 and 2.

In another embodiment, with respect to compounds of formulae XVIIIa-XIXd, B is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In one embodiment, the compound is according to formula

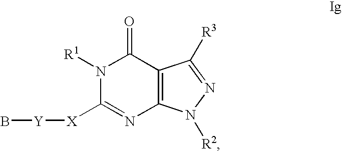

and wherein B, X, Y, R¹, R², and R³ are as described for formulae Ia-Ih.

In one embodiment, with respect to compounds of formula Ig, Y is a bond.

In one embodiment, with respect to compounds of formula Ig, the compound is according to formulae XXa, XXb, XXc or XXd:

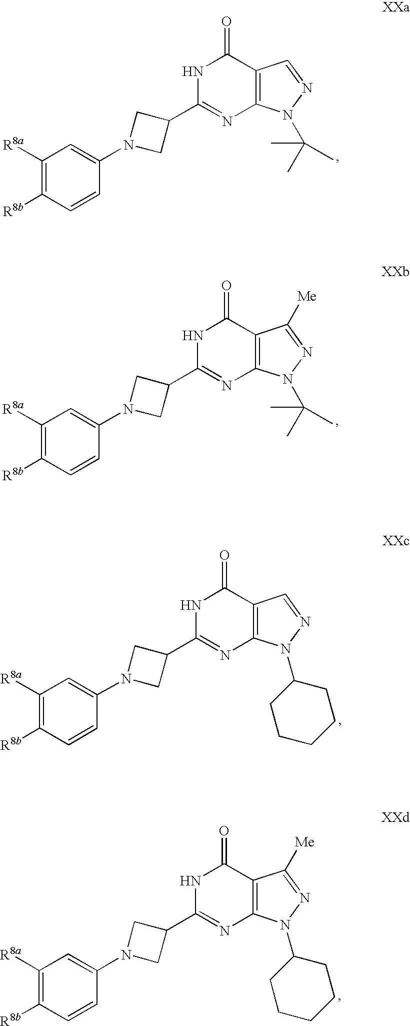

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In another embodiment, with respect to compounds of formula Ig, the compound is according to formulae XXIa, XXIb, XXIc or XXId:

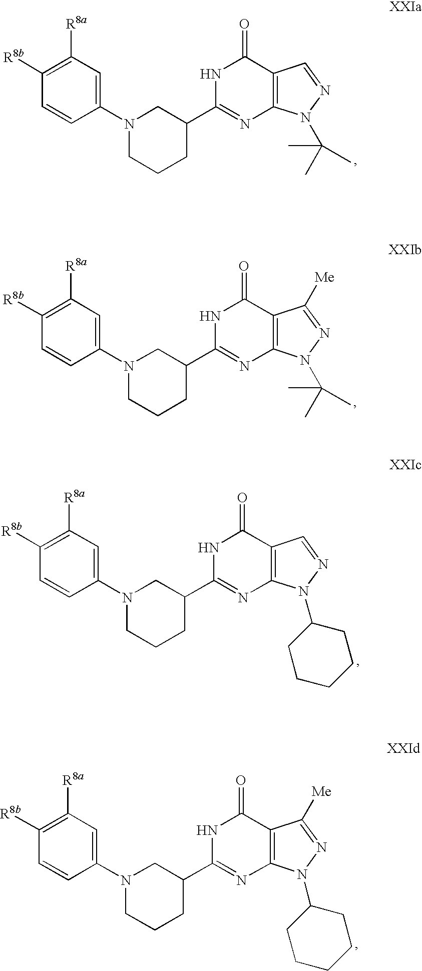

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

In one embodiment, with respect to compounds of formulae XXa-XXId, R^{8a} is H, Me, NMe₂, Cl or F; and R^{8b} is H, Me, Cl or F.

In another embodiment, with respect to compounds of formulae XXa-XXId, R^{8a} is H; and R^{8b} is H, Me, Cl, F, NMe₂, OMe, i-Pr, t-Bu, OCF₃, CF₃, CN, morpholin-1-yl, piperazin-1-yl, N-methylpiperazin-1-yl or N-isopropylpiperazin-1-yl.

In another embodiment, with respect to compounds of formula Ig, the compound is according to formulae XXIIa, XXIIb, XXIIc or XXIId:

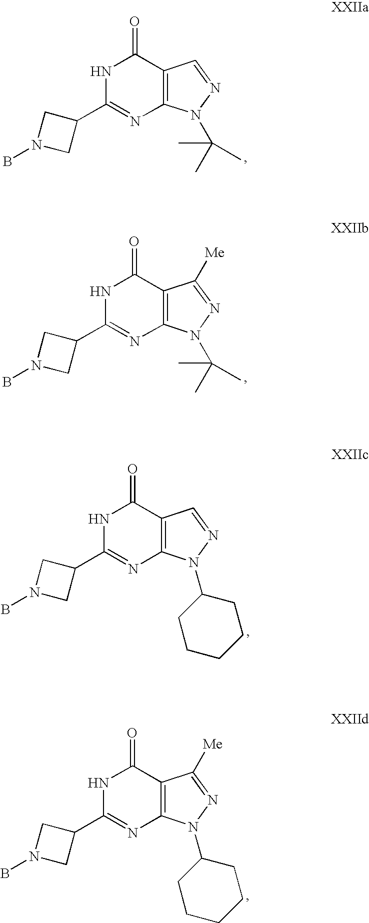

wherein B is selected from C₁-C₆ alkyl, cycloalkyl, cycloalkylalkyl, heterocycloalkyl, heterocycloalkylalkyl, aryl, heteroaryl, heteroaryl and aralkyl.

In another embodiment, with respect to compounds of formula Ig, the compound is according to formulae XXIIIa, XXIIIb, XXIIIc or XXIIId:

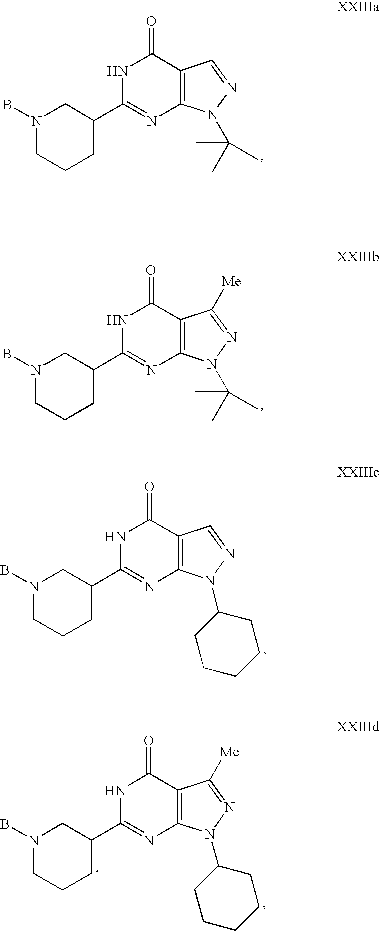

wherein B is selected from C₁-C₆ alkyl, cycloalkyl, cycloalkylalkyl, heterocycloalkyl, heterocycloalkylalkyl, aryl, heteroaryl, heteroaryl and aralkyl.

In another embodiment, with respect to compounds of formulae XXIIa-XXIIId, B is selected from t-Bu, i-Pr, n-Bu, cyclohexyl, cyclopentyl, cyclohexylmethyl, cyclopentylmethyl, piperidinyl, and benzyl.

In one embodiment, with respect to compounds of formula Ig, Y is substituted or unsubstituted aryl.

In one embodiment, with respect to compounds of formula Ig, Y is substituted or unsubstituted heteroaryl.

In one embodiment, with respect to compounds of formula Ig, the compound is according to formulae XXIVa, XXIVb, XXIVc or XXIVd:

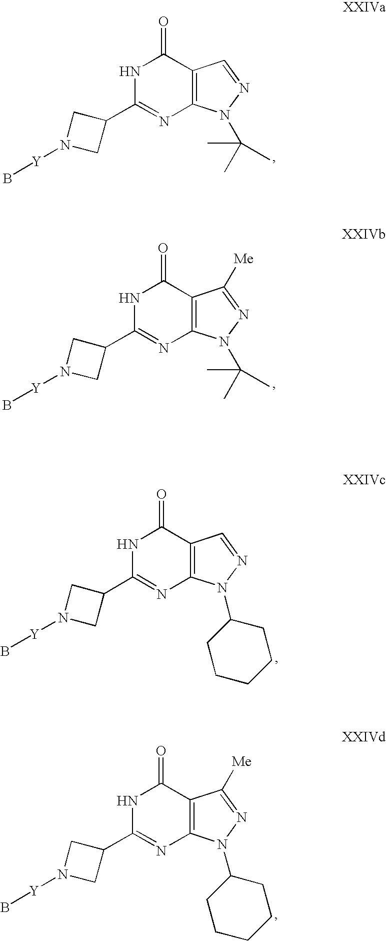

wherein Y is substituted or unsubstituted heteroaryl; and B is selected from H, C₁-C₆ alkyl, C₁-C₆ haloalkyl, halo, CN, NO₂, aryl, heteroaryl, cycloalkyl, cycloalkylalkyl, heterocycloalkyl, heterocycloalkylalkyl, heterocycloalkylphenyl and aralkyl.

In another embodiment, with respect to compounds of formula Ig, the compound is according to formulae XXVa, XXVb, XXVc or XXVd:

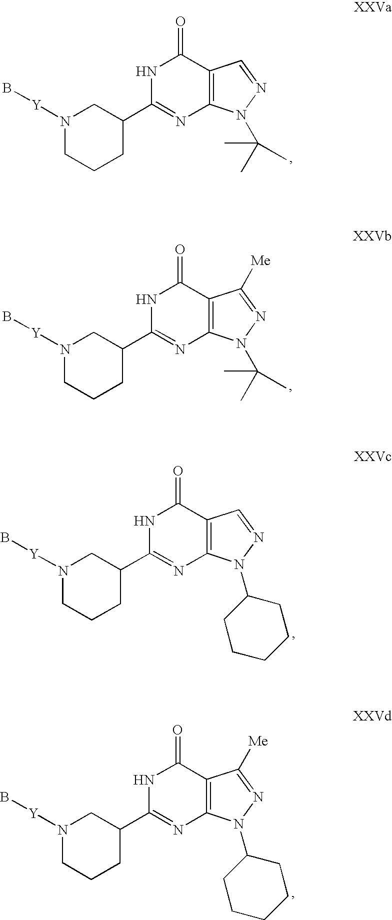

wherein Y is substituted or unsubstituted heteroaryl; and B is selected from H, C₁-C₆ alkyl, C₁-C₆ haloalkyl, halo, CN, NO₂, aryl, heteroaryl, cycloalkyl, cycloalkylalkyl, heterocycloalkyl, heterocycloalkylalkyl, heterocycloalkylphenyl, and aralkyl.

In one embodiment, with respect to compounds of formulae XXIVa-XXVd, Y is selected from pyridyl, pyrimidyl, pyrazinyl, quinolinyl, isoquinolinyl, benzimidazolyl, benzoxazolyl, benzthiazolyl, benz[1,3]dioxalyl, thiophenyl, pyrrolidinyl, furanyl, triazolyl, thiazolyl, imidazolyl, oxazolyl, oxadiazolyl, and tetrazolyl.

In one embodiment, with respect to compounds of formulae XXIVa-XXVd, B is selected from H, Me, t-Bu, F, Cl, CF₃, NO₂, Ph, morpholin-1-yl, piperidin-1-yl, piperazin-1-yl, N-Me-piperazin-1-yl, N-i-Pr-piperazin-1-yl, morpholinylmethyl, piperidinylmethyl, piperazinylmethyl, N-Me-piperazinylmethyl, N-i-Pr-piperazinylmethyl, morpholinylphenyl, piperidinylphenyl, piperazinylphenyl, N-Me-piperazinylphenyl, and N-i-Pr-piperazinylphenyl.

In one embodiment, with respect to compounds of formulae XXIVa-XXVd, the group B—Y— is selected from

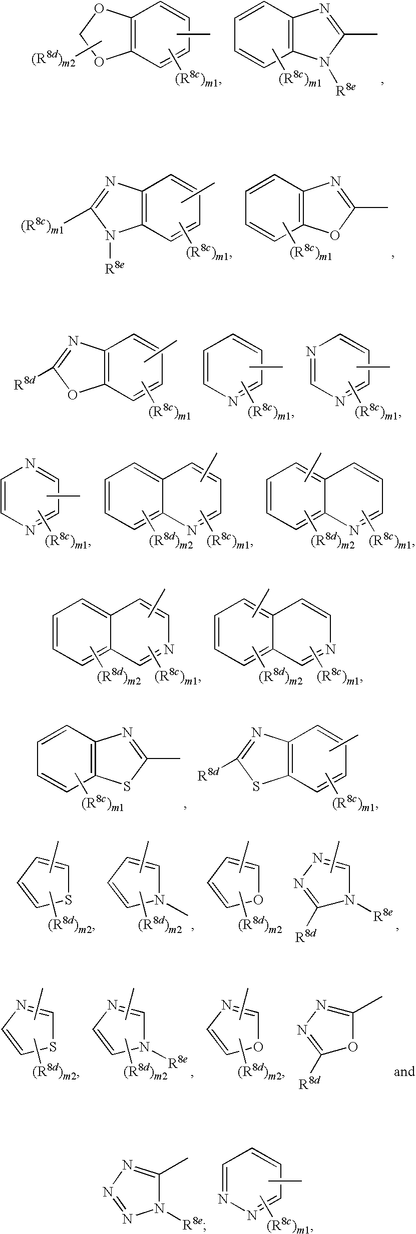

wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴, and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl;
and each of subscript m1 and m2 is independently selected from 0, 1 and 2.

In one embodiment, with respect to compounds of formulae XXIVa-XXVd, the group B—Y— is as described above and each of R^{8c} or R^{8d} is independently selected from H, Me, t-Bu, F, Cl, CF₃; and each of subscript m1 and m2 is independently selected from 1 and 2.

In another embodiment, with respect to compounds of formulae XXIVa-XXVd, the group B—Y— is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae XXIVa-XXVd, the group B—Y— is as described above and each of R^{8c} or R^{8d} is independently selected from H, Ph, morpholinylmethyl, piperidinylmethyl, piperazinylmethyl, N-Me-piperazinylmethyl, N-i-Pr-piperazinylmethyl; and each of subscript m1 and m2 is independently selected from 1 and 2.

In another embodiment, with respect to compounds of formulae XXIVa-XXVd, the group B—Y— is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In another embodiment, with respect to compounds of formulae XXIVa-XXVd, the group B—Y— is selected from

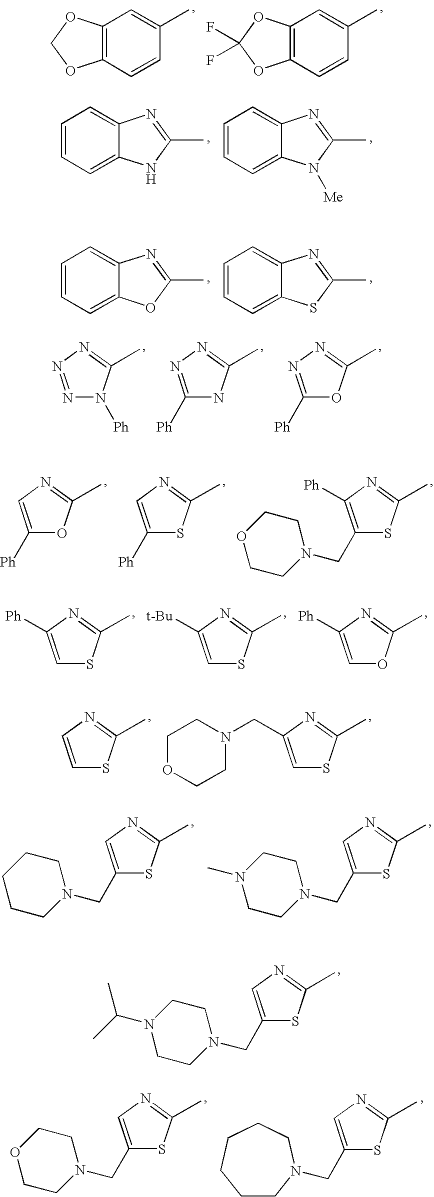

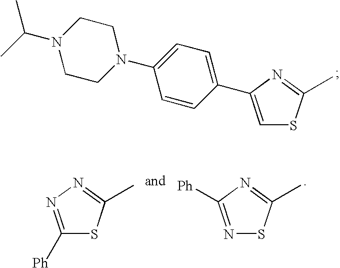

In one embodiment, with respect to compounds of formulae XXIIa-XXIIId, B is selected from t-Bu, i-Pr, n-Bu, cyclohexyl, cyclopentyl, cyclohexylmethyl, cyclopentylmethyl, piperidinyl, benzo[1,3]dioxalyl, benzimidazol-2-yl, methylbenzimidazol-2-yl, trifluoromethylbenzimidazol-2-yl, and benzyl. In a particular embodiment B is benzimidazol-2-yl, methylbenzimidazol-2-yl, or trifluoromethylbenzimidazol-2-yl. In a more particular embodiment B is benzimidazol-2-yl, or trifluoromethylbenzimidazol-2-yl.

In another embodiment, with respect to compounds of formulae XXIIa-XXIIId, B is benzthiazol-2-yl or benzoxazol-2-yl.

In yet another embodiment, with respect to compounds of formulae XXIIa-XXIIId, B is oxazol-2-yl, oxadiazolyl, thiazol-2-yl, imidazol-2-yl, or tetrazolyl.

In yet another embodiment, with respect to compounds of formulae XXIIa-XXIIId, B is substituted oxazol-2-yl, substituted oxadiazolyl, substituted thiazol-2-yl, substituted imidazol-2-yl, or substituted tetrazolyl and the substitution is selected from methyl, t-Bu, phenyl, morpholinomethyl, piperidinomethyl, or alkylpiperazinomethyl.

In yet another embodiment, with respect to compounds of formulae XXIIa-XXIId, B is pyridyl.

In one embodiment, the compound is according to formula

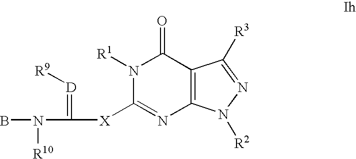

and wherein B, X, D, R¹, R², R³, R⁹, and R¹⁰ are as described for formulae Ia-1 h.

In one embodiment, with respect to compounds of formula Ih, R¹⁰ is H.

In one embodiment, with respect to compounds of formula Ih, the compound is according to formulae XXVIa, XXVIb, XXVIc or XXVId:

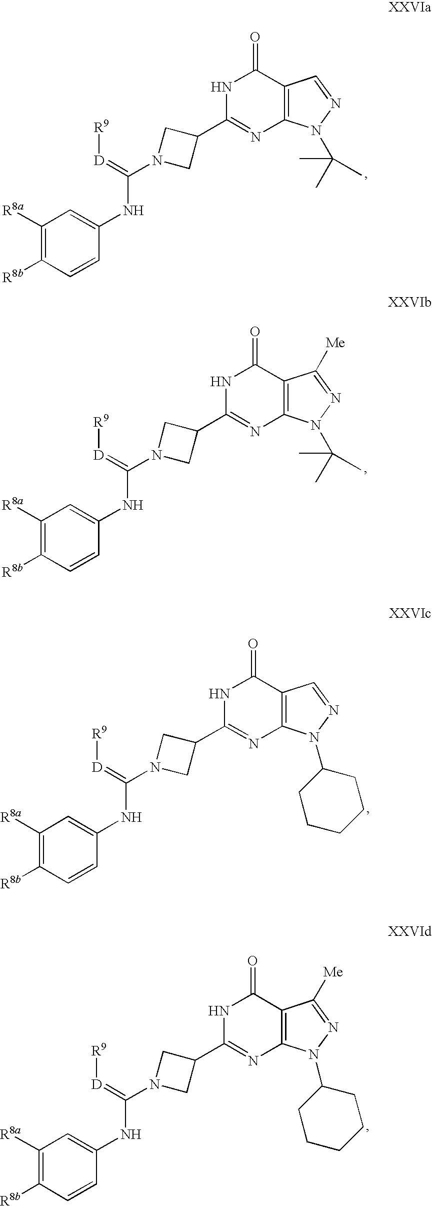

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl; and the group D-R⁹ is N—CN or CH—NO₂.

In another embodiment, with respect to compounds of formula Ih, the compound is according to formulae XXVIIa, XXVIIb, XXVIIc or XXVIId:

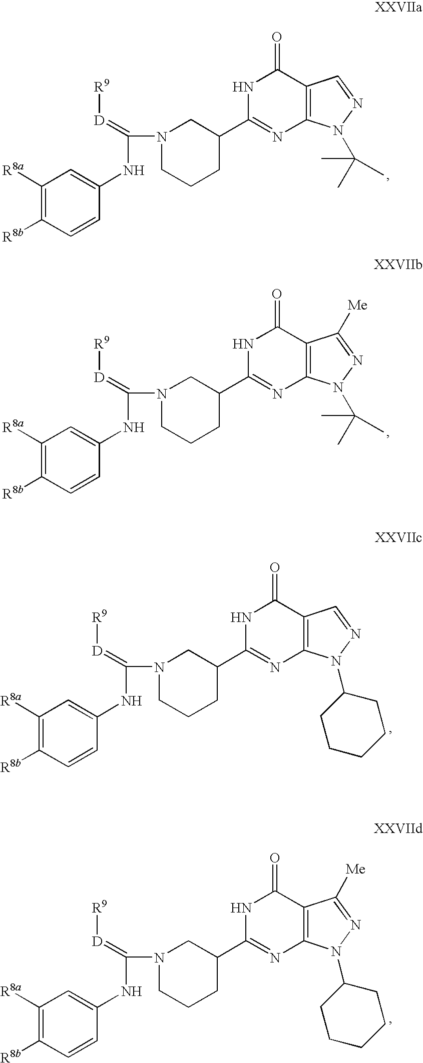

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl; and the group D-R⁹ is N—CN or CH—NO₂.

In one embodiment, with respect to compounds of formulae XXVIa-XXVIId, R^{8a} is H, Me, NMe₂, Cl or F; and R^{8b} is H, Me, Cl or F.

In another embodiment, with respect to compounds of formulae XXVIa-XXVIId, R^{8a} is H; and R^{9b} is H, Me, Cl, F, NMe₂, OMe, i-Pr, t-Bu, OCF₃, CF₃, CN, morpholin-1-yl, piperazin-1-yl, N-methylpiperazin-1-yl or N-isopropylpiperazin-1-yl.

In another embodiment, with respect to compounds of formula Ih, the compound is according to formulae XXVIIIa, XXVIIIb, XXVIIIc or XXVIIId:

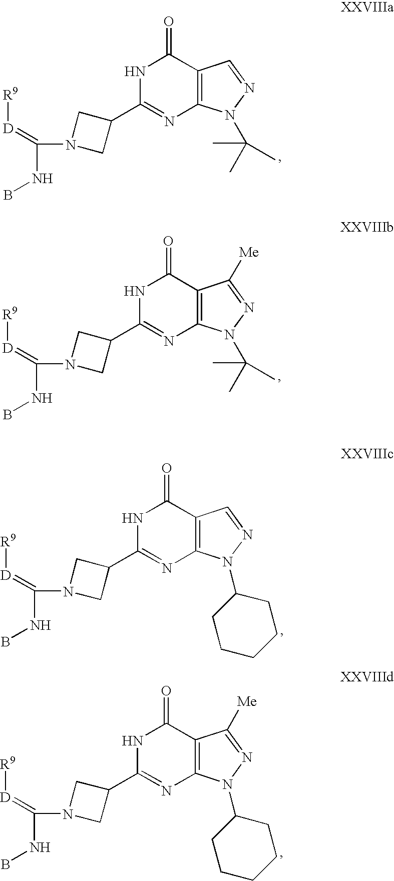

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl; and the group D-R⁹ is N—CN or CH—NO₂.

In another embodiment, with respect to compounds of formula Ih, the compound is according to formulae XXIXa, XXIXb, XXIXc or XXIXd:

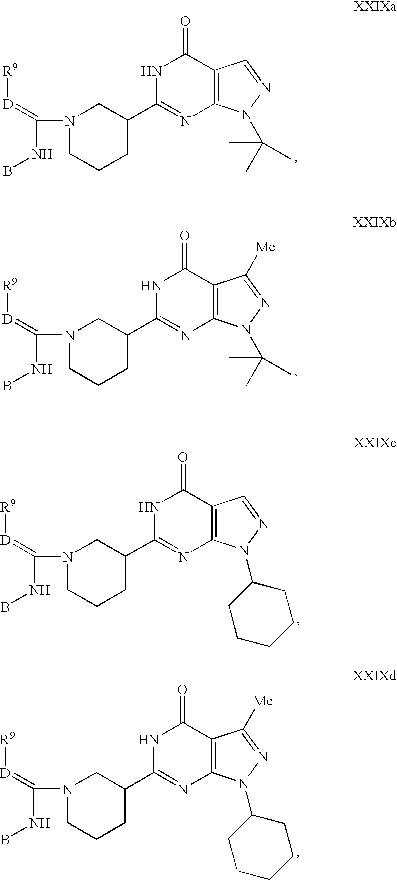

wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl; and the group D-R⁹ is N—CN or CH—NO₂.

In another embodiment, with respect to compounds of formulae XXVIIIa-XXIXd, B is selected from t-Bu, i-Pr, n-Bu, cyclohexyl, cyclopentyl, cyclohexylmethyl, cyclopentylmethyl, piperidinyl, and benzyl.

In another embodiment, with respect to compounds of formulae XXVIIIa-XXIXd, B is selected from

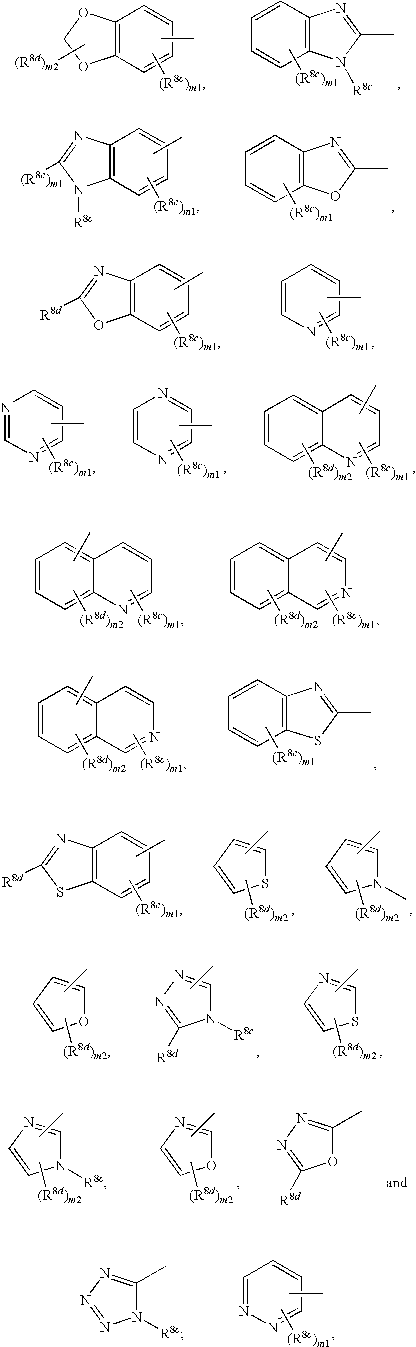

wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴ and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl;
and each of subscript m1 and m2 is independently selected from 0, 1 and 2.

In one embodiment, with respect to compounds of formulae XXVIIIa-XXIXd, B is as described above and each of R^{8c} or R^{8d} is independently selected from H, Me, t-Bu, F, Cl, and CF₃; and each of subscript m1 and m2 is independently selected from 1 and 2.

In another embodiment, with respect to compounds of formulae XXVIIIa-XXIXd, B is as described above and each of R^{8c} or R^{8d} is independently selected from H, Ph, morpholin-1-yl, piperidin-1-yl, piperazin-1-yl, N-Me-piperazin-1-yl, N-i-Pr-piperazin-1-yl; and each of subscript m1 and m2 is independently selected from 1 and 2.

In another embodiment, with respect to compounds of formulae XXVIIIa-XXIXd, B is as described above and each of R^{8c} or R^{9d} is independently selected from H, Ph, morpholinylmethyl, piperidinylmethyl, piperazinylmethyl, N-Me-piperazinylmethyl, N-i-Pr-piperazinylmethyl; and each of subscript m1 and m2 is independently selected from 1 and 2.

In another embodiment, with respect to compounds of formulae XXVIIIa-XXIXd, B is as described above and each of R^{8c} or R^{8d} is independently selected from H, Ph, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

In another embodiment, with respect to compounds of formulae Ia-Ih, the compound is selected from:
N-(benzo[d][1,3]dioxol-5-yl)-3-(1-tert-butyl-3-methyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(4-isopropylpiperazin-1-yl)phenyl)azetidine-1-carboxamide;
3-(1-cyclohexyl-4-oxo-4,5-dihydro-1H-pyrazolo[3,4-d]pyrimidin-6-yl)-N-(4-(4-methylpiperazin-1-yl)phenyl)azetidine-1-carboxamide;
3-(1-cyclohexyl-4-oxo-4,5-dihydro-1H-pyrazolo[3,4-d]pyrimidin-6-yl)-N-(3,4-dimethylphenyl)azetidine-1-carboxamide;
3-(1-cyclohexyl-4-oxo-4,5-dihydro-1H-pyrazolo[3,4-d]pyrimidin-6-yl)-N-(cyclohexylmethyl)azetidine-1-carboxamide;
3-(1-tert-butyl-3-methyl-4-oxo-4,5-dihydro-1H-pyrazolo[3,4-d]pyrimidin-6-yl)-N-phenylazetidine-1-carboxamide;
3-(1-cyclohexyl-4-oxo-4,5-dihydro-1H-pyrazolo[3,4-d]pyrimidin-6-yl)-N-(3-(dimethylamino)phenyl)azetidine-1-carboxamide;
N-(3-chlorophenyl)-3-(1-cyclohexyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(dimethylamino)phenyl)azetidine-1-carboxamide;
3-(1-cyclohexyl-4-oxo-4,5-dihydro-1H-pyrazolo[3,4-d]pyrimidin-6-yl)-N-(4-morpholinophenyl)azetidine-1-carboxamide;
3-(1-cyclohexyl-4-oxo-4,5-dihydro-1H-pyrazolo[3,4-d]pyrimidin-6-yl)-N-phenylazetidine-1-carboxamide;
3-(1-tert-butyl-3-methyl-4-oxo-4,5-dihydro-1H-pyrazolo[3,4-d]pyrimidin-6-yl)-N-(4-fluorophenyl)azetidine-1-carboxamide;
3-(1-cyclohexyl-4-oxo-4,5-dihydro-1H-pyrazolo[3,4-d]pyrimidin-6-yl)-N-(4-methoxyphenyl)azetidine-1-carboxamide;
3-(1-cyclohexyl-3-methyl-4-oxo-4,5-dihydro-1H-pyrazolo[3,4-d]pyrimidin-6-yl)-N-phenylazetidine-1-carboxamide;
3-(1-tert-butyl-3-methyl-4-oxo-4,5-dihydro-1H-pyrazolo[3,4-d]pyrimidin-6-yl)-N-cyclohexylazetidine-1-carboxamide;
3-(1-tert-butyl-4-oxo-4,5-dihydro-1H-pyrazolo[3,4-d]pyrimidin-6-yl)-N-(4-fluorophenyl)azetidine-1-carboxamide;
3-(1-tert-butyl-3-methyl-4-oxo-4,5-dihydro-1H-pyrazolo[3,4-d]pyrimidin-6-yl)-N-(4-fluorobenzyl)azetidine-1-carboxamide;
3-(1-cyclohexyl-4-oxo-4,5-dihydro-1H-pyrazolo[3,4-d]pyrimidin-6-yl)-N-(4-isopropylphenyl)azetidine-1-carboxamide;
3-(1-cyclohexyl-4-oxo-4,5-dihydro-1H-pyrazolo[3,4-d]pyrimidin-6-yl)-N-(4-fluorophenyl)azetidine-1-carboxamide;
3-(1-cyclohexyl-4-oxo-4,5-dihydro-1H-pyrazolo[3,4-d]pyrimidin-6-yl)-N-(3,4-difluorophenyl)azetidine-1-carboxamide;
3-(1-cyclohexyl-3-methyl-4-oxo-4,5-dihydro-1H-pyrazolo[3,4-d]pyrimidin-6-yl)-N-(4-fluorophenyl)azetidine-1-carboxamide;
N-benzyl-3-(1-tert-butyl-3-methyl-4-oxo-4,5-dihydro-1H-pyrazolo[3,4-d]pyrimidin-6-yl)azetidine-1-carboxamide;
N-cyclohexyl-3-(1-cyclohexyl-4-oxo-4,5-dihydro-1H-pyrazolo[3,4-d]pyrimidin-6-yl)azetidine-1-carboxamide;
N-benzyl-3-(1-cyclohexyl-3-methyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(trifluoromethoxy)phenyl)azetidine-1-carboxamide;
tert-butyl 3-(3-(1-cyclohexyl-4-oxo-4,5-dihydro-1H-pyrazolo[3,4-d]pyrimidin-6-yl)azetidine-1-carboxamido)piperidine-1-carboxylate;
3-(1-cyclohexyl-3-methyl-4-oxo-4,5-dihydro-1H-pyrazolo[3,4-d]pyrimidin-6-yl)-N-(4-fluorobenzyl)azetidine-1-carboxamide;
3-(1-cyclohexyl-3-methyl-4-oxo-4,5-dihydro-1H-pyrazolo[3,4-d]pyrimidin-6-yl)-N-(4-methoxybenzyl)azetidine-1-carboxamide;
N-(2-(difluoromethoxy)phenyl)-3-(1-(4-fluorophenyl)-3-methyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-fluorobenzyl)azetidine-1-carboxamide;
N-benzyl-3-(1-cyclohexyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-cyclopentylazetidine-1-carboxamide;
N-(4-cyanophenyl)-3-(1-cyclohexyl-4-oxo-4,5-dihydro-1H-pyrazolo[3,4-d]pyrimidin-6-yl)azetidine-1-carboxamide;
N-butyl-3-(1-cyclohexyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(trifluoromethyl)phenyl)azetidine-1-carboxamide;
3-(1-cyclohexyl-4-oxo-4,5-dihydro-1H-pyrazolo[3,4-d]pyrimidin-6-yl)-N-(3,4-dichlorobenzyl)azetidine-1-carboxamide;
N-tert-butyl-3-(1-cyclohexyl-4-oxo-4,5-dihydro-1H-pyrazolo[3,4-d]pyrimidin-6-yl)azetidine-1-carboxamide;
3-(1-cyclohexyl-3-methyl-4-oxo-4,5-dihydro-1H-pyrazolo[3,4-d]pyrimidin-6-yl)-N-(3,4-dichlorobenzyl)azetidine-1-carboxamide;
3-(1-cyclohexyl-4-oxo-4,5-dihydro-1H-pyrazolo[3,4-d]pyrimidin-6-yl)-N-(1-(methylsulfonyl)piperidin-4-yl)azetidine-1-carboxamide;
tert-butyl 4-(3-(1-cyclohexyl-4-oxo-4,5-dihydro-1H-pyrazolo[3,4-d]pyrimidin-6-yl)azetidine-1-carboxamido)piperidine-1-carboxylate;
(S)-3-(1-cyclohexyl-3-methyl-4-oxo-4,5-dihydro-1H-pyrazolo[3,4-d]pyrimidin-6-yl)-N-(1-phenylethyl)azetidine-1-carboxamide; and
3-(1-cyclohexyl-4-oxo-4,5-dihydro-1H-pyrazolo[3,4-d]pyrimidin-6-yl)-N-(3,3,3-trifluoropropyl)azetidine-1-carboxamide; or a pharmaceutically acceptable salt thereof, and isotopic variants thereof, stereoisomers and tautomers thereof.

In another embodiment, with respect to compounds of formulae Ia-Ih, the compound is selected from:
1-cyclohexyl-6-(1-(3,4-dimethoxyphenylsulfonyl)piperidin-3-yl)-1H-pyrazolo[3,4-d]pyrimidin-4(5H)-one;
4-(4-isopropylpiperazin-1-yl)phenyl 3-(1-tert-butyl-4-oxo-4,5-dihydro-1H-pyrazolo[3,4-d]pyrimidin-6-yl)azetidine-1-carboxylate;
cyclohexylmethyl 3-(1-tert-butyl-4-oxo-4,5-dihydro-1H-pyrazolo[3,4-d]pyrimidin-6-yl)azetidine-1-carboxylate;
3-chlorophenyl 3-(1-tert-butyl-4-oxo-4,5-dihydro-1H-pyrazolo[3,4-d]pyrimidin-6-yl)azetidine-1-carboxylate;
benzo[d][1,3]dioxol-5-yl 3-(1-tert-butyl-4-oxo-4,5-dihydro-1H-pyrazolo[3,4-d]pyrimidin-6-yl)azetidine-1-carboxylate;
cyclohexylmethyl 3-(1-cyclohexyl-4-oxo-4,5-dihydro-1H-pyrazolo[3,4-d]pyrimidin-6-yl)azetidine-1-carboxylate;
4-fluorophenyl 3-(1-cyclohexyl-4-oxo-4,5-dihydro-1H-pyrazolo[3,4-d]pyrimidin-6-yl)azetidine-1-carboxylate;
6-(1-(1H-benzo[d]imidazol-2-yl)azetidin-3-yl)-1-cyclohexyl-1H-pyrazolo[3,4-d]pyrimidin-4(5H)-one;
6-(1-(6-bromo-1H-benzo[d]imidazol-2-yl)azetidin-3-yl)-1-tert-butyl-1H-pyrazolo[3,4-d]pyrimidin-4(5H)-one;
1-tert-butyl-6-(1-(5-(trifluoromethyl)-1H-benzo[d]imidazol-2-yl)azetidin-3-yl)-1H-pyrazolo[3,4-d]pyrimidin-4(5H)-one;
6-(1-(1H-benzo[d]imidazol-2-yl)azetidin-3-yl)-1-tert-butyl-1H-pyrazolo[3,4-d]pyrimidin-4(5H)-one;
1-cyclohexyl-6-(1-(4-phenyloxazol-2-yl)azetidin-3-yl)-1H-pyrazolo[3,4-d]pyrimidin-4(5H)-one;
1-cyclohexyl-6-(1-(4-(morpholinosulfonyl)phenyl)azetidin-3-yl)-1H-pyrazolo[3,4-d]pyrimidin-4(5H)-one;
6-(1-(3-amino-4-nitrophenyl)azetidin-3-yl)-1-cyclohexyl-1H-pyrazolo[3,4-d]pyrimidin-4(5H)-one;
6-(1-(4-acetoylphenyl) azetidin-3-yl)-1-cyclohexyl-1H-pyrazolo[3,4-d]pyrimidin-4(5H)-one;
4-(3-(1-cyclohexyl-4-oxo-4,5-dihydro-1H-pyrazolo[3,4-d]pyrimidin-6-yl)azetidin-1-yl)benzonitrile;
1-cyclohexyl-6-(1-(3-methyl-4-nitrophenyl)azetidin-3-yl)-1H-pyrazolo[3,4-d]pyrimidin-4(5H)-one;
1-cyclohexyl-6-(1-(4-(morpholinomethyl)phenyl)azetidin-3-yl)-1H-pyrazolo[3,4-d]pyrimidin-4(5H)-one;
1-cyclohexyl-6-(1-(5-phenyl-4H-1,2,4-triazol-3-yl)azetidin-3-yl)-1H-pyrazolo[3,4-d]pyrimidin-4(5H)-one;
1-cyclohexyl-6-(1-(4-phenylthiazol-2-yl)azetidin-3-yl)-1H-pyrazolo[3,4-d]pyrimidin-4(5H)-one;
1-cyclohexyl-6-(1-(4-(4-(4-isopropylpiperazin-1-yl)phenyl)thiazol-2-yl)azetidin-3-yl)-1H-pyrazolo[3,4-d]pyrimidin-4(5H)-one;
1-cyclohexyl-6-(1-(5-(morpholinomethyl)-4-phenylthiazol-2-yl)azetidin-3-yl)-1H-pyrazolo[3,4-d]pyrimidin-4(5H)-one;
1-tert-butyl-6-(1-(4-(morpholinomethyl)phenyl)azetidin-3-yl)-1H-pyrazolo[3,4-d]pyrimidin-4(5H)-one;
(Z)-N′-cyano-3-(1-cyclohexyl-4-oxo-4,5-dihydro-1H-pyrazolo[3,4-d]pyrimidin-6-yl)-N-(4-fluorophenyl)azetidine-1-carboximidamide;
(Z)-N′-cyano-3-(1-cyclohexyl-4-oxo-4,5-dihydro-1H-pyrazolo[3,4-d]pyrimidin-6-yl)-N-(cyclohexylmethyl)azetidine-1-carboximidamide; and
(Z)-3-(1-tert-butyl-4-oxo-4,5-dihydro-1H-pyrazolo[3,4-d]pyrimidin-6-yl)-N′-cyano-N-(4-fluorophenyl)azetidine-1-carboximidamide;
   or a pharmaceutically acceptable salt thereof, and isotopic variants thereof, stereoisomers and tautomers thereof.

In yet another embodiment, with respect to compounds of formulae Ia-Ih, the compound is selected from all compounds of the invention exemplified specifically herein.

A compound for use according to the invention may contain one or more asymmetric carbon atoms and may exist in racemic and optically active forms. It will be understood by a person of skill in the art that the present invention includes both the racemic mixture and each enantiomer in isolated form. A compound according to an embodiment of the invention may be in trans or cis form.

The present invention also extends to a prodrug of a compound according to an embodiment of the invention such as an ester or amide thereof. A prodrug is a compound that may be converted under physiological conditions or by solvolysis to a compound according to an embodiment of the invention or to a pharmaceutically acceptable salt of a compound according to an embodiment of the invention. A prodrug may be inactive when administered to a subject but is converted in vivo to an active compound of the invention. ‘Pharmaceutically acceptable prodrugs’ as used herein refers to those prodrugs of the compounds useful in the present invention, which are, within the scope of sound medical judgment, suitable for use in contact with the tissues of patients with undue toxicity, irritation, allergic response commensurate with a reasonable benefit/risk ratio, and effective for their intended use of the compounds of the invention. The term ‘prodrug’ means a compound that is transformed in vivo to yield an effective compound useful in the present invention or a pharmaceutically acceptable salt, hydrate or solvate thereof. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as prodrugs. A thorough discussion is provided in Design of Prodrugs, H. Bundgard, ed., Elsevier (1985); Methods in Enzymology; K. Widder et al, Ed., Academic Press, 42, 309-396 (1985); A Textbook of Drug Design and Development, Krogsgaard-Larsen and H. Bundgard, ed., Chapter 5; “Design and Applications of Prodrugs” 113-191 (1991); Advanced Drug Delivery Reviews, H. Bundgard, 8, 1-38, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, all of which are incorporated herein by reference.

### Pharmaceutical Compositions

Compounds of the invention can be incorporated into pharmaceutical compositions suitable for administration. Such compositions typically comprise at least one compound of the invention and at least one pharmaceutically acceptable carrier. As used herein the language ‘pharmaceutically acceptable carrier’ is intended to include solid carriers such as lactose, magnesium stearate, terra alba, sucrose, talc, stearic acid, gelatin, agar, pectin, acacia or the like; and liquids such as vegetable oils, arachis oil and sterile water, or the like, any and all solvents, dispersion media, coatings, antibacterial and antifungal agents, isotonic and absorption delaying agents, and the like, compatible with pharmaceutical administration. This listing of pharmaceutically acceptable carriers is not to be construed as limiting. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a compound according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

A compound according to an embodiment of the invention may be provided as a salt, preferably as a pharmaceutically acceptable salt of compounds of formula I or Formulae Ia-Ih. Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of ammonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases which are nontoxic and strong enough to form salts. Such organic bases are already well known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl)aminomethane; and the like.

Salts of compounds according to an embodiment of the invention may be prepared in a conventional manner using methods well known in the art. Acid addition salts of said basic compounds may be prepared by dissolving the free base compounds according to the first or second aspects of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g by evaporation. The compounds of this invention may also exist in solvated or hydrated forms.

The following formulation examples illustrate representative pharmaceutical compositions of this invention. The present invention, however, is not limited to the following pharmaceutical compositions.

### FORMULATION 1

### Tablets

A compound of the invention is admixed as a dry powder with a dry gelatin binder in an approximate 1:2 weight ratio. A minor amount of magnesium stearate is added as a lubricant. The mixture is formed into 240-270 mg tablets (80-90 mg of active amide compound per tablet) in a tablet press.

### FORMULATION 2

### Capsules

A compound of the invention is admixed as a dry powder with a starch diluent in an approximate 1:1 weight ratio. The mixture is filled into 250 mg capsules (125 mg of active amide compound per capsule).

### FORMULATION 3

### Liquid

A compound of the invention (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### FORMULATION 4

### Tablets

A compound of the invention is admixed as a dry powder with a dry gelatin binder in an approximate 1:2 weight ratio. A minor amount of magnesium stearate is added as a lubricant. The mixture is formed into 450-900 mg tablets (150-300 mg of active amide compound) in a tablet press.

### FORMULATION 5

### Injection

A compound of the invention is dissolved or suspended in a buffered sterile saline injectable aqueous medium to a concentration of approximately 5 mg/mL.

### FORMULATION 6

### Topical

Stearyl alcohol (250 g) and a white petrolatum (250 g) are melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

### Methods of Treatment

The present invention relates also to a method of treatment or prevention of osteoarthritis, which comprises administering to a subject in need thereof, a therapeutically effective amount of compound of the invention.

The present invention relates also to a method of treatment or prevention of osteoarthritis, which comprises administering to a subject in need thereof, a therapeutically effective amount of an inhibitor of PDE1A according to Formula I.

The present invention relates also to a method of treatment or prevention of osteoarthritis, which comprises administering to a subject in need thereof, a therapeutically effective amount of an inhibitor of PDE1A according to Formulae Ia-Ii.

Another aspect of the present method invention relates to a method of treatment or prophylaxis of a condition characterized by abnormal PDE1A activity, which comprises administering a therapeutically effective amount of a PDE1A inhibiting compound according to Formula I or Formulae Ia-Ih.

A further aspect of the present method invention is a method of treatment or prophylaxis of a disease involving degradation of cartilage, which comprises administering a therapeutically effective a compound according to Formula I or Formulae Ia-Ih.

A special embodiment of the present method invention is a method of treatment or prevention of OA, which comprises administering to a subject in need thereof, a therapeutically effective amount of a compound according to Formula I or Formulae Ia-Ih.

This invention also relates to the use of the present compounds in the manufacture of a medicament for treatment or prophylaxis of a condition prevented, ameliorated or eliminated by administration of an inhibitor of PDE1A which is a compound of the invention, or a condition selected from diseases involving inflammation, most preferably for the treatment of diseases selected from osteoarthritis, rheumatoid arthritis and osteoporosis.

Administration of the compound of the present invention to the subject patient includes both self-administration and administration by another person. The patient may be in need of treatment for an existing disease or medical condition, or may desire prophylactic treatment to prevent or reduce the risk for diseases and medical conditions affected by a disturbance in bone metabolism. The compound of the present invention may be delivered to the subject patient orally, transdermally, via inhalation, injection, nasally, rectally or via a sustained release formulation.

A preferred regimen of the present method comprises the administration to a subject in suffering from a disease condition characterized by a disturbance in bone and/or cartilage metabolism, of an effective PDE1A-inhibiting amount of a compound of the present invention for a period of time sufficient to reduce the abnormal levels of bone and/or cartilage degradation in the patient, and preferably terminate, the self-perpetuating processes responsible for said degradation. A special embodiment of the method comprises administering of an effective PDE1A inhibiting amount of a compound of the present invention to a subject patient suffering from or susceptible to the development of osteoarthritis, for a period of time sufficient to reduce or prevent, respectively, collagen and bone degradation in the joints of said patient, and preferably terminate, the self-perpetuating processes responsible for said degradation.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

A preferred therapeutically effective amount of the compound of the present invention to administer to a subject patient is about 0.1 mg/kg to about 10 mg/kg administered from once to three times a day. For example, an effective regimen of the present method may administer about 5 mg to about 1000 mg of said compound of the present invention from once to three times a day. It will be understood, however, that the specific dose level for any particular subject patient will depend upon a variety of factors including the age, body weight, general health, sex, diet, time of administration, route of administration, rate of excretion, drug combination and the severity of the particular inflammatory condition. A consideration of these factors is well within the purview of the ordinarily skilled clinician for the purpose of determining the therapeutically effective or prophylactically effective dosage amount needed to prevent, counter, or arrest the progress of the condition.

For the prevention and/or treatment of long-term conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound of the invention, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of a condition related to bone and/or cartilage degradation the compounds of this invention will be administered to a patient at risk for developing the condition, typically on the advice and under the supervision of a physician, at the dosage levels described above. Patients at risk for developing a particular condition generally include those who have been identified by genetic testing or screening to be particularly susceptible to developing said condition.

The compounds of this invention can be administered as the sole active agent or they can be administered in combination with other agents, including other compounds that demonstrate the same or a similar therapeutic activity and that are determined to safe and efficacious for such combined administration.

The present invention will now be described in detail with reference to specific examples of compounds and methods for their production. Within this specification embodiments have been described in a way that enables a clear and concise specification to be written, but it will be appreciated that embodiments may be variously combined or separated without parting from the invention.

### EXAMPLES

### 1. Synthetic Preparation of Compounds of the Invention

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; however, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of representative compounds that have been listed hereinabove. The compounds of the invention may be prepared from known or commercially available starting materials and reagents by one skilled in the art of organic synthesis.

A compound according to the present invention can be produced according to the following scheme.

1.1. General Synthetic Route

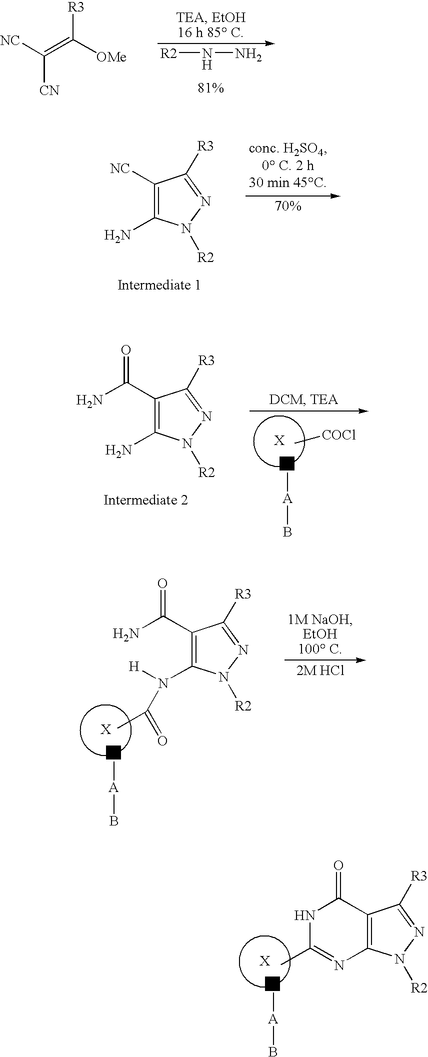

### Description 1: 5-Amino-1-cyclohexyl-1H-pyrazole-4-carboxylic Acid Amide (Intermediate 2A; R2=Cyclohexyl R3=H)

### Step 1: 5-Amino-1-cyclohexyl-1H-pyrazole-4-carbonitrile (Intermediate 1A; R2=Cyclohexyl, R3=H)

A solution of (2-ethoxymethylidene)malononitrile (10.35 g, 84.7 mmol), cyclohexylhydrazine hydrochloride (15.2 g, 100.9 mmol) and triethylamine (41 mL, 296.6 mmol) in EtOH (350 mL) was heated at 85° C. for 18 h. The ethanol was evaporated and the residue partitioned between diethyl ether (150 mL) and water (50 mL). The aqueous phase was extracted with diethyl ether (2×50 mL). Organic phases were combined, washed with brine (100 mL), dried (MgSO₄) and evaporated. The resulting solid was triturated with diethyl ether and petroleum ether 40-60° C. (1:20) and the solid was collected by filtration. 5-Amino-1-cyclohexyl-1H-pyrazole-4-carbonitrile (14.0 g, 87%) was isolated as a yellow solid that was used without any further purification. ¹H NMR (400 MHz, CDCl₃): δ 7.53 (1H, s), 4.56 (2H, s), 3.87-3.69 (1H, m), 1.92-1.66 (6H, m), 1.63-1.60 (1H, m), 1.46-1.29 (3H, m).

### Step 2: 5-Amino-1-cyclohexyl-1H-pyrazole-4-carboxylic Acid Amide

Concentrated sulfuric acid (98%, 100 mL) was cooled to 0-5° C., and 5-amino-1-cyclohexyl-1H-pyrazole-4-carbonitrile (14.0 g, 73.6 mmol) was added portionwise with vigorous stirring whilst maintaining the temperature below 10° C. The mixture was stirred at 0-5° C. for 2 h, then allowed to warm to 25° C. and stirred at this temperature for 1 h. The mixture was poured onto crushed ice and basified to pH 8-9 by cautious addition of ammonium hydroxide solution. The precipitate was collected by filtration and the filtrate was extracted with EtOAc (4×150 mL). The combined organic phases were washed with brine (100 mL), dried (MgSO₄) and concentrated to dryness. The resulting solid was mixed with the previously obtained precipitate and the mixture was triturated with diethyl ether and petroleum ether 40-60° C. (1:20) and the solid was collected by filtration. The title compound was isolated as a yellow solid (11.0 g, 72%) that was used without further purification. ¹H NMR (400 MHz, d₆-DMSO): δ 7.65 (1H, s), 7.16 (2H, br s), 6.19 (2H, s), 4.06-3.98 (1H, m), 1.83-1.66 (7H, m), 1.42-1.33 (2H, m), 1.23-1.14 (1H, m).

### Description 2: 5-Amino-1-tert-butyl-3-methyl-1H-pyrazole-4-carboxylic Acid Amide (Intermediate 2B: R2=t-Bu R3=Me)

### Step 1: 5-Amino-1-tert-butyl-3-methyl-1H-pyrazole-4-carbonitrile (Intermediate 1B; R2=t-Bu, R3=Me)

A solution of (1-ethoxyethylidene)malononitrile (25 g, 0.18 mol), tert butyl hydrazine hydrochloride (27.4 g, 0.22 mol) and triethylamine (103 mL, 0.71 mmol) in EtOH (600 mL) was heated at 85° C. for 16 h. After this time the EtOH was evaporated, and the residue partitioned between diethyl ether (500 mL) and water (300 mL). The aqueous layer was extracted with ether (500 mL) and the combined organic layers washed with brine (250 mL). The organic layer was dried over MgSO₄ and evaporated to give a pale yellow solid (21.4 g). This solid was slurried in ether/petroleum ether, filtered and dried to give a while solid (21.2 g). The original aqueous layer and brine wash were re-extracted with ether to give additional white solid (7.2 g). The two batches of solid were combined to give the title compound as a white solid (28.4 g, 89%) which was used without further purification. ¹H NMR (400 MHz, CDCl₃): 4.23 (2H, s), 2.22 (3H, s), 1.60 (9H, s).

### Step 2: 5-Amino-1-tert-butyl-3-methyl-1H-pyrazole-4-carboxylic Acid Amide

A solution of 5-amino-1-tert-butyl-3-methyl-1H-pyrazole-4-carbonitrile (14.1 g, 79.1 mmol) in a mixture of EtOH (90 mL) and DMSO (22.5 mL) was cooled to 0° C. Sodium hydroxide solution (1N, 26 mL) was added followed by dropwise addition of hydrogen peroxide solution (35 wt. % in water, 10.5 mL, 118.6 mmol). The reaction was maintained at 0° C. for 10 min then allowed to warm to room temperature. An exotherm was observed when the reaction reached the ambient temperature. Once the reaction had cooled to room temperature, EtOH was removed under reduced pressure and the residue was diluted with water (100 mL) causing the formation of a precipitate. The precipitate was collected by filtration, rinsed with water and dried. The filtrate was extracted with EtOAc (3×150 mL). The combined organic phases were washed with water (100 mL) then with brine (2×100 mL), dried (MgSO4) and concentrated to dryness. The resulting solid was mixed with the previously obtained precipitate and the mixture was triturated with diethyl ether and petroleum ether 40-60° C. (1:20) and the solid was collected by filtration. 5-Amino-1-tert-butyl-3-methyl-1H-pyrazole-4-carboxylic acid amide (13.7 g, 88%) was isolated as white solid that was used without any further purification. ¹H NMR (400 MHz, d₆-DMSO): δ 6.51 (2H, br s), 6.19 (2H, s), 2.23 (3H, s), 1.52 (9H, s).

### Description 3: 6-Azetidin-3-yl-1-tert-butyl-3-methyl-1,5-dihydropyrazolo[3,4-d]pyrimidin-4-one Hydrochloride

### Step 1: 1-Benzhydrylazetidine-3-carbonyl Chloride Hydrochloride

1-Benzhydrylazetidine-3-carboxylic acid (16.0 g, 59.8 mmol) was added portionwise to thionyl chloride (50 mL), and the resulting mixture was stirred at room temperature for 4 h. Thionyl chloride was then removed under reduced pressure. The residue was triturated with THF (50 ml) which was then evaporated. 1-Benzhydrylazetidine-3-carbonyl chloride hydrochloride (19.3 g, 100%) was isolated as a beige solid, and used without further purification.

### Step 2: 6-(1-Benzhydrylazetidin-3-yl)-1-tert-butyl-3-methyl-1,5-dihydropyrazolo[3,4-d]pyrimidin-4-one

Pyridine (9.7 mL, 119.2 mmol) was added to a solution of 5-amino-1-tert-butyl-3-methyl-1H-pyrazole-4-carboxylic acid amide (Description 2, 7.8 g, 39.7 mmol), 1-benzhydrylazetidine-3-carbonyl chloride hydrochloride (19.3 g, 59.8 mmol) and 4-dimethylaminopyridine (488 mg, 4.0 mmol) in DCM (300 mL). The resulting mixture was heated at 50° C. for 18 h. After cooling to room temperature, the reaction was washed with 1M sodium carbonate solution (3×100 mL). The aqueous phases were combined and extracted with DCM (100 mL). The organic phases were combined, washed with brine (100 mL), dried (MgSO₄) and evaporated. The residue was then dissolved in EtOH (400 mL). Sodium hydroxide solution (1N, 200 mL, 198.7 mmol) was added and the reaction was heated at 100° C. for 16 h. Ethanol was then removed under reduced pressure and hydrochloric acid solution (1N) was added until pH 5-6. A white precipitate formed and was collected by filtration. The solid was triturated with diethyl ether and petroleum ether 40-60° C. (1:20), filtered and dried to give 6-(1-benzhydrylazetidin-3-yl)-1-tertbutyl-3-methyl-1,5-dihydropyrazolo[3,4-d]pyrimidin-4-one as a white solid (6.0 g, 35%) that was used without further purification. ¹H NMR (400 MHz, d₆-DMSO): δ 11.93 (1H, s), 7.47-7.46 (4H, m), 7.33-7.29 (4H, m), 7.23-7.20 (2H, m), 4.50 (1H, s), 3.69-3.66 (1H, m), 3.50-3.46 (2H, m), 3.33-3.29 (2H, m), 2.43 (3H, s), 1.74 (9H, s).

### Step 3: 6-Azetidin-3-yl-1-tert-butyl-3-methyl-1,5-dihydro-pyrazolo[3,4-d]pyrimidin-4-one Hydrochloride

A suspension of 6-(1-benzhydrylazetidin-3-yl)-1-tert-butyl-3-methyl-1,5-dihydropyrazolo[3,4-d]pyrimidin-4-one (5.0 g, 11.7 mmol) and palladium hydroxide (20 wt. %, 5.0 g) in a mixture of EtOH (65 mL) and hydrochloric acid (1N, 32.5 mL) was stirred at room temperature for 18 h under hydrogen (200 psi). Catalyst was removed by filtered through celite and the filtrate evaporated under reduced pressure. The residue was partitioned between DCM (100 mL) and water (150 mL). The aqueous phase was collected and evaporated to dryness. The residue was triturated with diethyl ether and petroleum ether 40-60° C. (1:20), filtered and dried to give 6-azetidin-3-yl-1-tert-butyl-3-methyl-1,5-dihydro-pyrazolo[3,4-d]pyrimidin-4-one hydrochloride, which was isolated as white solid (3.0 g, 86%) and used without any further purification. ¹H NMR (400 MHz, d₆-DMSO): δ 12.10 (1H, s), 9.56 (1H, s), 9.17 (1H, s), 4.30-4.27 (2H, m), 4.18-4.15 (3H, m), 2.43 (3H, s), 1.73 (9H, s). MS (MH⁺, m/z) 262.

### Description 4: 6-Azetidin-3-yl-1-cyclohexyl-1,5-dihydropyrazolo[3,4-d]pyrimidin-4-one hydrochloride

The title compound was prepared from Description 1 according to the procedure of Description 3.

¹H NMR (400 MHz, d₆-DMSO): δ 12.19 (1H, s), 9.62 (1H, s), 9.11 (1H, s), 8.07 (1H, s), 4.70-4.63 (1H, m), 4.38-4.32 (2H, m), 4.21-4.05 (3H, m), 2.00-1.88 (6H, m), 1.75-1.72 (1H, m), 1.49-1.44 (2H, m), 1.12-1.09 (1H, m). MS (MH⁺, m/z) 274 (parent).

### Description 5: 1-Cyclohexyl-3-methyl-6-piperidin-3-yl-1,5-dihydro-pyrazolo[3,4-d]pyrimidin-4-one Hydrochloride

### Step 1: 1-Benzylpiperidine-3-carbonyl Chloride Hydrochloride

Hydrochloric acid (20% aq, 100 mL) was added to ethyl 1-benzylpiperidine-3-carboxylate (14.2 g, 57.4 mmol) and the mixture heated at reflux for 4 h. The reaction was cooled and concentrated in vacuo to give 1-benzylpiperidine-3-carboxylic acid as a pale yellow solid. This solid was dissolved in thionyl chloride and the resulting solution stirred at room temperature for 1 h. Thionyl chloride was removed in vacuo and the resulting solid was slurried in THF and azeotroped to afford the title compound as a pale yellow solid which was used without further purification (17.0 g, quant.).

### Step 2: 1-Cyclohexyl-3-methyl-6-piperidin-3-yl-1,5-dihydro-pyrazolo[3,4-d]pyrimidin-4-one Hydrochloride

The title compound was prepared from the product of Step 1 using the procedures described in Description 3, Step 2 and Step 3. ¹H NMR (400 MHz, CDCl₃): δ 11.50 (1H, br s), 4.52 (1H, m), 3.29 (1H, dd, J=12, 4 Hz), 3.10 (2H, m), 2.91 (1H, m), 2.81 (1H, m), 2.58 (3H, s), 2.04-1.88 (8H, m), 1.73 (2H, m), 1.60 (1H, m), 1.44 (2H, m), 1.31 (1H, m). MS (MH⁺, m/z) 316.

Descriptions 6-14 in Table 1 were prepared from (1-ethoxymethylidene)malononitrile, (1-ethoxyethylidene)malononitrile, (1-ethoxyproylidene)malononitrile (U.S. Pat. No. 5,541,187) and commercially available hydrazines according to Description 3, 4 or 5.

TABLE 1

| De- | | MS |
|---|---|---|
| scrip- | | (MH⁺, |
| tion- | Name | m/z) |
| 6 | 6-Azetidin-3-yl-1-tert-butyl-1,5-dihydro-pyrazolo[3,4- | 248 |
| | d]pyrimidin-4-one hydrochloride | |
| 7 | 6-Azetidin-3-yl-1-cyclohexyl-3-methyl-1,5-dihydro- | 288 |
| | pyrazolo[3,4-d]pyrimidin-4-one hydrochloride | |
| 8 | 6-Azetidin-3-yl-1-(4-fluoro-phenyl)-3-methyl-1,5- | 300 |
| | dihydro-pyrazolo[3,4-d]pyrimidin-4-one hydrochloride | |
| 9 | 1-Cyclobutyl-3-methyl-6-piperidin-3-yl-1,5-dihydro- | 288 |
| | pyrazolo[3,4-d]pyrimidin-4-one hydrochloride | |
| 10 | 1-Cyclohexyl-6-piperidin-3-yl-1,5-dihydro-pyrazolo[3,4- | 302 |
| | d]pyrimidin-4-one hydrochloride | |
| 11 | 1-Phenyl-6-piperidin-3-yl-1,5-dihydro-pyrazolo[3,4- | 296 |
| | d]pyrimidin-4-one hydrochloride | |
| 12 | 3-Methyl-1-phenyl-6-piperidin-3-yl-1,5-dihydro- | 310 |
| | pyrazolo[3,4-d]pyrimidin-4-one hydrochloride | |
| 13 | 3-Methyl-6-piperidin-3-yl-1-propyl-1,5-dihydro- | 278 |
| | pyrazolo[3,4-d]pyrimidin-4-one hydrochloride | |
| 14 | 6-Azetidin-3-yl-1-cyclohexyl-3-ethyl-1,5-dihydro- | 302 |
| | pyrazolo[3,4-d]pyrimidin-4-one hydrochloride | |

### Description 15: 4-(4-Isopropyl-piperazin-1-yl)-phenylamine

4-(piperazin-1-yl)-nitrobenzene (5.00 g, 24.2 mmol) was dissolved in acetone (10 mL) and MeOH (15 mL). AcOH (0.3 mL) then NaCNBH₃ (3.03 g, 48.4 mmol) were then added and the mixture was stirred for 26 h. The solvents were removed in vacuo and the residue was dissolved in DCM (20 mL). The organics were washed with 1M NaOH solution (20 mL), dried over MgSO₄ and concentrated in vacuo to give a yellow solid. This solid was dissolved in EtOAc (125 mL) and SnCl₂.2H₂O (27.30 g, 121 mmol) was added. The orange suspension was heated at reflux for 1.5 h then cooled to room temperature. Na₂CO₃ (1M, 150 mL) and water (150 mL) were added and the mixture was filtered. The organic layer was isolated and the aqueous layer was extracted with EtOAc (3×150 ml). The combined organics were dried over MgSO₄ and concentrated in vacuo to give an orange oil. This was re-dissolved in a minimum volume of EtOAc and hexane was added to induce crystallization. Filtration gave the required product as a yellow solid (2.34 g, 44%). ¹H NMR (400 MHz, CDCl₃): δ 6.83-6.79 (2H, m), 6.66-6.64 (2H, m), 3.09-2.98 (4H, m), 2.75-2.65 (5H, m), 1.09 (6H, d, J=6.8). MS (MH⁺, m/z) 220.

### Description 16: 3-(4-Isopropyl-piperazin-1-yl)-phenylamine

3-(piperazin-1-yl)-nitrobenzene hydrochloride (5.00 g, 20.6 mmol) was dissolved in MeOH (15 mL). Triethylamine (2.86 mL, 20.6 mmol) was added and the mixture was stirred for 5 min. Acetone (10 mL), AcOH (0.3 mL) then NaCNBH₃ (3.03 g, 48.4 mmol) were then added and the mixture was stirred for 16 h. The solvents were removed in vacuo and the residue was dissolved in DCM (20 mL). The organics were washed with NaOH solution (1M, 20 mL), dried over MgSO₄ and concentrated in vacuo to give a brown oil. This was dissolved in EtOH (35 mL) and EtOAc (35 mL) and the system was purged with N₂. Palladium on carbon (10%, 0.050 g) and AcOH (0.5 ml) were added and the system was stirred under a hydrogen atmosphere (10 bar) for 16 h. The catalyst was removed by filtration over kieselguhir then the solvents were removed in vacuo. The residue was dissolved in MeOH (25 mL) HCl (1M, 50 mL) then evaporated to give the product as the dihydrochloride salt (purple solid, 4.87 g, 81%). ¹H NMR (400 MHz, CDCl₃): δ 7.04 (1H, t, J=8), 6.36 (1H, d, J=8), 6.26-6.21 (2H, m), 3.23-3.21 (4H, m), 2.85-2.72 (5H, m), 1.12 (6H, d; J=6.4). MS (MH⁺, m/z) 220.

### Description 17: 4-(4-Isopropyl-piperazin-1-yl)-phenol

4-(piperazin-1-yl)-phenol (1.00 g, 5.62 mmol) was suspended in MeOH (3 mL) and acetone (2 mL) and AcOH (0.10 mL) was added. The mixture was stirred at 60° C. for 30 min then NaCNBH₃ (0.71 g, 11.24 mmol) was added. The mixture was stirred at 60° C. for 16 h then the solvents were removed in vacuo. The residue was dissolved in water (10 mL) and HCl (1M, 5.62 mL, 5.62 mmol) was added. The aqueous was extracted with EtOAc (5×10 mL). The combined organics were dried over MgSO₄ then concentrated in vacuo to give the product as a white solid (1.03 g, 83%). ¹H NMR (400 MHz, d6-DMSO): δ 8.79 (1H, brs), 7.69-7.67 (2H, m), 6.54-6.51 (2H, m), 3.05-2.50 (9H, m), 1.05-0.91 (6H, m). MS (MH⁺, m/z) 221.

### Description 18: 1-[4-(4-Hydroxyphenyl)-piperazin-1-yl]-ethanone

4-(piperazin-1-yl)-phenol (1.00 g, 5.62 mmol) was dissolved in pyridine (10 mL) and cooled to 0° C. Ac₂O (1.06, mL, 11.24 mmol) was added dropwise over 5 min with stirring. The mixture was allowed to warm to room temperature and stirring was continued for 3 h. The solvents were removed in vacuo to give a white solid that was dissolved in MeOH (20 mL) and water (2 mL). K₂CO₃ (1.55 g, 11.24 mmol) was added and the mixture was stirred for 1.5 h. The solvents were removed in vacuo and the residue was dissolved in DCM (20 mL). HCl (1M) was added to ca. pH 3. The organics were separated and the aqueous was extracted with DCM (4×10 mL). The combined organics were dried over MgSO₄ and concentrated in vacuo to give product as a pale pink solid. Further product was obtained after overnight crystallization from the aqueous layer. The combined solids were dried under high vacuum at 40° C. (0.67 g, 54%). ¹H NMR (400 MHz, d6-DMSO): δ 7.40-7.25 (2H, m), 6.85-6.83 (2H, m), 3.90-3.71 (4H, m), 3.35-3.20 (4H, m), 2.10 (3H, s). MS (MH⁺, m/z) 221.

### Description 19: 5-Bromo-1H-benzoimidazole-2-sulfonic Acid

Thiocarbonyl diimidazole (0.95 g, 5.35 mmol) was added in one portion to a solution of 4-bromobenzene-1,2-diamine (1.00 g, 2.67 mmol) in THF (50 mL). The mixture was stirred for 6 h then the solvents were removed in vacuo. The residue was suspended in DCM and the product was collected by filtration as a white solid. This was dissolved in KOH solution (1M, 10.7 mL, 10.7 mmol) and H₂O₂ (30%, 2.13 mL, 21.40 mmol) was added dropwise. The mixture was stirred for 14 h then concentrated HCl was added to pH 1. The mixture was cooled to 0° C. for 30 min then the white solid product was collected by filtration. This was dried at 40° C. under high vacuum (1.15 g, 77%). MS (MH⁺, m/z) 275 (50%), 277 (50%).

### Description 20: 5-(Trifluoromethyl)-1H-benzoimidazole-2-sulfonic Acid

Prepared from 4-(trifluoromethyl)-benzene-1,2-diamine according to the procedure of Description 19 to give the title product as a white solid (0.83 mmol, 0.22 g, 49%). MS (MH⁺, m/z) 267.

### Description 21: 5-(4-Methyl-piperazin-1-yl)-1H-benzoimidazole-2-sulfonic Acid

### Step 1: 4-(4-Methyl-piperazin-1-yl)-benzene-1,2-diamine

4-Methyl-piperazine (58 mmol, 6.40 ml), 5-chloro-2-nitro-phenylamine (5.8 mmol, 1.00 g), K₂CO₃ (29 mmol, 4.00 g) and DMF (15 mL) were heated in a sealed tube under N₂ at 160° C. for 21 h. The mixture was cooled and water (20 ml) was added. The mixture was extracted with EtOAc (4×20 mL). The combined organics were extracted with 1M HCl (50 mL). The aqueous layer was then basified (2M NaOH) then the precipitate formed was collected by filtration and dried under vacuum. This was dissolved in EtOH (25 mL) and EtOAc (25 mL) and placed under a N₂ atmosphere. Palladium on carbon (10% wt, 0.05 g) was added and the mixture was stirred under a H₂ atmosphere (3 bar) for 16 h. The catalyst was removed by filtration and the solvents were removed in vacuo to give the product as a dark solid (5.29 mmol, 1.09 g, 91%). ¹H NMR (400 MHz, CDCl₃): δ 6.64 (1H, d, J=12.4 Hz), 6.37 (1H, s), 6.34-6.32 (1H, m), 3.45 (2H, br s), 3.09-3.06 (4H, m), 2.58-2.56 (4H, m), 2.34 (3H, s). MS (MH⁺, m/z) 207.

### Step 2: 5-(4-Methyl-piperazin-1-yl)-1H-benzoimidazole-2-sulfonic Acid

Prepared from 4-(4-methyl-piperazin-1-yl)-benzene-1,2-diamine (Step 1) according to the procedure of Description 19 (beige solid, 2.09 mmol, 0.62 g, 40%). MS (MH⁺, m/z) 297.

### Description 22: 4-(4-Bromobenzyl)-morpholine

4-(4-Bromobenzyl)-morpholine was prepared from morpholine and 4-bromobenzaldehyde according to the procedure of Description 17 (1.60 mmol, 0.41 g, 80%). ¹H NMR (400 MHz, CDCl₃): δ 7.45-7.43 (2H, m), 7.24-7.20 (2H, m), 3.71-3.69 (4H, m), 3.44 (2H, s), 2.43-2.41 (4H, m). MS (MH⁺, m/z) 255 (50%) 257 (50%).

### Description 23: 4-(4-Fluoro-benzenesulfonyl)-morpholine

Morpholine (0.83 mL, 9.56 mmol) was added to a solution of 4-fluoro-benzenesulfonyl chloride (0.62 g, 3.19 mmol) in DCM (5 mL) and the reaction stirred for 4 h. The reaction was diluted with DCM, washed twice with saturated sodium bicarbonate solution, dried over MgSO₄ and the solvent evaporated to give the title compound, which was used without purification. ¹H NMR (400 MHz, CDCl₃): δ 7.78 (2H, m), 7.24 (2H, m), 3.73 (4H, m), 3.00 (4H, t, J=4.8).

3-Methanesulfonylamino-benzoyl chloride and 3-butanesulfonylamino-benzoyl chloride were prepared from the corresponding carboxylic acids using the procedure of Description 3, Step 1. The carboxylic acids were made in a sequence analogous to Example 181, Steps 1-2.

### Specific Examples

### Example 1

### N-(Benzo[d][1,3]dioxol-5-yl)-3-(1-tert-butyl-3-methyl-4-oxo-4,5-dihydro-1H-pyrazolo[3,4-d]pyrimidin-6-yl)azetidine-1-carboxamide

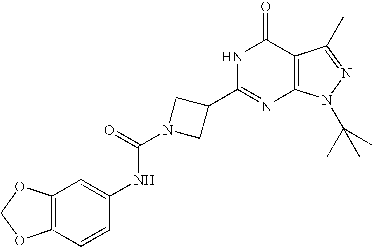

A suspension of Description 3 (60 mg, 0.21 mmol), morpholinomethyl PS resin (4.2 mmol/g, 100 mg) and 3,4-(methylenedioxy)phenyl isocyanate (39 mg) in DCM (4 mL) was shaken at room temperature for 16 h. Tris-(2-aminoethyl)-amine PS (100 mg) and methyl isocyanate PS (100 mg) scavenger resins were added and the mixture shaken for 5 h. The reaction was diluted with MeOH (˜4 mL), the resins removed by filtration and the filtrate evaporated. The crude product was purified by gradient column chromatography, eluting with 2-5% MeOH in DCM to give the title product as a white solid (43 mg, 51%). ¹H NMR (400 MHz, d6-DMSO) δ 12.08 (1H, br s), 8.48 (1H, s), 7.22 (1H, s), 6.90 (1H, d, J=8 Hz), 6.82 (1H, d, J=8 Hz), 5.99 (2H, s), 4.23 (4H, m), 3.86 (1H, m), 2.37 (3H, s), 1.71 (9H, s). MS (MH⁺, m/z) 425.

### Example 2

### 3-(1-Cyclohexyl-4-oxo-4,5-dihydro-1H-pyrazolo[3,4-d]pyrimidin-6-yl)-N-(4-(4-isopropylpiperazin-1-yl)phenyl)azetidine-1-carboxamide

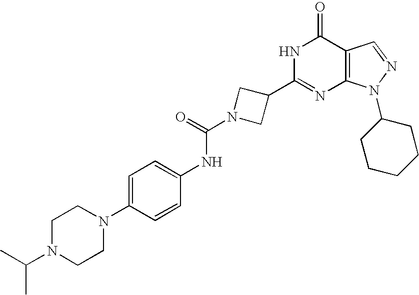

4-(4-Isopropyl-piperazin-1-yl)-phenylamine (Description 15, 53 mg, 0.24 mmol) was added to a solution of carbonyl diimidazole (39 mg, 0.24 mmol) in DCM (2 mL). The solution was stirred for 1 h then triethylamine (68 μL, 0.48 mmol) was added followed by Description 4 (75 mg, 0.24 mmol). The mixture was stirred for 16 h then diluted with DCM (2 mL) and washed with water (4 mL). The solvents were removed in vacuo and the residue was purified by gradient column chromatography eluting with 5-10% MeOH in DCM to give the title compound (37 mg, 29%). ¹H NMR (400 MHz, d6-DMSO): δ 12.15 (1H, s), 8.34 (1H, s), 8.05 (1H, s), 7.36 (2H, d, J=8), 6.86 (2H, d, J=8), 4.59 (1H, m), 4.24 (4H, m), 3.93 (1H, m), 3.10-3.00 (4H, m), 2.75-2.70 (1H, m), 2.65-2.60 (4H, m), 1.96-1.85 (6H, m), 1.71 (1H, m), 1.48-1.41 (2H, m), 1.28 (1H, m), 1.02 (6H, d, J=8). MS (MH⁺, m/z) 519 (parent).

Examples 3-114 listed in Table 2 were made from Descriptions 3-14 according to the procedure described in Example 1, using the appropriate isocyanate, isothiocyanate, acid chloride or sulfonyl chloride or according to the procedure described in Example 2 using the appropriate amine.

TABLE 2

| | | | MS |
|---|---|---|---|
| EXAMPLE | | | (MH⁺, |
| # | STRUCTURE | MW | m/z) |
| 3 | 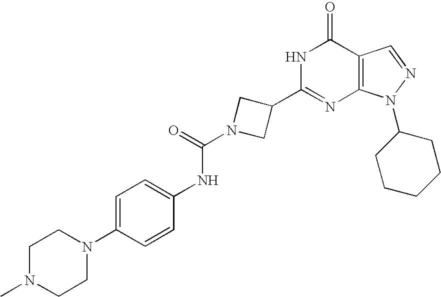 | 490.61 | 491 |
| 4 | 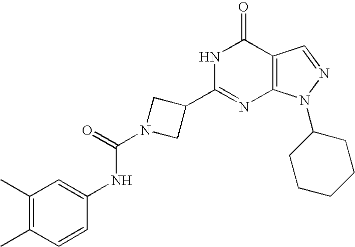 | 420.52 | 421 |
| 5 | 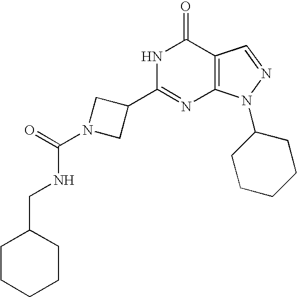 | 412.54 | 413 |
| 6 | 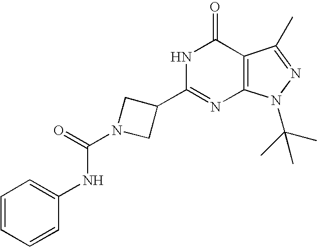 | 380.45 | 381 |
| 7 | 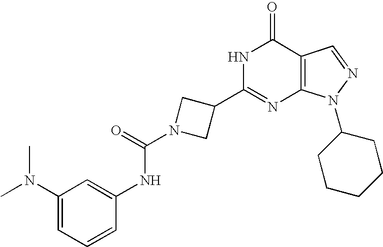 | 435.53 | 436 |
| 8 | 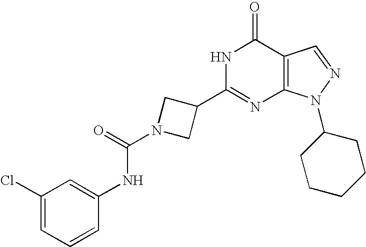 | 426.91 | 427 |
| 9 | 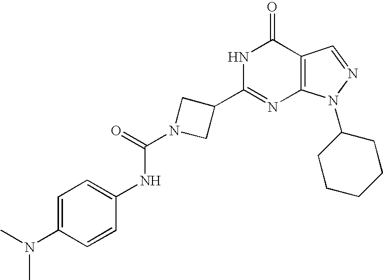 | 435.53 | 436 |
| 10 | 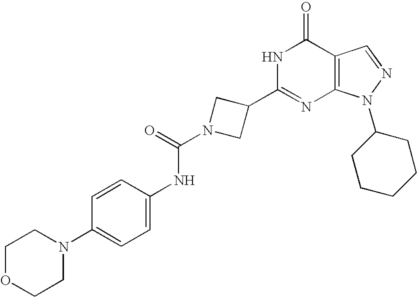 | 477.57 | 478 |
| 11 | 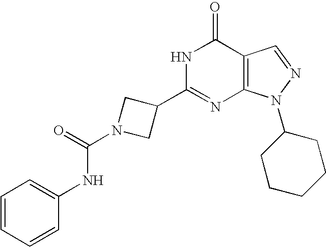 | 392.46 | 393 |
| 12 | 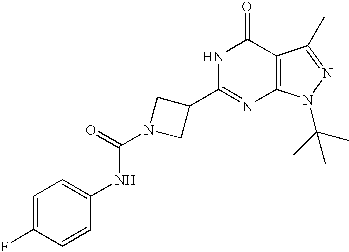 | 398.44 | 399 |
| 13 | 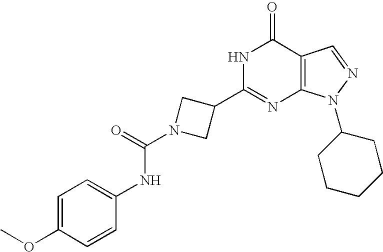 | 422.49 | 423 |
| 14 | 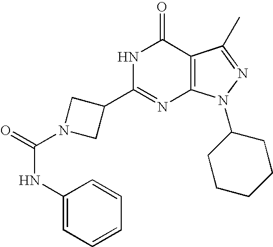 | 406.49 | 407 |
| 15 | 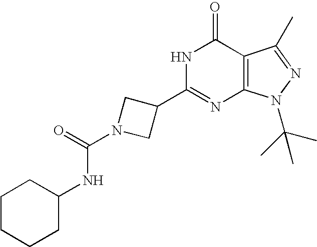 | 386.50 | 387 |
| 16 | 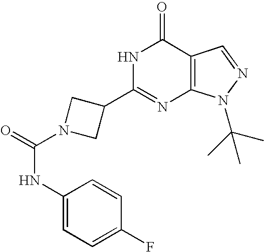 | 384.42 | 385 |
| 17 | 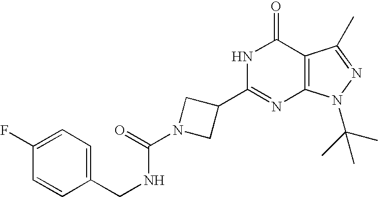 | 412.47 | 413 |
| 18 | 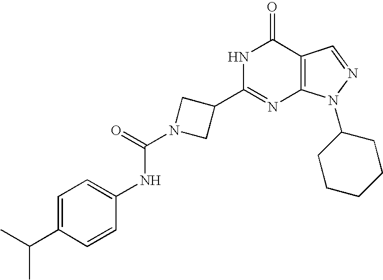 | 434.55 | 435 |
| 19 | 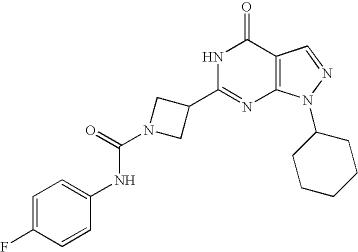 | 410.45 | 411 |
| 20 | 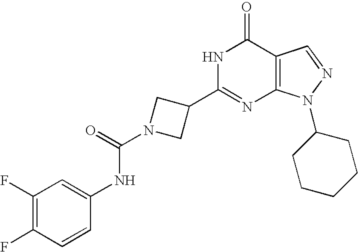 | 428.45 | 429 |
| 21 | 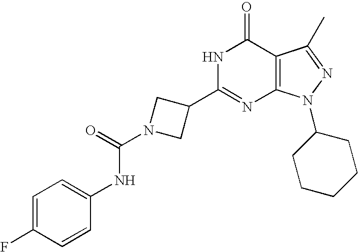 | 424.48 | 425 |
| 22 | 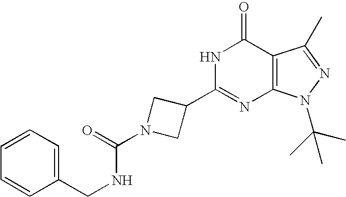 | 394.48 | 395 |
| 23 | 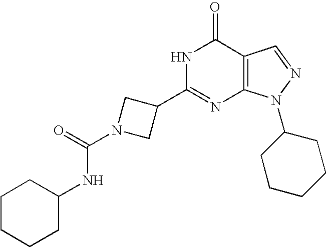 | 398.51 | 399 |
| 24 | 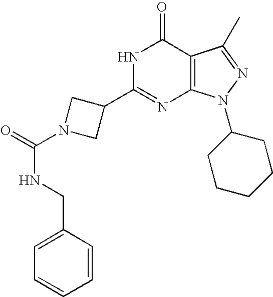 | 420.52 | 421 |
| 25 | 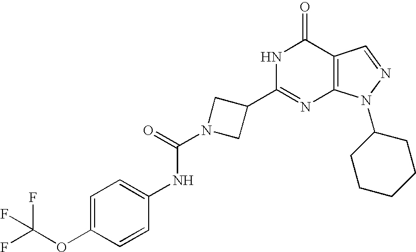 | 476.46 | 477 |
| 26 | 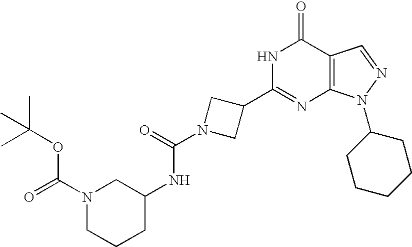 | 499.62 | 500 |
| 27 | 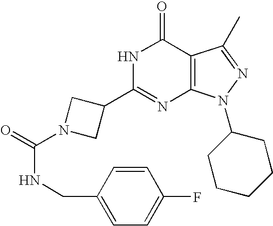 | 438.51 | 439 |
| 28 | 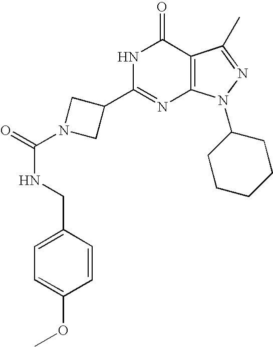 | 450.55 | 451 |
| 29 | 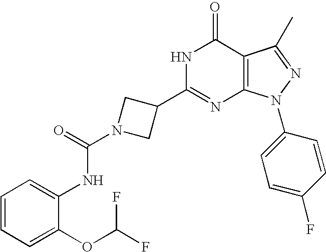 | 484.44 | 485 |
| 30 | 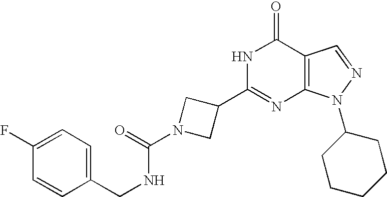 | 424.48 | 425 |
| 31 | 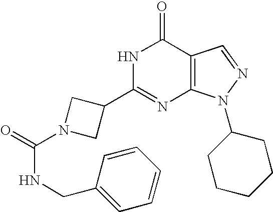 | 406.49 | 407 |
| 32 | 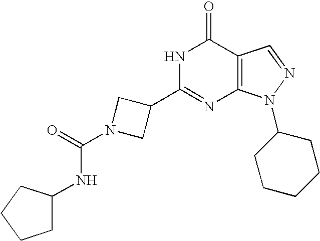 | 384.49 | 385 |
| 33 | 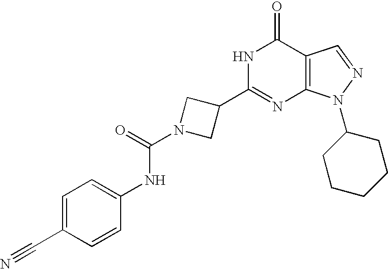 | 417.47 | 418 |
| 34 | 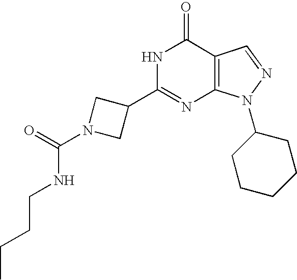 | 372.47 | 373 |
| 35 | 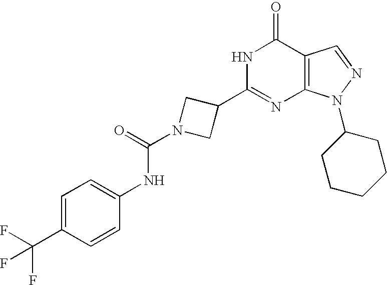 | 460.46 | 461 |
| 37 | 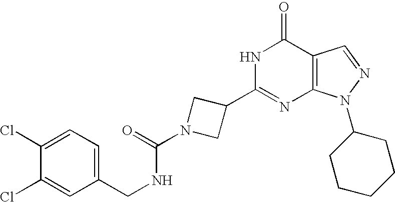 | 475.38 | 475, 477 |
| 38 | 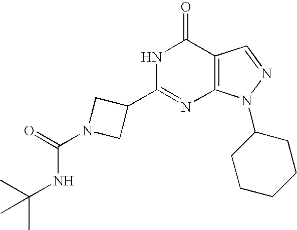 | 372.47 | 373 |
| 39 | 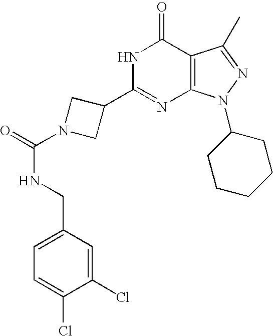 | 489.41 | 489, 491 |
| 40 | 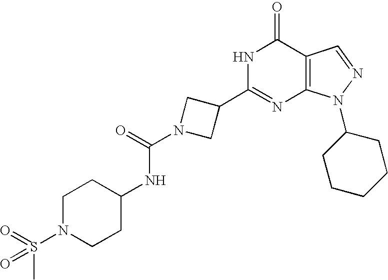 | 477.59 | 478 |
| 41 | 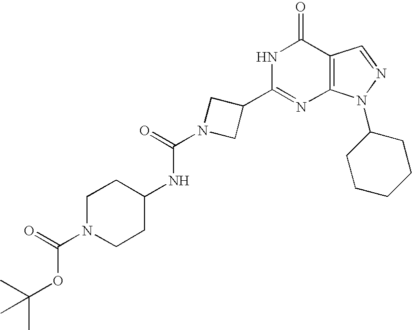 | 499.62 | 500.4 |
| 42 | 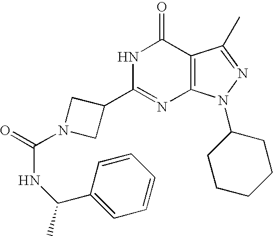 | 434.55 | 435 |
| 43 | 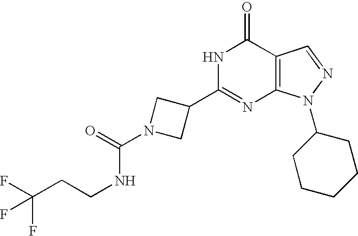 | 412.42 | 413 |
| 44 | 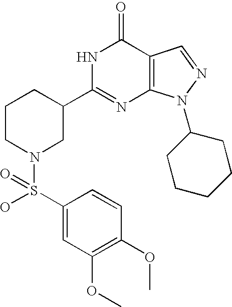 | 501.61 | 502 |
| 45 | 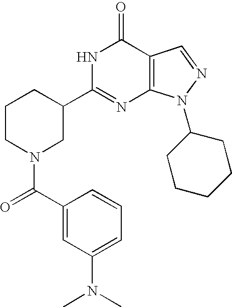 | 448.57 | 449 |
| 46 | 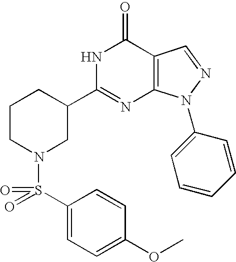 | 465.53 | 466 |
| 47 | 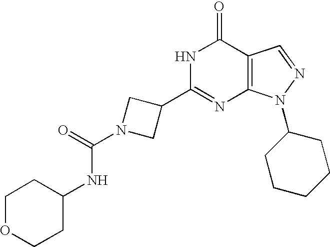 | 400.48 | 401 |
| 48 | 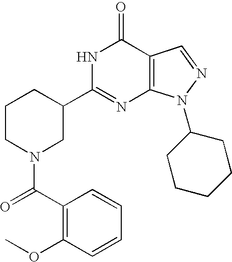 | 435.53 | 436 |
| 49 | 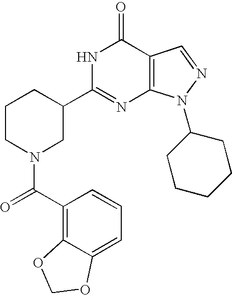 | 449.51 | 450 |
| 50 | 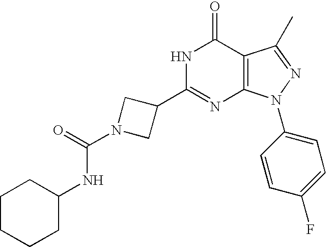 | 424.48 | 425 |
| 51 | 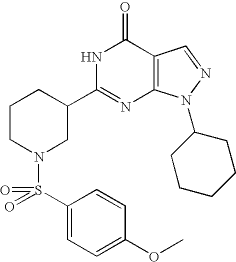 | 471.58 | 472 |
| 52 | 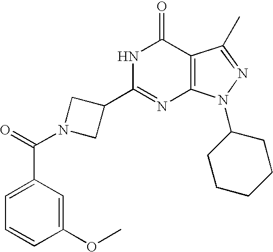 | 421.50 | 422 |
| 53 | 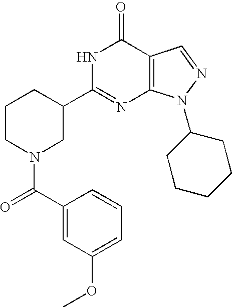 | 435.53 | 436 |
| 54 | 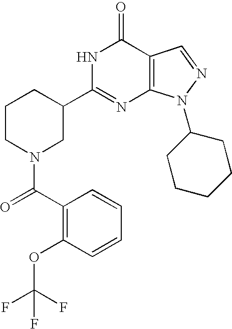 | 489.50 | 490 |
| 55 | 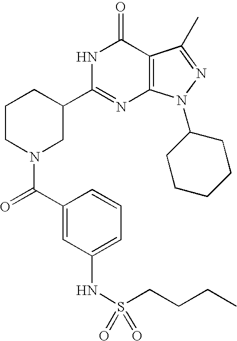 | 554.72 | 555 |
| 56 | 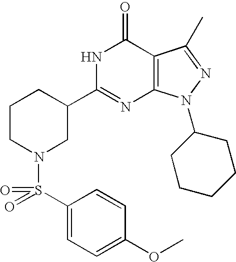 | 485.61 | 486 |
| 57 | 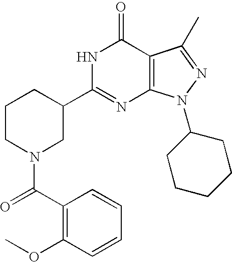 | 449.56 | 450 |
| 58 | 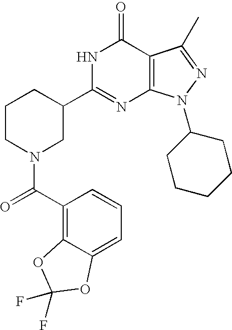 | 499.52 | 500 |
| 59 | 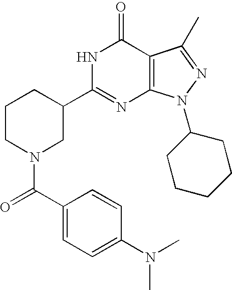 | 462.60 | 463 |
| 60 | 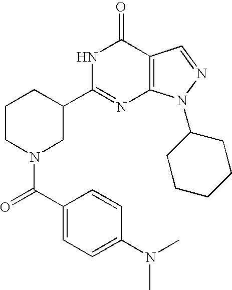 | 448.57 | 449 |
| 61 | 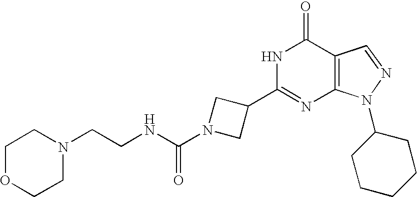 | 429.53 | 430 |
| 62 | 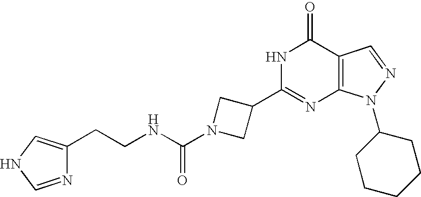 | 410.48 | 411 |
| 63 | 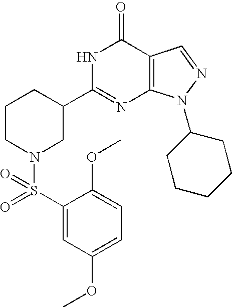 | 501.61 | 502 |
| 64 | 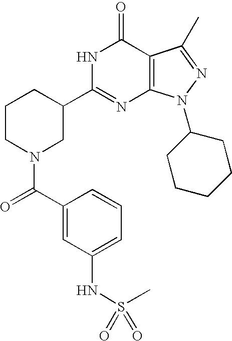 | 512.64 | 513 |
| 65 | 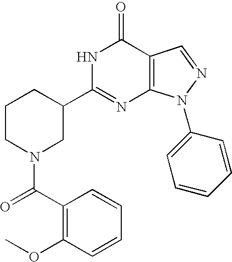 | 429.48 | 430 |
| 66 | 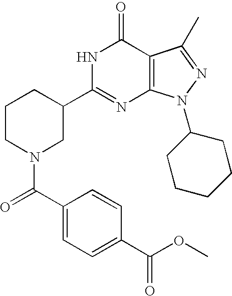 | 477.57 | 478 |
| 67 | 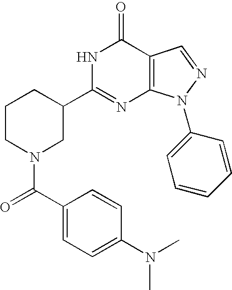 | 442.52 | 443 |
| 68 | 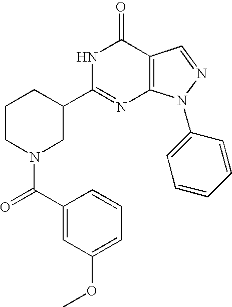 | 429.48 | 430 |
| 69 | 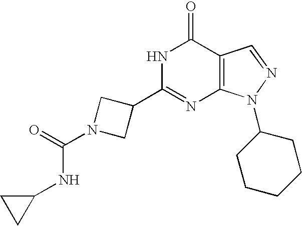 | 356.43 | 357 |
| 70 | 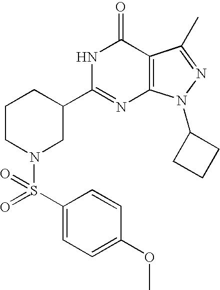 | 457.56 | 458 |
| 71 | 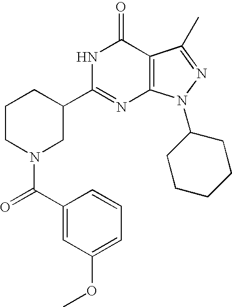 | 449.56 | 450 |
| 72 | 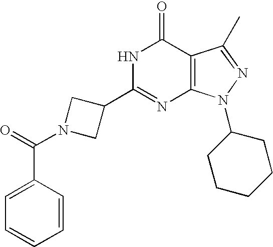 | 391.48 | 392 |
| 73 | 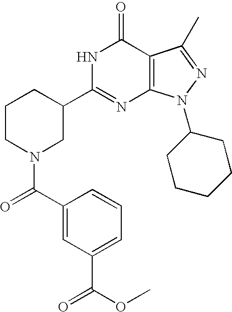 | 477.57 | 478 |
| 74 | 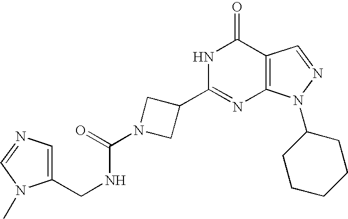 | 410.48 | 411 |
| 75 | 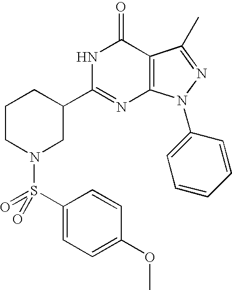 | 479.56 | 480 |
| 76 | 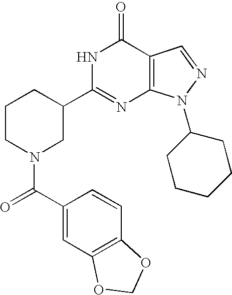 | 449.51 | 450 |
| 77 | 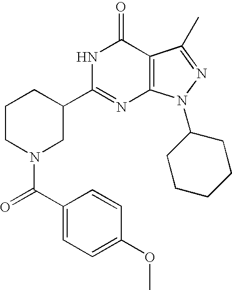 | 449.56 | 450 |
| 78 | 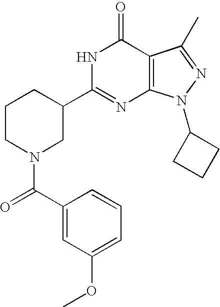 | 421.50 | 422 |
| 79 | 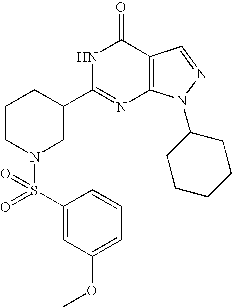 | 471.58 | 472 |
| 80 | 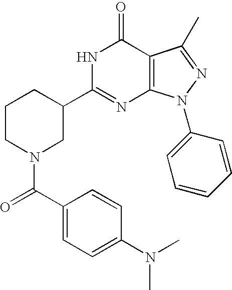 | 456.55 | 457 |
| 81 | 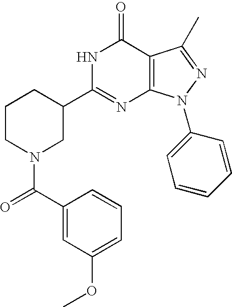 | 443.51 | 444 |
| 82 | 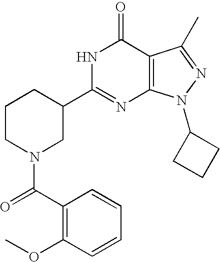 | 421.50 | 422 |
| 83 | 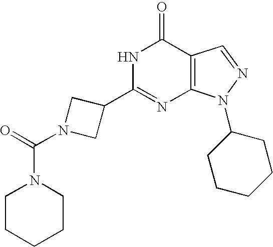 | 384.49 | 385 |
| 84 | 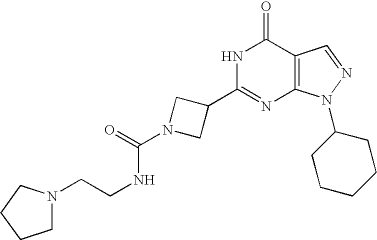 | 413.53 | 414.5 |
| 85 | 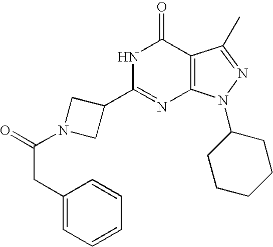 | 405.50 | 406 |
| 86 | 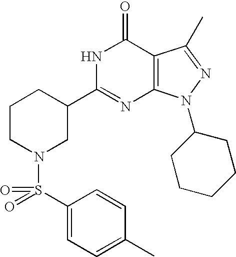 | 469.61 | 470 |
| 87 | 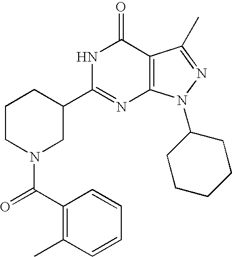 | 433.56 | 434 |
| 88 | 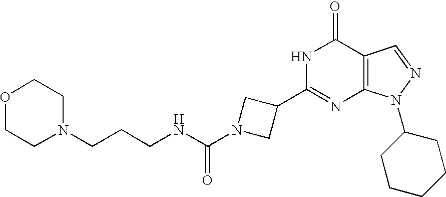 | 443.55 | 444 |
| 89 | 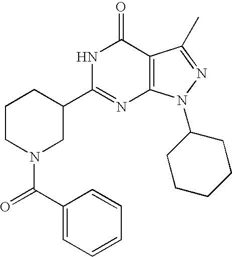 | 419.53 | 420 |
| 90 | 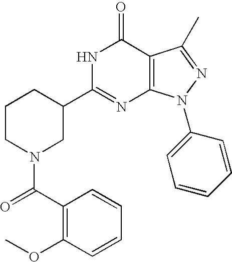 | 443.51 | 444 |
| 91 | 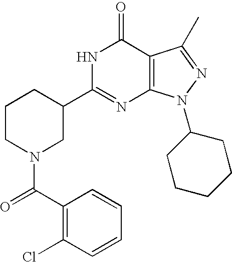 | 453.98 | 454, 456 |
| 92 | 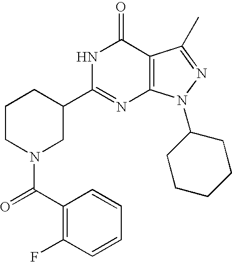 | 437.52 | 438 |
| 93 | 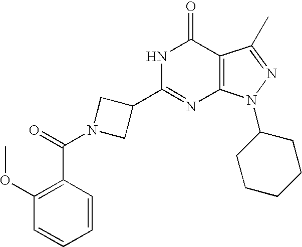 | 421.50 | 422 |
| 94 | 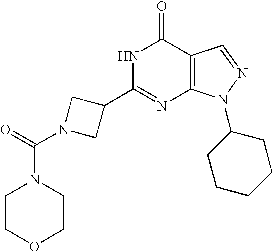 | 386.46 | 387 |
| 95 | 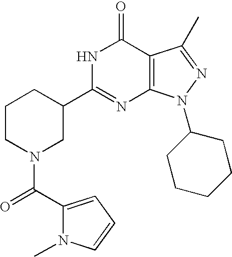 | 422.53 | 423 |
| 96 | 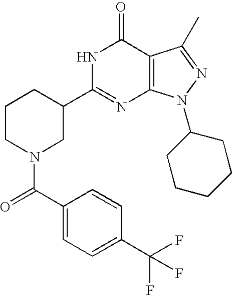 | 487.53 | 488 |
| 97 | 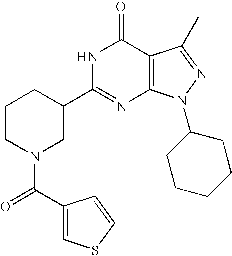 | 425.56 | 426 |
| 98 | 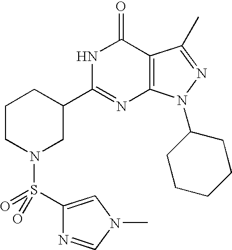 | 459.57 | 460 |
| 99 | 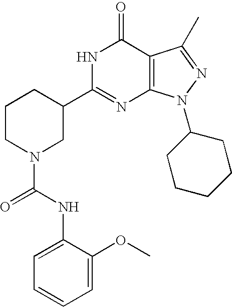 | 464.57 | 426 |
| 100 | 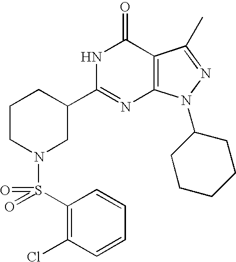 | 490.03 | 490, 492 |
| 101 | 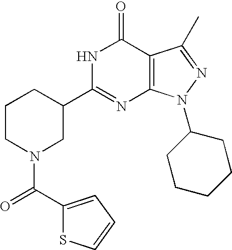 | 425.56 | 426 |
| 102 | 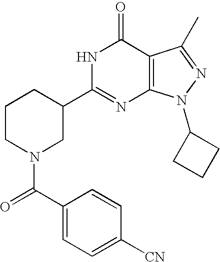 | 416.49 | 417 |
| 103 | 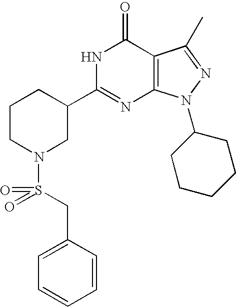 | 469.61 | 470 |
| 104 | 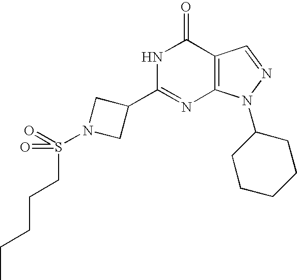 | 407.54 | 408 |
| 105 | 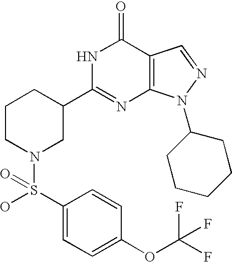 | 525.55 | 526 |
| 106 | 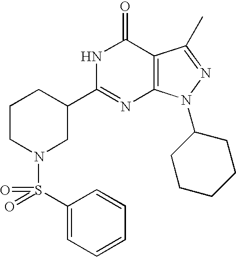 | 455.58 | 456 |
| 107 | 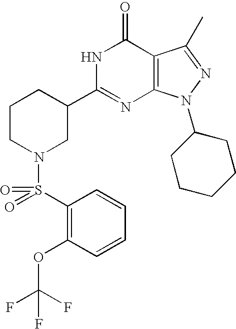 | 539.58 | 540 |
| 108 | 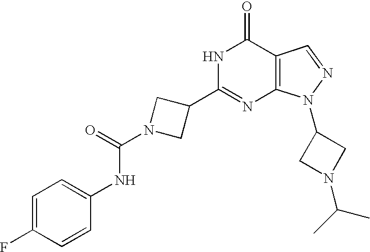 | 425.47 | 426 |
| 109 | 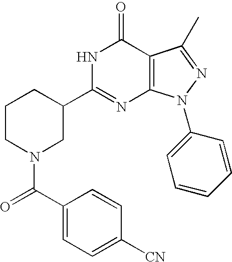 | 438.49 | 439 |
| 110 | 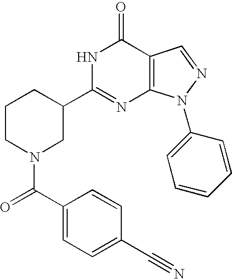 | 424.47 | 425 |
| 111 | 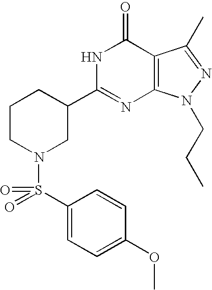 | 445.54 | 446 |
| 112 | 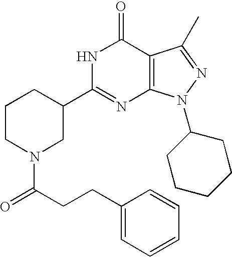 | 447.59 | 448 |
| 113 | 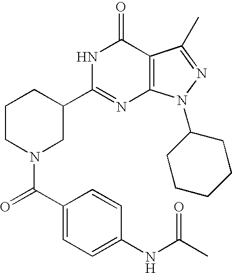 | 476.58 | 477 |
| 114 | 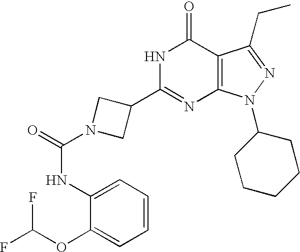 | 486.53 | 487.5 |

### Example 115

### Benzyl 3-(1-cyclohexyl-3-methyl-4-oxo-4,5-dihydro-1H-pyrazolo[3,4-d]pyrimidin-6-yl)azetidine-1-carboxylate

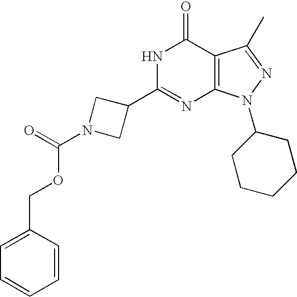

Benzyl chloroformate (37 μL, 0.28 mmol) was added to a suspension of Description 7 (60 mg, 0.19 mmol) and morpholinomethyl PS resin (4.2 mmol/g, 180 mg, 0.74 mmol) in DCM (3 mL) and the reaction shaken. After 16 h additional benzyl chloroformate (37 μL, 0.28 mmol) was added and the reaction shaken for a further 4 h. Methyl isocyanate PS resin (100 mg) and tris-(2-aminoethyl)-amine PS resin (100 mg) were added and the reaction shaken for 16 h. The reaction was filtered and the resins rinsed with DCM/MeOH. The filtrate was evaporated and the crude mixture purified by flash column chromatography eluting with ethyl acetate to give the title compound (14 mg, 18%). ¹H NMR (400 MHz, CDCl₃): δ 11.60 (s), 7.34 (5H, s), 5.14 (2H, s) 4.58 (1H, m), 4.41 (4H, m), 3.86 (1H, pent, J=6 Hz), 2.58 (3H, s), 2.04-1.99 (6H, m), 1.90 (1H, m), 1.47 (1H, m), 1.33 (2H, m). MS (MH⁺, m/z) 422.

### Example 116

### 4-(4-Isopropylpiperazin-1-yl)phenyl 3-(1-tert-butyl-4-oxo-4,5-dihydro-1H-pyrazolo [3,4-d]pyrimidin-6-yl)azetidine-1-carboxylate

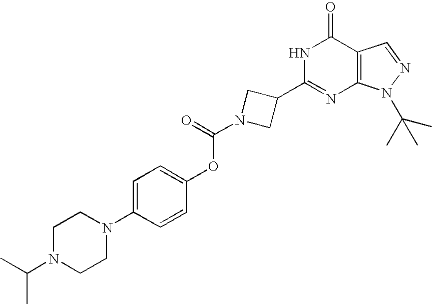

1-[4-(4-Hydroxy-phenyl)-piperazin-1-yl]-ethanone (Description 18, 66 mg, 0.30 mmol) was added to a solution of disuccinimidyl carbonate (0.3 M in MeCN, 1.25 mL, 0.375 mmol) and triethylamine (72 μL, 0.50 mmol) in MeCN (3 mL). The mixture was stirred for 4 h then Description 6 (71 mg, 0.25 mmol) was added. The mixture was stirred for 16 h then the solvent was removed in vacuo. The residue was dissolved in DCM (5 mL) then washed with Na₂CO₃ solution (1M, 5 mL). The organics were removed in vacuo and the residue purified by gradient column chromatography eluting with 4-6% MeOH in DCM to give the title compound (34 mg, 27%). ¹H NMR (400 MHz, d6-DMSO): δ 12.25 (1H, br), 8.01 (1H, s), 7.00-6.93 (4H, m), 4.52-4.35 (4H, m), 3.97 (1H, m), 3.12 (4H, m), 2.72 (1H, m), 2.60 (4H, m), 1.77 (9H, s), 1.04 (6H, d, J=8 Hz). MS (MH⁺, m/z) 494 (parent).

Examples 117-124 in Table 3 were made according to the procedure of Example 115 using the appropriate chloroformate or according to the procedure of Example 116 using the appropriate alcohol.

TABLE 3

| | | | MS |
|---|---|---|---|
| EXAMPLE | | | (MH⁺, |
| # | STRUCTURE | MW | m/z) |
| 117 | 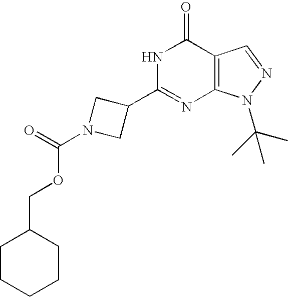 | 387.49 | 388 |
| 118 | 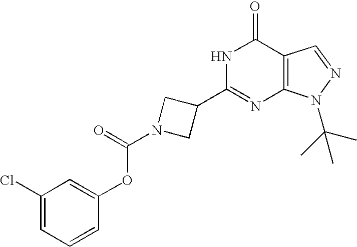 | 401.86 | 402 |
| 119 | 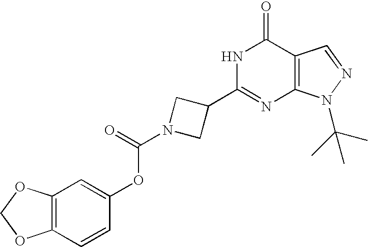 | 411.42 | 412 |
| 120 | 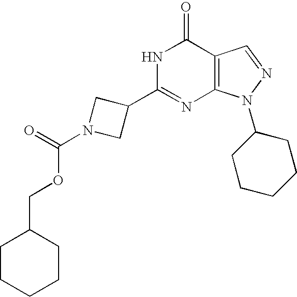 | 413.52 | 414 |
| 121 | 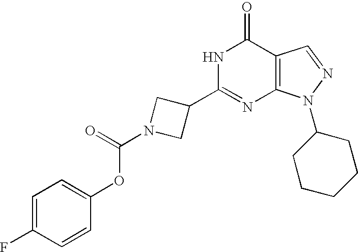 | 411.44 | 412 |
| 122 | 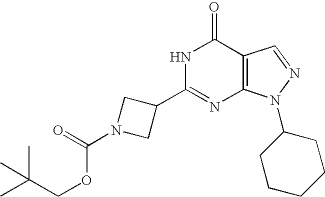 | 387.49 | 388.5 |
| 123 | 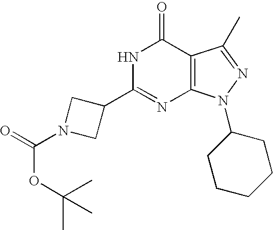 | 387.49 | 388 |
| 124 | 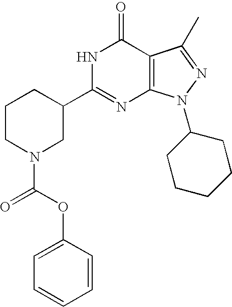 | 435.53 | 436 |

### Example 125

### 1-Cyclohexyl-6-(1-(1H-benzo[d]imidazol-2-yl)azetidin-3-yl)-1H-pyrazolo[3,4-d]pyrimidin-4-(5H)-one

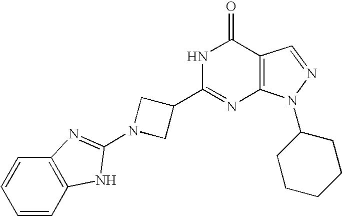

A sealed tube was charged with 6-azetidin-3-yl-1-cyclohexyl-1,5-dihydropyrazolo[3,4-d]pyrimidin-4-one hydrochloride (Description 4, 200 mg, 0.64 mmol), 2-chloro-1H-benzimidazole (111 mg, 0.73 mmol), potassium carbonate (265 mg, 1.92 mmol), 2-propanol (2 mL) and water (1 mL). The mixture was heated at 120° C. for 1 h under microwave irradiation. After return to room temperature, solvents were removed under reduced pressure. The residue was diluted in a mixture of MeOH (10 mL) and DCM (10 mL) and filtered. The filtrate was evaporated and the crude product was purified by gradient column chromatography on silica gel eluting with 3-10% MeOH in DCM to give the title compound as a white solid (50 mg, 20%). ¹H NMR (400 MHz, d₆-DMSO): δ 12.22 (1H, s), 11.49 (1H, s), 8.01 (1H, s), 7.32-7.18 (2H, m), 7.01-6.86 (2H, m), 4.62-4.51 (1H, m), 4.42-4.37 (4H, m), 4.18-4.03 (1H, m), 1.90-1.78 (6H, m), 1.71-1.64 (1H, m), 1.47-1.32 (2H, m), 1.28-1.17 (1H, m). MS (MH⁺, m/z) 390.

Examples 126-144 in Table 4 were made from the appropriate chloride, bromide or sulfonic acid according to the procedure of Example 125.

TABLE 4

| | | | MS |
|---|---|---|---|
| EXAMPLE | | | (MH⁺, |
| # | STRUCTURE | MW | m/z) |
| 126 | 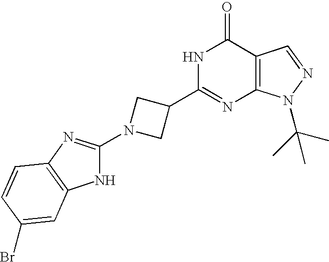 | 442.32 | 442, 444 |
| 127 | 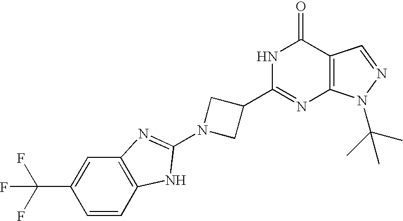 | 431.42 | 432 |
| 128 | 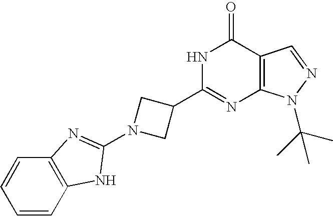 | 363.43 | 364 |
| 129 | 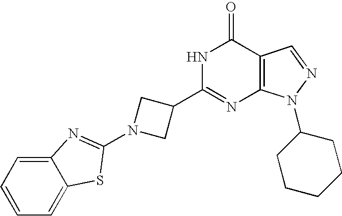 | 406.51 | 407 |
| 130 | 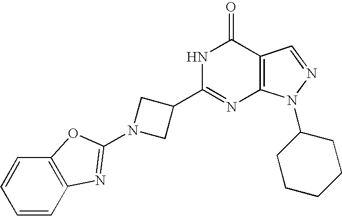 | 390.45 | 391 |
| 131 | 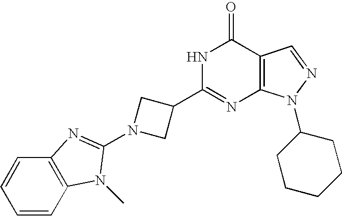 | 403.49 | 404 |
| 132 | 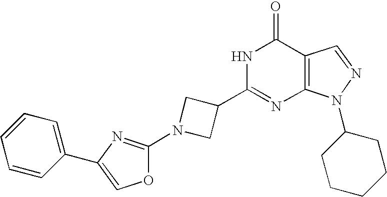 | 416.49 | 417 |
| 133 | 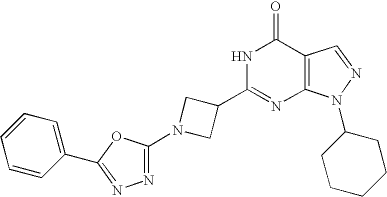 | 417.47 | 418 |
| 134 | 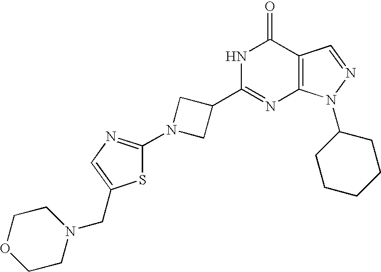 | 455.59 | 456 |
| 135 | 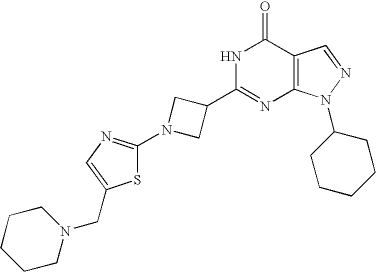 | 453.61 | 454 |
| 136 | 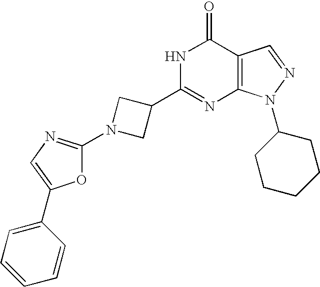 | 416.49 | 417 |
| 137 | 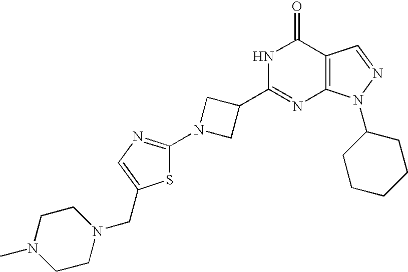 | 468.63 | 469 |
| 138 | 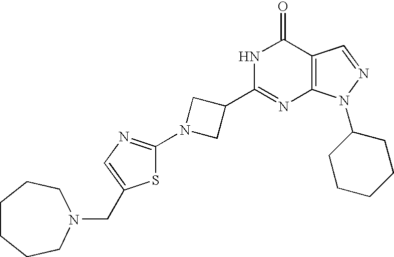 | 467.64 | 468 |
| 139 | 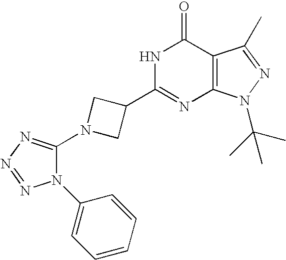 | 405.47 | 406 |
| 140 | 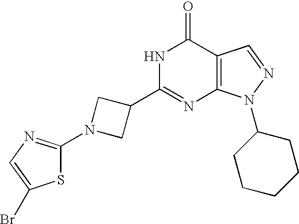 | 435.35 | 435, 437 |
| 141 | 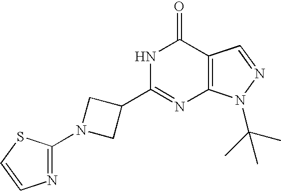 | 330.41 | 331 |
| 142 | 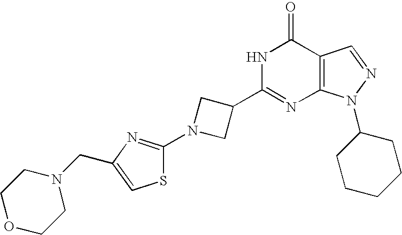 | 455.59 | 456 |
| 143 | 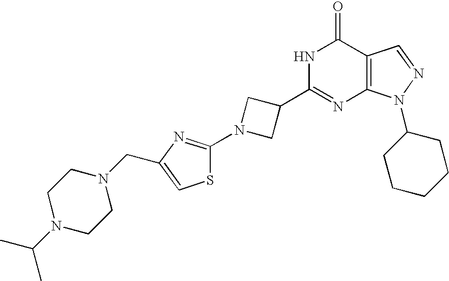 | 496.68 | 497 |
| 144 | 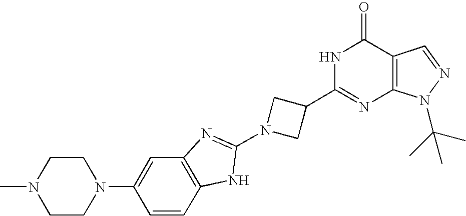 | 461.57 | 462 |

### Example 145

### 1-Cyclohexyl-6-(1-(4-(Morpholinosulfonyl)phenyl)azetidin-3-yl)-1H-pyrazolo[3,4-d]pyrimidin-4(5H)-one

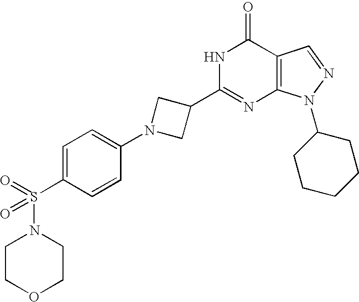

A mixture of Description 4 (86 mg, 0.278 mmol), 4-(4-fluoro-benzenesulfonyl)morpholine (Description 23, 85 mg, 0.349 mmol) and potassium carbonate (115 mg, 0.834 mmol) in DMSO (2 mL) was irradiated in the microwave at 120° C. for 45 min. The reaction was partitioned between water and EtOAc. The organic layer was washed with water, dried over MgSO₄ and evaporated to give crude product that was purified by gradient column chromatography, eluting with 2.5-5% MeOH in DCM to give the title compound as a white solid (82 mg, 61%). ¹H NMR (400 MHz, CDCl₃): δ 11.40 (1H, s), 8.06 (1H, s), 7.63 (2H, d, J=8 Hz), 6.56 (2H, d, J=8 Hz), 4.65 (1H, m), 4.43 (2H, m), 4.35 (2H, m), 4.08 (1H, m), 3.75 (4H, t, J=6 Hz), 2.98 (4H, t, J=4 Hz), 1.98 (6H, m), 1.76 (1H, m), 1.45 (2H, m), 1.31 (1H, m). (MH⁺, m/z) 499.

Examples 146-154 in Table 5 have been prepared according to the procedure of Example 145.

TABLE 5

| | | | MS |
|---|---|---|---|
| EXAMPLE | | | (MH⁺, |
| # | STRUCTURE | MW | m/z) |
| 146 | 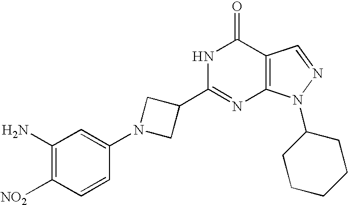 | 409.45 | 410 |
| 147 | 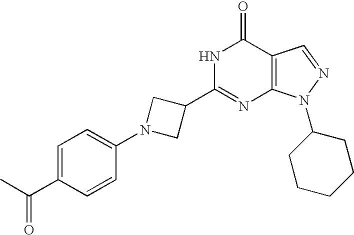 | 391.48 | 392 |
| 148 | 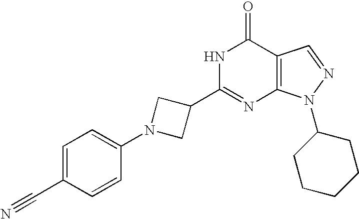 | 374.45 | 375 |
| 149 | 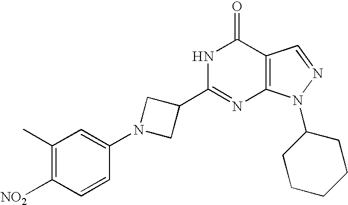 | 408.46 | 409 |
| 150 | 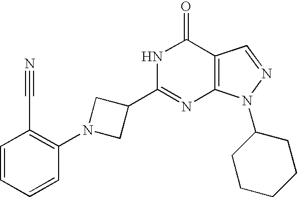 | 374.45 | 375 |
| 151 | 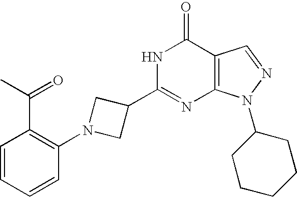 | 391.48 | 392 |
| 152 | 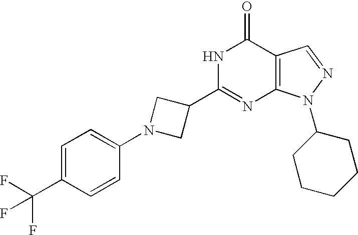 | 417.44 | 418 |
| 153 | 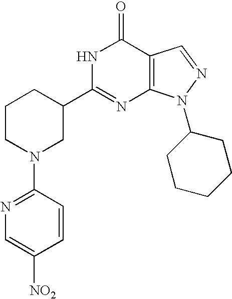 | 423.48 | 424 |
| 154 | 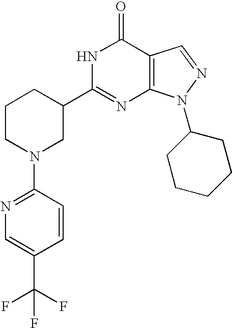 | 446.48 | 447 |

### Example 155

### 1-Cyclohexyl-6-[1-(4-morpholin-4-ylmethyl-phenyl)-azetidin-3-yl]-1,5-dihydro-pyrazolo[3,4-d]pyrimidin-4-one

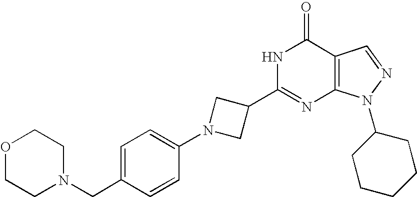

### Step 1: 4-[3-(1-Cyclohexyl-4-oxo-4,5-dihydro-1H-pyrazolo[3,4-d]pyrimidin-6-yl)-azetidin-1-yl]-benzaldehyde

Prepared from Description 4 and 4-fluoro benzaldehyde according to the procedure of Example 145.

### Step 2: 1-Cyclohexyl-6-[1-(4-morpholin-4-ylmethyl-phenyl)-azetidin-3-yl]-1,5-dihydropyrazolo[3,4-d]pyrimidin-4-one

The product of Step 1 was treated with acetone according to the procedure of Description 17 to give the title compound. ¹H NMR (400 MHz, d6-DMSO): δ 12.15 (1H, s), 8.04 (1H, s), 7.14 (2H, d, J=8 Hz), 6.49 (2H, d, J=8 Hz), 4.14 (4H, m), 4.05 (1H, m), 3.58 (4H, m), 3.36 (2H, s), 2.33 (4H, m), 1.88 (6H, m), 1.69 (1H, m), 1.45 (2H, m), 1.24 (1H, m). ([M-H]⁻, m/z) 447.

### Example 156

### 1-Cyclohexyl-6-(1-(2-(morpholinomethyl)phenyl)azetidin-3-yl)-1H-pyrazolo[3,4-d]pyrimidin-4(5H)-one

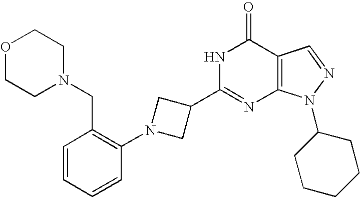

Prepared using a procedure analogous to that described in Example 155.

### Example 157

### 6-(1-(4-tert-Butylthiazol-2-yl)azetidin-3-yl)-1-cyclohexyl-1H-pyrazolo[3,4-d]pyrimidin-4-(5H)-one

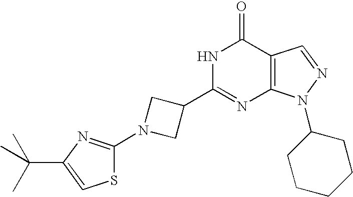

### Step 1: N-[3-(1-Cyclohexyl-4-oxo-4,5-dihydro-1H-pyrazolo[3,4-d]pyrimidin-6-yl)azetidine-1-carbothioyl]benzamide

Benzoylisothiocyanate (111 μL, 0.77 mmol) was added to a solution of Description 4 (200 mg, 0.64 mmol) and triethylamine (90 μL, 0.64 mmol) in DCM (6 mL). The reaction was stirred at room temperature for 18 h, then diluted with DCM (10 mL) and washed with water (3×10 mL). The organic phase was collected, dried (MgSO₄) and evaporated. The residue was purified by column chromatography on silica gel eluting with 5% dichloromethane in methanol to give N-[3-(1-cyclohexyl-4-oxo-4,5-dihydro-1H-pyrazolo[3,4-d]pyrimidin-6-yl)azetidine-1-carbothioyl]benzamide as an orange solid (112 mg, 39%). ¹H NMR (400 MHz, CDCl₃): δ 8.75 (1H, s), 8.02 (1H, s), 7.81-7.69 (2H, m), 7.62-7.45 (3H, m), 4.86-4.50 (5H, m), 4.09-4.06 (1H, m), 2.08-1.92 (6H, m), 1.80-1.76 (1H, m), 1.54-1.21 (3H, m). MS (MH⁺, m/z) 437.

### Step 2: 3-(1-Cyclohexyl-4-oxo-4,5-dihydro-1H-pyrazolo[3,4-d]pyrimidin-6-yl)-azetidine-1-carbothioic Acid Amide

A solution of N-[3-(1-cyclohexyl-4-oxo-4,5-dihydro-1H-pyrazolo[3,4-d]pyrimidin-6-yl)azetidine-1-carbothioyl]benzamide (220 mg, 0.50 mmol) in hydrazine monohydrate (3 mL) was stirred at room temperature for 2 h. Water was added (10 mL) and the reaction was extracted with DCM (3×20 mL). The organic phases were combined, dried (MgSO₄) and evaporated. The residue was purified by flash chromatography on silica gel eluting with 5% DCM in MeOH to give 3-(1-cyclohexyl-4-oxo-4,5-dihydro-1H-pyrazolo[3,4-d]pyrimidin-6-yl)-azetidine-1-carbothioic acid amide as a white solid (50 mg, 30%). ¹H NMR (400 MHz, CDCl₃): δ 8.07 (1H, s), 5.97 (2H, s), 4.68-4.66 (1H, m), 4.64-4.62 (4H, m), 3.98-3.94 (1H, m), 2.03-1.91 (6H, m), 1.78-1.75 (1H, m), 1.51-1.43 (2H, m), 1.36-1.25 (1H, m). MS (MH⁺, m/z) 333.

### Step 3: 6-(1-(4-tert-Butylthiazol-2-yl)azetidin-3-yl)-1-cyclohexyl-1H-pyrazolo[3,4-d]pyrimidin-4(5H)-one

A solution of 3-(1-cyclohexyl-4-oxo-4,5-dihydro-1H-pyrazolo[3,4-d]pyrimidin-6-yl)azetidine-1-carbothioic acid amide (50 mg, 0.15 mmol) and 1-chloropinacolone (20 μL, 0.15 mmol) in EtOH (3 mL) was heated at 90° C. for 5 h. After return to room temperature, EtOH was removed under reduced pressure and the residue was purified by flash chromatography on silica gel eluting with 10% DCM in MeOH to give the title compound as a white solid. ¹H NMR (400 MHz, CDCl₃): δ 11.92 (1H, s), 8.06 (1H, s), 6.22 (1H, s), 4.67-4.65 (1H, m), 4.49-4.41 (4H, m), 4.11-4.08 (1H, m), 2.03-1.91 (6H, m), 1.78-1.75 (1H, m), 1.53-1.44 (2H, m), 1.32-1.28 (10H, m). MS (MH⁺, m/z) 413.

### Example 158

### 1-Cyclohexyl-6-(1-(5-phenyl-4H-1,2,4-triazol-3-yl)azetidin-3-yl)-1H-pyrazolo[3,4-d]pyrimidin-4-(5H)-one

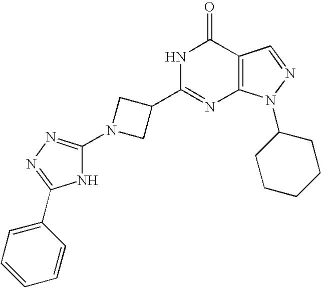

A solution of N-[3-(1-cyclohexyl-4-oxo-4,5-dihydro-1H-pyrazolo[3,4-d]pyrimidin-6-yl)azetidine-1-carbothioyl]benzamide (Example 157, step 1, 112 mg, 0.25 mmol) and hydrazine monohydrate (61 μL, 1.28 mmol) in chloroform was heated at reflux for 4 h. The reaction was then evaporated to dryness and the residue was purified by flash chromatography on silica gel eluting with 5% DCM in MeOH to give a mixture of targeted compound and a by-product. The final compound was then isolated by preparative HPLC (6 mg, 6%). ¹H NMR (400 MHz, d₆-DMSO): δ 13.01 (1H, s), 12.21 (1H, s), 8.04 (1H, s), 7.97-7.94 (2H, m), 7.49-7.42 (3H, m), 4.60-4.57 (1H, m), 4.34-4.32 (4H, m), 4.11-4.07 (1H, m), 1.90-1.82 (6H, m), 1.70-1.66 (1H, m), 1.45-1.39 (2H, m), 1.27-1.22 (1H, m). MS (MH⁺, m/z) 417.

### Example 159

### 1-Cyclohexyl-6-(1-(4-phenylthiazol-2-yl)azetidin-3-yl)-1H-pyrazolo[3,4-d]pyrimidin-4-(5H)-one

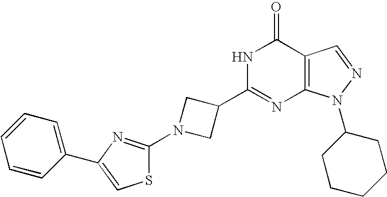

### Step 1: 1-Cyclohexyl-6-(1-(4-bromothiazol-2-yl)azetidin-3-yl)-1H-pyrazolo[3,4-d]pyrimidin-4-(5H)-one

1-Cyclohexyl-6-(1-(4-bromothiazol-2-yl)azetidin-3-yl)-1H-pyrazolo[3,4-d]pyrimidin-4-(5H)-one was prepared from Description 4 and 2,4-dibromothiazole according to the procedure of Example 125. ¹H NMR (400 MHz, CDCl₃): δ 12.87 (1H, s), 8.06 (1H, s), 6.50 (1H, s), 4.69-4.63 (1H, m), 4.56-4.52 (4H, m), 4.23-4.18 (1H, m), 2.07-1.92 (6H, m), 1.79-1.76 (1H, m), 1.54-1.45 (2H, m), 1.33-1.26 (1H, m). MS (MH⁺, m/z) 435/437.

### Step 2: 1-Cyclohexyl-6-(1-(4-phenylthiazol-2-yl)azetidin-3-yl)-1H-pyrazolo[3,4-d]pyrimidin-4-(5H)-one

To a solution of 1-cyclohexyl-6-(1-(4-bromothiazol-2-yl)azetidin-3-yl)-1H-pyrazolo[3,4-d]pyrimidin-4-(5H)-one (150 mg, 0.34 mmol) in toluene (10 mL) was successively added tetrakis (triphenylphosphine)palladium(0) (40 mg, 0.034 mmol), phenylboronic acid (48 mg, 0.39 mmol) and 1.5 M sodium carbonate solution (680 μL, 1.02 mmol). The resulting mixture was heated at 100° C. for 4 h. Toluene was removed under reduced pressure and the residue was partitioned between DCM (150 mL) and water (50 mL). The organic phase was separated, washed with brine (50 mL), dried (MgSO₄) and evaporated. The residue was purified by flash chromatography on silica gel eluting with 5% DCM in MeOH to give the title compound (35 mg, 23%). ¹H NMR (400 MHz, CDCl₃): δ 12.46 (1H, s), 8.07 (1H, s), 7.85-7.82 (2H, m), 7.47-7.44 (2H, m), 7.38-7.35 (1H, m), 6.83 (1H, s), 4.69-4.67 (1H, m), 4.64-4.52 (4H, m), 4.22-4.17 (1H, m), 2.05-1.93 (6H, m), 1.76-1.73 (1H, m), 1.50-1.46 (2H, m), 1.19-1.14 (1H, m). MS (MH⁺, m/z) 433.

### Example 160

### 1-Cyclohexyl-6-(1-(5-phenylthiazol-2-yl)azetidin-3-yl)-1H-pyrazolo[3,4-d]pyrimidin-4-(5H)-one

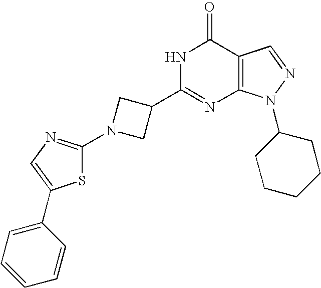

1-Cyclohexyl-6-(1-(5-phenylthiazol-2-yl)azetidin-3-yl)-1H-pyrazolo[3,4-d]pyrimidin-4-(51H)-one was prepared from Description 4 and 2,5-dibromothiazole according to the procedure of Example 159, Step 2. ¹H NMR (400 MHz, CDCl₃): δ 12.02 (1H, s), 8.07 (1H, s), 7.45-7.40 (3H, m), 7.37-7.35 (2H, m), 7.26-7.22 (1H, m), 4.70-4.67 (1H, m), 4.62-4.56 (4H, m), 4.21-4.14 (1H, m), 2.06-1.93 (6H, m), 1.76-1.73 (1H, m), 1.49-1.46 (2H, m), 1.19-1.13 (1H, m). MS (MH⁺, m/z) 433.

### Example 161

### 1-Cyclohexyl-6-(1-(4-(4-(4-isopropylpiperazin-1-yl)phenyl)thiazol-2-yl)azetidin-3-yl)-1H-pyrazolo[3,4-d]pyrimidin-4-(5H)-one

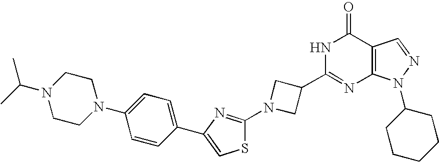

1-Cyclohexyl-6-(1-(4-(4-(4-isopropylpiperazin-1-yl)phenyl)thiazol-2-yl)azetidin-3-yl)-1H-pyrazolo[3,4-d]pyrimidin-4-(5H)-one was prepared from Example 159, Step 1 and 4-(4-isopropylpiperazin-1-yl)phenyl boronic acid according to the procedure of Example 159, Step 2. ¹H NMR (400 MHz, CDCl₃): δ 12.21 (1H, s), 8.06 (1H, s), 7.74-7.72 (2H, m), 6.93-6.90 (2H, m), 6.68 (1H, s), 4.67-4.65 (1H, m), 4.54-4.52 (4H, m), 4.20-4.17 (1H, m), 3.48-3.30 (4H, m), 3.14-3.09 (1H, m), 2.99-2.73 (4H, m), 2.01-1.89 (6H, m), 1.76-1.73 (1H, m), 1.49-1.46 (2H, m), 1.28-1.07 (7H, m). MS (MH⁺, m/z) 559.

### Example 162

### 1-Cyclohexyl-6-(1-(5-morpholin-4-ylmethyl-4-phenylthiazol-2-yl)azetidin-3-yl)-1H-pyrazolo[3,4-d]pyrimidin-4-(5H)-one

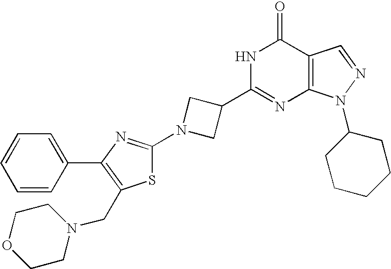

Phosphorus(III) oxychloride (8 μL, 0.08 mmol) was added under nitrogen to DMF (2 mL) at 0° C. The mixture was stirred at 0° C. for 30 min then a solution of 1-cyclohexyl-6-(1-(4-phenylthiazol-2-yl)azetidin-3-yl)-1H-pyrazolo[3,4-d]pyrimidin-4-(5H)-one (Example 159, 24 mg, 0.05 mmol) in DMF (1 mL) was added. The reaction was stirred at 0° C. for 30 min then at room temperature for 4 h. Aqueous sodium hydroxide (6N, 5 mL) was then added and the reaction was extracted with EtOAc (2×10 mL). Organic phases were combined and washed with water, dried (MgSO₄) and evaporated. The residue was dissolved in MeOH (4 mL) and acetic acid (3 μL, 0.05 mmol) then morpholine (6 μL, 0.07 mmol) was added. The resulting mixture was stirred at room temperature for 30 min. Sodium cyanoborohydride (0.10 mmol, 6.3 mg) was added and the mixture was stirred overnight at room temperature. The reaction was evaporated to dryness and the residue taken up in MeOH (1.5 mL) and filtered. The filtrate was purified by preparative HPLC to provide the title compound (3 mg, 11%). ¹H NMR (400 MHz, d₆-DMSO): δ 12.21 (1H, s), 7.98 (1H, s), 7.61-7.59 (2H, m), 7.47-7.43 (2H, m), 7.39-7.35 (1H, m), 4.60-4.57 (1H, m), 4.36-4.34 (4H, m), 4.12-4.09 (1H, m), 3.65 (2H, s), 3.61-3.59 (4H, m), 2.44-2.36 (4H, m), 1.94-1.83 (6H, m), 1.71-1.70 (1H, m), 1.50-1.44 (2H, m), 1.25-1.22 (1H, m). MS (MH⁻, m/z) 530.

### Example 163

### 1-tert-Butyl-6-(1-pyridin-2-yl-azetidin-3-yl)-1,5-dihydro-pyrazolo[3,4-d]pyrimidin-4-one

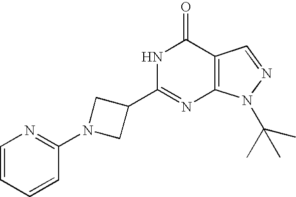

6-Azetidin-3-yl-1-tert-butyl-1,5-dihydro-pyrazolo[3,4-d]pyrimidin-4-one hydrochloride (Description 6, 40 mg. 0.14 mmol), 2-bromopyridine (12 μL, 0.12 mmol), Pd₂(dba)₃ (1 mol %, 1.2 mg) and 2-(dicyclohexylphosphino)biphenyl (2.5 mol %, 1 mg) were mixed in a screw-top vial under N₂. LiHMDS (1M in THF, 0.38 mL, 0.38 mmol) was added, the flask sealed and the reaction heated to 80° C. for 16 h. The mixture was cooled and HCl (1M, 0.5 mL) was added. The mixture was stirred for 5 min. Na₂CO₃ (1M, 1 mL) was then added followed by a further 5 min stirring. DCM (4 mL) was added and the organic layer was collected, dried over MgSO₄ and concentrated in vacuo to give the crude product. This was purified by gradient column chromatography on silica gel, using 3-9% MeOH in DCM, to give the title compound as a white solid (30 mg, 77%). ¹H NMR (400 MHz, d6-DMSO): δ 12.20 (1H, br s), 8.13°-8.10 (1H, m), 7.98 (1H, s), 7.60-7.53 (1H, m), 6.71-6.68 (1H, m), 6.52-6.48 (1H, m), 4.31-4.21 (4H, m), 4.10-4.00 (1H, m), 1.69 (9H, s). MS (MH⁺, m/z) 325.

### Example 164

### 1-tert-Butyl-6-[1-(4-morpholin-4-ylmethyl-phenyl)-azetidin-3-yl]-1,5-dihydro-pyrazolo[3,4-d]pyrimidin-4-one

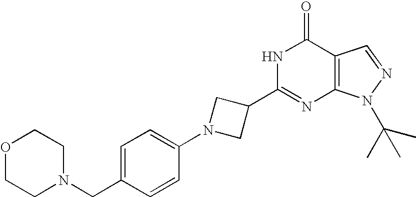

Prepared from Description 6 and 4-(4-bromobenzyl)-morpholine (Description 22) according to the procedure of Example 163. The crude product was purified by gradient column chromatography, using 2-5% MeOH in DCM, to give the product as a white solid (79 mg, 69%). ¹H NMR (400 MHz, d6-DMSO): δ 12.18 (1H, br s), 7.98 (1H, s), 7.14 (2H, d, J=8), 6.49 (2H, d, J=8), 4.20-4.15 (2H, m), 4.13-4.03 (3H, m), 3.61-3.55 (4H, m), 2.38-2.30 (4H, m), 1.70 (9H, m). MS (MH⁺, m/z) 421.

Examples 165-169 in Table 6 were made from commercially available aryl bromides according to the procedure of Example 163.

TABLE 6

| | | | MS |
|---|---|---|---|
| EXAMPLE | | | (MH⁺, |
| # | STRUCTURE | MW | m/z) |
| 165 | 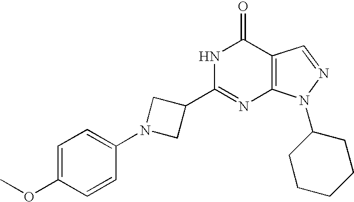 | 379.47 | 378 [M − H⁺]⁻ |
| 166 | 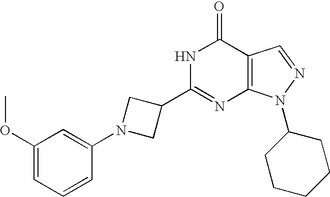 | 379.47 | 378 [M − H⁺]⁻ |
| 167 | 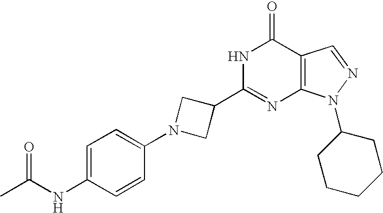 | 406.49 | 407 |
| 168 | 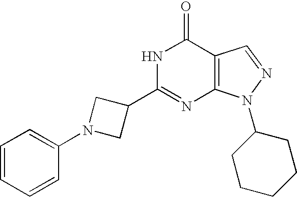 | 349.44 | 348 [M − H⁺]⁻ |
| 169 | 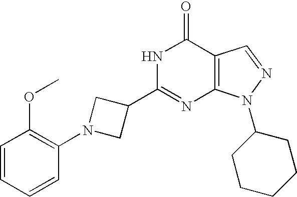 | 379.47 | 380 |

### Example 170

### 3-(1-Cyclohexyl-3-methyl-4-oxo-4,5-dihydro-1H-pyrazolo[3,4-d]pyrimidin-6-yl)-N-(4-fluorobenzyl)-N-methylazetidine-1-carboxamide

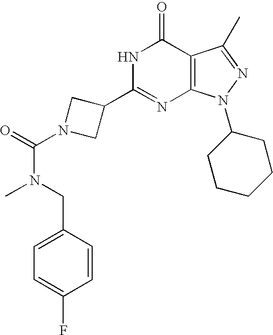

### Step 1: 3-[(4-Fluorobenzyl)-methyl-carbamoyl]-1-methyl-3H-imidazol-1-ium iodide

A solution of N-methyl-4-fluoro-benzylamine (420 mg, 0.40 mL, 3.0 mmol) and carbonyl diimidazole (0.54 g, 3.3 mmol) in THF (7 mL) was refluxed for 3 days. The reaction mixture was evaporated and the residue taken up in DCM and washed twice with water. The organic layer was dried over MgSO₄ and evaporated to give an oil (0.50 g). To this oil was added MeCN (5 mL) followed by methyl iodide (1.22 g, 0.53 mL, 8.6 mmol), and the resulting solution stirred at room temperature for 18 h. The reaction mixture was evaporated to give a viscous yellow oil, which was used without further purification (0.85 g, 76%). ¹H NMR (400 MHz, CDCl₃): δ 10.59 (1H, br s), 7.63 (1H, br s), 7.36 (3H, m), 7.07 (3H, m), 4.74 (2H, s), 4.27 (3H, s), 3.31 (3H, s).

### Step 2: 3-(1-Cyclohexyl-3-methyl-4-oxo-4,5-dihydro-1H-pyrazolo[3,4-d]pyrimidin-6-yl)-N-(4-fluorobenzyl)-N-methylazetidine-1-carboxamide

A suspension of Description 7 (66 mg, 0.205 mmol) and the product of Step 1 (100 mg, 0.267 mmol) was treated according to the procedure in Example 1 to give the title compound. ¹H NMR (400 MHz, CDCl₃): δ 10.48 (1H, br s), 7.25 (2H, t, J=8 Hz), 7.04 (2H, t, J=8 Hz), 4.45 (1H, m), 4.37 (2H, s), 4.33 (4H, m), 3.78 (1H, m), 2.81 (3H, s), 2.58 (3H, s), 1.95 (6H, m), 1.73 (1H, m), 1.45 (2H, m), 1.31 (1H, m). (MH⁺, m/z) 453.

### Example 171

### 1-tert-Butyl-6-(1-((1-methyl-1H-benzo[d]imidazol-2-yl)methyl)azetidin-3-yl)-1H-pyrazolo[3,4-d]pyrimidin-4(5H)-one

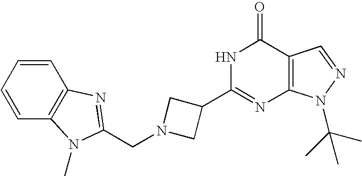

A solution of Description 6 (80 mg, 0.283 mmol), 1-methyl-2-carbonyl-benzimidazole (54 mg, 0.339 mmol) and acetic acid (19 mg, 18 mL, 0.311 mmol) in MeOH (2 mL) was stirred at room temperature. After 30 min sodium cyanoborohydride (36 mg, 0.566 mmol) was added and the reaction stirred at room temperature for 16 h. The reaction mixture was evaporated and partitioned between saturated sodium bicarbonate solution and DCM. The organic layer was dried over MgSO₄ and evaporated. The crude product was purified by column chromatography using EtOAc/DCM/MeOH (10:9:1) to elute impurities and then DCM/MeOH (9:1) to elute the product (86 mg, 78%). ¹H NMR (400 MHz, CDCl₃): δ 11.91 (1H, br s), 8.02 (1H, s), 7.98 (1H, m), 7.35 (1H, m), 7.29 (2H, m), 4.03 (2H, s), 3.86 (3H, s), 3.82 (4H, m), 3.73 (1H, m), 1.76 (9H, s). (MH⁺, m/z) 392.

### Example 172

### 6-(1-(Benzo[d]thiazol-2-ylmethyl)azetidin-3-yl)-1-cyclohexyl-1H-pyrazolo[3,4-d]pyrimidin-4(5H)-one

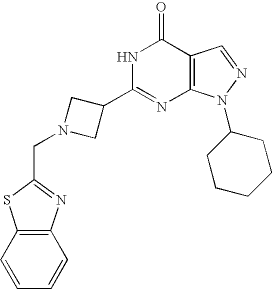

Prepared from Description 4 and 2-carbonyl-benzothiazole according to the procedure described in Example 171. ¹H NMR (400 MHz, CDCl₃): δ 10.66 (1H, br s), 8.10 (1H, s), 8.05 (1H, d, J=7.6 Hz), 7.91 (1H, d, J=7.6 Hz), 7.50 (1H, dd, J=7.6, 1.2 Hz), 7.41 (1H, dd, J=7.6, 1.2 Hz), 4.60 (1H, m), 4.17 (2H, s), 3.78 (4H, m), 3.58 (1H, m), 1.99 (6H, m), 1.75 (1H, m), 1.47 (2H, m), 1.27 (1H, m). (MH⁺, m/z) 421.

### Example 173

### 2-(3-(1-tert-Butyl-4-oxo-4,5-dihydro-1H-pyrazolo[3,4-d]pyrimidin-6-yl)azetidin-1-yl)-N-phenylacetamide

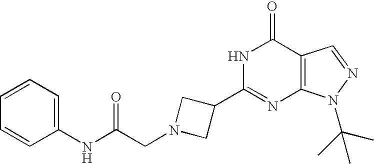

A suspension of Description 6 (75 mg, 0.265 mmol), 2-chloro-N-phenyl-acetamide (45 mg, 0.265 mmol) and potassium carbonate (77 mg, 0.557 mmol) in DMF (3 mL) was stirred at room temperature for 16 h. The reaction was diluted with MeOH and loaded onto a SCX (strong cation exchange) cartridge and eluted with MeOH to remove DMF. The crude product was eluted using 3.5 M methanolic ammonia and then purified by column chromatography, eluting with 5% 7N ammonia (in MeOH) in DCM to give the title compound as a white solid (36 mg, 36%). ¹H NMR (400 MHz, CDCl₃): δ 11.53 (1H, br s), 8.86 (1H, s), 8.04 (1H, s), 7.58 (2H, d, J=8 Hz), 7.34 (2H, d, J=8 Hz), 7.12 (1H, m), 3.95 (2H, m), 3.85 (1H, m), 3.76 (2H, m), 3.40 (2H, s), 1.81 (9H, s). (MH⁺, m/z) 381.

### Example 174

### 1-tert-Butyl-6-(1-(2-oxo-2-phenylethyl)azetidin-3-yl)-1H-pyrazolo[3,4-d]pyrimidin-4(5H)-one

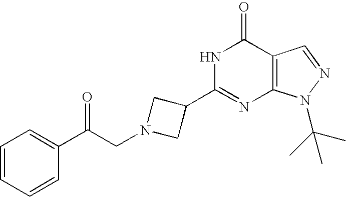

Prepared from Description 6 and bromoacetophenone according to the procedure of Example 173. ¹H NMR (400 MHz, CDCl₃): δ 10.65 (1H, br s), 8.02 (1H, s), 7.93 (2H, m), 7.59 (1H, m), 7.49 (2H, m), 4.04 (2H, s), 3.81 (2H, m), 3.76 (2H, m), 3.61 (1H, m), 1.77 (9H, s). (MH⁺, m/z) 366.

### Example 175

### 6-(1-((1H-Benzo[d]imidazol-2-yl)methyl)azetidin-3-yl)-1-tert-butyl-1H-pyrazolo[3,4-d]pyrimidin-4(5H)-one

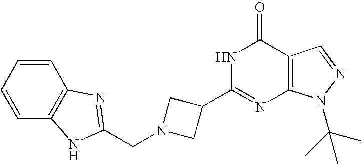

Prepared from Description 6 and 2-chloromethyl benzimidazole according to the procedure of Example 173. ¹H NMR (400 MHz, CDCl₃): 11.20 (1H, br s), 7.99 (1H, s), 7.58 (2H, m), 7.25 (2H, m), 4.05 (2H, s), 3.73 (4H, m), 3.63 (1H, m), 1.76 (9H, s). (MH⁺, m/z) 378.

### Example 176

### 6-(1-Benzylpiperidin-3-yl)-1-cyclohexyl-1H-pyrazolo[3,4-d]pyrimidin-4(5H)-one

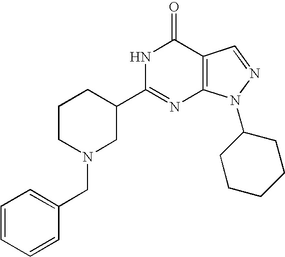

Prepared from Description 10 and benzyl bromide made in a manner analogous to that described in Example 173.

### Example 177

### N′-Cyano-3-(1-cyclohexyl-4-oxo-4,5-dihydro-1H-pyrazolo[3,4-d]pyrimidin-6-yl)-N-(4-fluorophenyl)azetidine-1-carboximidamide

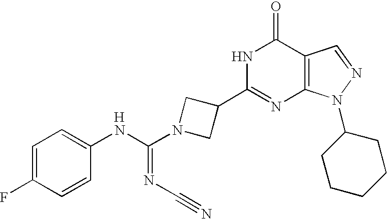

### Step 1: Phenyl N′-cyano-N-(4-fluorophenyl)carbamimidate

A mixture of diphenyl cyanocarbonimidate (245 mg, 1 mmol) and 4-fluoroaniline (113 mg, 1 mmol) in 2-propanol (3 mL) was stirred for 18 h at room temperature. A solid formed and was collected by filtration, washed with 2-propanol then 40-60° C. petrol, dried in vacuo and used without further purification (228 mg). ¹H NMR (400 MHz, CDCl₃): δ 8.90 (1H, br s), 7.30 (5H, m), 7.04-7.13 (4H, m). (MH⁺, m/z) 255.

### Step 2: N′-Cyano-3-(1-cyclohexyl-4-oxo-4,5-dihydro-1H-pyrazolo[3,4-d]pyrimidin-6-yl)-N-(4-fluorophenyl)azetidine-1-carboximidamide

A stirred suspension of Description 4 (51 mg, 0.16 mmol), the product of Step 1 (41 mg, 0.16 mmol) and triethylamine (0.1 mL, 0.75 mmol) in 2-propanol (2 mL) was heated at 50° C. for 16 h. Solvent was evaporated and the residue was pre-sorbed onto silica and purified using gradient column chromatography, eluting with 0-5% 17% ammonia (in MeOH) in DCM, to give the title compound as a white solid (49 mg). ¹H NMR (400 MHz, d6-DMSO): δ 8.94 (1H, br s), 8.08 (1H, s), 7.39 (2H, q), 7.20 (2H, t), 4.55-4.68 (5H, m), 4.00 (1H, m), 1.87-1.95 (6H, m), 1.75 (1H, m), 1.40-1.54 (2H, m), 1.24-1.34 (1H, m). (MH⁺, m/z) 434.

### Example 178

### N′-Cyano-3-(1-cyclohexyl-4-oxo-4,5-dihydro-1H-pyrazolo[3,4-d]pyrimidin-6-yl)-N-(cyclohexylmethyl)azetidine-1-carboximidamide

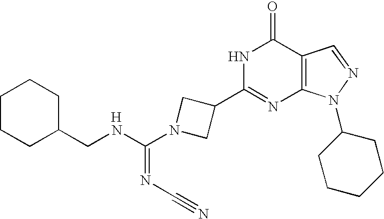

### Step 1: Phenyl N-cyano-3-(1-cyclohexyl-4-oxo-4,5-dihydro-1H-pyrazolo[3,4-d]pyrimidin-6-yl)azetidine-1-carbimidate

A mixture of Description 4 (105 mg, 0.338 mmol), diphenyl cyanocarbonimidate (82 mg 0.338 mmol) and triethylamine (0.15 mL, 1 mmol) in 2-propanol (2 mL) was stirred at room temperature for 18 h. The solution was pre-sorbed onto silica and purified using gradient column chromatography, eluting with 0-100% EtOAc in 40-60° C. petrol. The title compound was isolated as a solid (108 mg), which was used without further purification. ¹H NMR (400 MHz, CDCl₃): δ 12.74 (1H, br s), 8.06 (1H, s), 7.37 (2H, m), 7.10 (2H, m), 5.02-5.24 (2H, m), 4.68-4.76 (3H, m), 4.17 (1H, m), 1.9-2.0 (6H, m), 1.78 (1H, d), 1.66-1.48 (2H, m), 1.42-1.23 (1H, m). (MH⁺, m/z) 417.

### Step 2: N′-Cyano-3-(1-cyclohexyl-4-oxo-4,5-dihydro-1H-pyrazolo[3,4-d]pyrimidin-6-yl)-N-(cyclohexylmethyl)azetidine-1-carboximidamide

A mixture of the product of Step 1 (50 mg, 0.119 mmol) and cyclohexane methylamine (27 mg, 0.222 mmol) in 2-propanol (2 mL) was heated at 60° C. for 20 h. A solid formed and was collected by filtration, washed with 2-propanol, then 40-60° C. petrol, and dried in vacuo to give the title compound (31 mg). ¹H NMR (400 MHz, d6-DMSO): δ 12.20 (1H, br s), 8.05 (1H, s), 7.02 (1H, m), 4.60 (1H, m), 4.48 (4H, m), 3.98 (1H, m), 2.98 (2H, t), 2.00-1.84 (6H, m), 1.77-1.52 (6H, m), 1.58-1.50 (3H, m), 1.36-1.13 (4H, m), 0.88 (2H, m). (MH⁺, m/z) 436.

### Example 179

### 3-(1-tert-Butyl-4-oxo-4,5-dihydro-1H-pyrazolo[3,4-d]pyrimidin-6-yl)-N′-cyano-N-(4-fluorophenyl)azetidine-1-carboximidamide

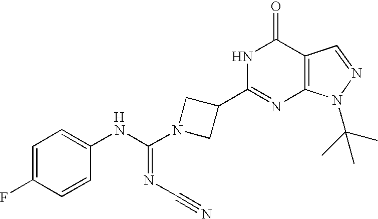

Prepared from Description 6 according to the procedure of Example 177.

### Example 180

### 1-Cyclohexyl-6-(1-(1-(cyclohexylmethylamino)-2-nitrovinyl)azetidin-3-yl)-1H=pyrazolo[3,4-d]pyrimidin-4(5H)-one

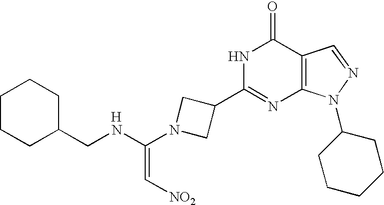

### Step 1: Cyclohexylmethyl-(1-methylsulfanyl-2-nitrovinyl)-amine

A mixture of 1,1-bis(methylthio)-2-nitroethylene (330 mg, 2 mmol), cyclohexane methylamine (226 mg, 2 mmol) and dry THF was heated in a sealed tube for 18 h. The solution was pre-sorbed onto silica and purified using gradient column chromatography, eluting with 0-30% EtOAc in 40-60° C. petrol. The title compound was isolated as a solid (303 mg), which was used without further purification. ¹H NMR (400 MHz, CDCl₃): δ 10.64 (1H, br s), 6.58 (1H, s), 3.47 (1H, s), 3.25 (2H, m), 2.40 (3H, m), 1.84-1.60 (6H, m), 1.25 (2H, m). (MH⁺, m/z) 230.

### Step 2: 1-Cyclohexyl-6-(1-(1-(cyclohexylmethylamino)-2-nitrovinyl)azetidin-3-yl)-1H-pyrazolo[3,4-d]pyrimidin-4(5H)-one

A mixture of Description 4 (35 mg, 0.16 mmol), the product of Step 1 (27 mg, 0.16 mmol) and triethylamine (0.1 mL, 0.75 mmol) in 2-propanol (2 mL) was heated at 60° C. for 18 h. The mixture was pre-sorbed onto silica and purified using gradient column chromatography, eluting with 0-6% 17% ammonia (in MeOH) in DCM. The title compound was isolated as a solid (35 mg). ¹H NMR (400 MHz, d6-DMSO): δ 12.00 (1H, br s), 10.10 (1H, s), 8.06 (1H, s), 6.26 (1H, s), 4.64 (1H, m), 4.58 (4H, m), 4.00 (1H, m), 3.24 (2H, m), 2.00-1.85 (6H, m), 1.78-1.65 (6H, m), 1.60-1.40 (3H, m), 1.34-1.14 (4H, m), 1.02 (2H, m). (MH⁺, m/z) 455.

### Example 181

### N-{2-[3-(1-Cyclohexyl-3-methyl-4-oxo-4,5-dihydro-1H-pyrazolo[3,4-d]pyrimidin-6-yl)-piperidine-1-carbonyl]phenyl}-methanesulfonamide

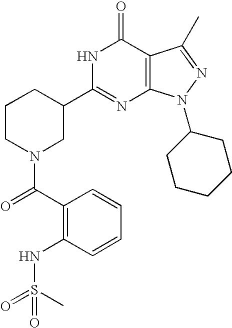

### Step 1: 5-Amino-1-cyclohexyl-3-methyl-1H-pyrazole-4-carbonitrile

A solution of (1-ethoxyethylidene)malononitrile (6.6 g, 49 mmol), cyclohexyl hydrazine dihydrochloride (9.2 g, 58 mmol) and triethylamine (28.5 mL, 200 mmol) in EtOH (150 mL) is heated at 85° C. for 16 hours. After this time the ethanol is evaporated, and the residue partitioned between diethyl ether (200 mL) and water (100 mL). The ether layer is separated, washed with brine (100 mL), dried (MgSO₄) and evaporated. 5-Amino-1-cyclohexyl-3-methyl-1H-pyrazole-4-carbonitrile (8.1 g,) is isolated as a solid that is used without any further purification. ¹H NMR (400 MHz, d6-DMSO): δ 1.15-1.22 (1H, m), 1.31-1.41 (2H, m), 1.62-1.82 (7H, m), 2.10 (3H, s), 3.96-4.02 (1H, m), 6.45 (2H, s).

### Step 2: 5-Amino-1-cyclohexyl-3-methyl-1H-pyrazole-4-carboxylic Acid Amide

Concentrated sulphuric acid (98%, 60 mL) is cooled to 0-5° C., and 5-amino-1-cyclohexyl-3-methyl-1H-pyrazole-4-carbonitrile (6.7 g, 33 mmol) is added portionwise with vigorous stirring. The mixture is stirred at 0-5° C. for 2 hours, then warmed to 45° C., and heated at this temperature for 30 minutes. After this time the mixture is poured onto crushed ice and basified by the addition of ammonium hydroxide solution. The product is extracted with ethyl acetate (4×150 mL) and the combined organic phases concentrated to dryness. The resulting solid is triturated with diethyl ether, and the colourless product (6.6 g, 90%) collected by filtration. 5-Amino-1-cyclohexyl-3-methyl-1H-pyrazole-4-carboxylic acid amide is used without any further purification. ¹H NMR (400 MHz, d6-DMSO): δ 1.16-1.24 (1H, m), 1.32-1.42 (2H, m), 1.65-1.83 (7H, m), 2.25 (3H, s), 3.94-3.99 (1H, m), 6.20 (2H, s), 6.47 (2H, br s). LCMS: Retention time 2.42 min corresponds to m/z 223.

### Step 3: 2-Methanesulfonylamino-benzoic Acid Ethyl Ester

To a solution of ethyl 2-aminobenzoate (7.2 g, 46 mmol) in DCM (80 mL) at 0° C., is added methanesulfonyl chloride (3.6 mL, 46 mmol) and pyridine (3.75 mL, 46 mmol). The reaction mixture was allowed to warm to room temperature, then stirred overnight. The solution was washed with water and brine, then dried (MgSO₄) and evaporated to afford the title ester (11.8 g) which was used without further purification. ¹H NMR (400 MHz, CDCl₃): δ 10.51 (1H, br s), 8.07 (1H, d, J=8 Hz), 7.74 (1H, d, J=8 Hz), 7.55 (1H, dd, J=8 Hz and 8 Hz), 7.13 (1H, dd, J=8 Hz and 8 Hz), 4.40 (2H, q, J=8 Hz), 3.06 (3H, s), 1.42 (3H, t, J=8 Hz).

### Step 4: 2-Methanesulfonylamino-benzoic Acid

To a solution of 2-methanesulfonylamino-benzoic acid ethyl ester (11.8 g, 48 mmol) in THF (100 mL) was added an aqueous solution of lithium hydroxide (2N, 50 mL). The mixture was stirred at room temperature for 18 h. The reaction mixture was evaporated and the residue treated with 2N hydrochloric acid. The resulting precipitate was extracted into diethyl ether (2×200 mL), and the combined organic layers washed with brine, dried and evaporated to afford the acid (8.55 g, 85%). ¹H NMR (400 MHz, CDCl₃): δ 10.10 (1H, br s), 8.14 (1H, d, J=8 Hz), 7.77 (1H, d, J=8 Hz), 7.63 (1H, dd, J=8 Hz and 8 Hz), 7.17 (1H, dd, J=8 Hz and 8 Hz), 3.10 (3H, s).

### Step 5: 1-(2-Methanesulfonylamino-benzoyl)-piperidine-3-carboxylic Acid Ethyl Ester

A solution of 2-methanesulfonylamino-benzoic acid (2 g, 9.3 mmol) in DCM (30 mL) was treated with 1,1′-carbonyldiimidazole (1.53 g, 9.3 mmol) and the solution stirred for 30 min. After this time piperidine-3-carboxylic acid ethyl ester (1.44 mL, 9.3 mmol) was added, and the mixture stirred for a further 1 h. The solution was then diluted with DCM, washed with saturated sodium bicarbonate, 2N hydrochloric acid and brine, then dried and evaporated. 1-(2-Methanesulfonylamino-benzoyl)-piperidine-3-carboxylic acid ethyl ester (2.58 g, 75%) was used without further purification.

### Step 6: 1-(2-Methanesulfonylamino-benzoyl)-piperidine-3-carboxylic Acid

A solution of 1-(2-methanesulfonylamino-benzoyl)-piperidine-3-carboxylic acid ethyl ester (2.58 g, 7.3 mmol) in THF (50 mL) was treated with an aqueous solution of lithium hydroxide (2N, 30 mL), and stirred vigorously for 48 h. After this time the solvent was evaporated and the residue acidified using 2N hydrochloric acid. The mixture was extracted into EtOAc, then the organic layer is separated, washed with brine, dried (MgSO₄) and evaporated. 1-(2-Methanesulfonylamino-benzoyl)-piperidine-3-carboxylic acid (1.62 g, 69%) is used in the subsequent reaction without further purification. ¹H NMR (400 MHz, CDCl₃): δ 7.86 (1H, s), 7.64 (1H, d, J=8 Hz), 7.40-7.44 (1H, m), 7.23-7.26 (1H, m), 7.15-7.19 (1H, m), 3.29-4.18 (4H, m), 3.08 (3H, s), 2.59-2.78 (1H, m), 1.51-2.0 (4H, m).

### Step 7: 1-(2-Methanesulfonylamino-benzoyl)-piperidine-3-carbonyl Chloride

A solution of 1-(2-methanesulfonylamino-benzoyl)-piperidine-3-carboxylic acid (1.56 g, 4.8 mmol) and triethylamine (1.3 mL, 9.1 mmol) in DCM (30 mL) was cooled to 0° C., and thionyl chloride (4.6 mL, 9.1 mmol) is added dropwise. The mixture was stirred for 2 h, then the solution was evaporated and the resultant 1-(2-methanesulfonylamino-benzoyl)-piperidine-3-carbonyl chloride used without further purification.

### Step 8: 5-Amino-1-cyclohexyl-3-methyl-1H-pyrazole-4-carboxylic acid Amide

Prepared from (2-ethoxyethlidene)malononitrile according to Description 1.

### Step 9: 1-(2-Methanesulfonylamino-benzoyl)-piperidine-3-carboxylic acid (4-carbamoyl-2-cyclohexyl-5-methyl-2H-pyrazol-3-yl) Amide

5-Amino-1-cyclohexyl-3-methyl-1H-pyrazole-4-carboxylic acid amide (Step 6, 800 mg, 3.4 mmol) was suspended in DCM (30 mL). The mixture was treated with triethylamine (4.7 mL, 3.4 mmol) followed by a solution of 1-(2-methanesulfonylamino-benzoyl)-piperidine-3-carbonyl chloride (Step 5, 1.2 g, 3.4 mmol) in DCM (14 mL). After addition of the acid chloride a clear solution formed. After stirring for a further 5 min a precipitate formed and the solution changed from brown to yellow. The solvent is removed, and the reaction mixture purified using column chromatography, eluting with hexane:EtOAc (2:1 then 1:2 then 0:1) followed by MeOH:EtOAc (5:95 then 10:90), to give 1-(2-methanesulfonylamino-benzoyl)-piperidine-3-carboxylic acid (4-carbamoyl-2-cyclohexyl-5-methyl-2H-pyrazol-3-yl) amide (0.32 g, 18%).

### Step 10: N-{2-[3-(1-Cyclohexyl-3-methyl-4-oxo-4,5-dihydro-1H-pyrazolo[3,4-d]pyrimidin-6-yl)-piperidine-1-carbonyl]phenyl}-methanesulfonamide

1-(2-Methanesulfonylamino-benzoyl)-piperidine-3-carboxylic acid (4-carbamoyl-2-cyclohexyl-5-methyl-2H-pyrazol-3-yl) amide (316 mg, 0.6 mmol) was dissolved in EtOH (20 mL) and 1N sodium hydroxide solution (10 mL). The mixture was heated at 100° C. for 18 h, then the solution cooled to room temperature, the solvents evaporated, and the residue treated with 2N hydrochloric acid. On addition of the aqueous acid a precipitate formed. The mixture is extracted with ethyl acetate (2×30 mL), and the combined organic layers washed with brine, dried (MgSO₄) and evaporated. The residue was purified using column chromatography, eluting with hexane:ethyl acetate (2:3 then 1:4 then 0:1), to give N-{2-[3-(1-cyclohexyl-3-methyl-4-oxo-4,5-dihydro-1H-pyrazolo[3,4-d]pyrimidin-6-yl)-piperidine-1-carbonyl]phenyl}-methanesulfonamide (101 mg, 55%). ¹H NMR (400 MHz, CDCl₃): δ 11.38 (1H, br s), 8.00 (1H, br s), 7.67-7.52 (1H, m), 7.47-7.39 (1H, m), 7.34-7.28 (1H, m), 7.20-7.10 (1H, m), 4.90 3.74 (2H, br m), 3.45 (1H, dd, J=12 Hz and 12 Hz), 3.05-2.89 (1H, m), 3.20 (3H, s), 2.55 (3H, s), 2.31-2.20 (1H, m), 2.10-1.80 (8H, m), 2.10-1.58 (3H, m), 1.51-1.20 (4H, m). (MH⁺, m/z) 512.

### Example 182

### 3-(1-Cyclohexyl-4-oxo-4,5-dihydro-1H-pyrazolo[3,4-d]pyrimidin-6-yl)-azetidine-1-carboxylic acid piperidin-3-ylamide trifluoroacetate

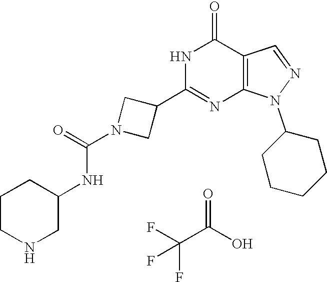

A 20% solution of trifluoroacetic acid in DCM (5 mL) was added to 3-{[3-(1-cyclohexyl-4-oxo-4,5-dihydro-1H-pyrazolo[3,4-d]pyrimidin-6-yl)-azetidine-1-carbonyl]-amino}-piperidine-1-carboxylic acid tert-butyl ester (Example 26, 28 mg, 0.056 mmol). The mixture was stirred for 1 h then the solvents were removed in vacuo to give the required product as a white solid (29 mg, 0.056 mmol). ¹H NMR (400 MHz, D₂O): δ 8.05 (1H, s), 4.34-4.20 (4H, m), 3.95-3.78 (2H, m), 3.41-3.25 (2H, m), 2.95-2.75 (2H, m), 2.00-1.61 (10H, m), 1.60-1.35 (3H, m), 1.28-1.15 (1H, m). MS (MH⁺, m/z) 400.

### Example 183

### 3-(1-Cyclohexyl-4-oxo-4,5-dihydro-1H-pyrazolo[3,4-d]pyrimidin-6-yl)-azetidine-1-carboxylic acid (1-isopropyl-piperidin-3-yl)-amide

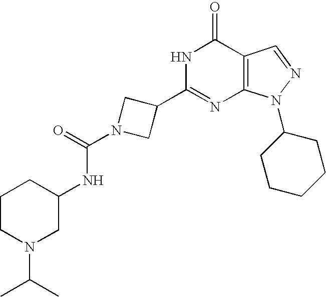

The title compound was prepared from 3-(1-cyclohexyl-4-oxo-4,5-dihydro-1H-pyrazolo[3,4-d]pyrimidin-6-yl)-azetidine-1-carboxylic acid piperidin-3-ylamide trifluoroacetate (0.056 mmol, 0.029 g) and acetone according to the procedure of Description 17. The product was isolated as a white solid (15 mg, 62%). ¹H NMR (400 MHz, d6-DMSO): δ 12.00 (1H, br s), 7.93 (1H, s), 6.03 (1H, d, J=8 Hz), 4.55-4.45 (1H, m), 4.05-3.95 (4H, m), 3.78-3.70 (1H, m), 3.48-3.35 (1H, m), 2.68-2.50 (3H, m), 2.40-2.30 (2H, m), 1.98-1.72 (6H, m), 1.68-1.50 (3H, m), 1.45-1.05 (5H, m), 0.90-0.83 (6H, m). MS (MH⁺, m/z) 442.

### Example 184

### 3-(1-Cyclohexyl-4-oxo-4,5-dihydro-1H-pyrazolo[3,4-d]pyrimidin-6-yl)-N-((1-isopropylpiperidin-4-yl)methyl)azetidine-1-carboxamide

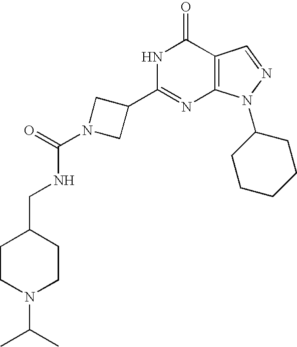

Prepared from 4-({[3-(1-cyclohexyl-4-oxo-4,5-dihydro-1H-pyrazolo[3,4-d]pyrimidin-6-yl)-azetidine-1-carbonyl]-amino}-methyl)-piperidine-1-carboxylic acid tert-butyl ester (made in a manner analogous to Example 26) following the procedures described in Example 182 and 183. ¹H NMR (400 MHz, CDCl₃): δ 8.08 (1H, s), 4.66 (1H, m), 4.34 (4H, m), 3.91 (1H, m), 3.15 (2H, t, J=6.4 Hz), 2.89 (2H, m), 2.70 (1H, m), 2.11 (2H, m), 1.97 (6H, m), 1.72 (3H, m), 1.52 (3H, m), 1.37 (3H, m), 1.02 (6H, d, J=6.6). MS (MH⁺, m/z) 456.

### Example 185

### 3-[1-(1-Isopropyl-azetidin-3-yl)-4-oxo-4,5-dihydro-1H-pyrazolo[3,4-d]pyrimidin-6-yl]-azetidine-1-carboxylic acid (4-fluoro-phenyl)-amide

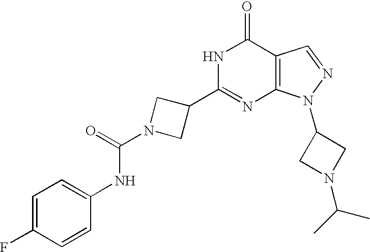

### Step 1: 1-Benzhydryl-azetidin-3-one

Sulfur trioxide pyridine complex (19.96, g, 125.40 mmol) was added over 10 min to a solution of 1-benzhydryl-azetidin-3-ol (5.0 g, 21.0 mmol) and triethylamine (14.60 mL, 104.60 mmol) in THF (20 mL) and DMSO (50 mL). The yellow solution was stirred for 2 h then poured into water (50 mL). The aqueous phase was extracted with 1:1 EtOAc:hexanes (4×50 mL). The combined organics were washed with water (50 mL), dried over MgSO₄ then concentrated in vacuo to give the product as a pale brown oil, which solidified on standing (18.56 mmol, 4.40 g, 88%). ¹H NMR (400 MHz, CDCl₃): δ 7.50-7.45 (4H, m), 7.33-7.18 (6H, m), 4.59 (1H, s), 4.00 (s, 4H).

### Step 2: N′-(1-Benzhydryl-azetidin-3-ylidene)-hydrazinecarboxylic Acid Tert-Butyl Ester

1-Benzhydryl-azetidin-3-one (Step 1, 14.93 g, 63.00 mmol) and tert-butyl carbazate (8.32 g, 63.00 mmol) were mixed in MeOH (150 mL), and AcOH (7.2 mL, 126.00 mmol) was added. The mixture was stirred for 18 h then the solvents were removed in vacuo. The residue was dissolved in DCM (50 mL) and the organics were washed with 1M NaOH (50 mL), dried with MgSO₄ and concentrated in vacuo to give a pale brown solid. This was triturated with diethyl ether then filtered and dried to give the product as a white powder (51.60 mmol, 18.12 g, 82%). ¹H NMR (400 MHz, CDCl₃): δ 7.45-7.40 (4H, m), 7.33-7.18 (6H, m), 4.52 (1H, s), 4.00-3.97 (2H, m), 3.88-3.85 (2H, m), 1.46 (9H, s).

### Step 3: N′-(1-Benzhydryl-azetidin-3-yl)-hydrazinecarboxylic Acid Tert-Butyl Ester

N′-(1-Benzhydryl-azetidin-3-ylidene)-hydrazinecarboxylic acid tert-butyl ester (18.66 g, 53.20 mmol) was dissolved in AcOH (150 mL) then sodium cyanoborohydride (3.31 g, 53.20 mmol) was added. The mixture was stirred for 5 h then concentrated in vacuo to about ⅓ volume. 1M NaOH was added to pH 7 then the mixture was extracted with DCM (4×100 mL). The combined organics were dried over MgSO₄ and then concentrated in vacuo to give a white solid. This was triturated with diethyl ether then filtered and dried to give the product as a white powder (38.90 mmol, 13.72 g, 73%). ¹H NMR (400 MHz, CDCl₃): δ 7.41-7.36 (4H, m), 7.28-7.15 (6H, m), 6.10 (1H, br s), 4.34 (1H, s), 3.79-3.70 (1H, m), 3.37-3.32 (2H, m), 2.95-2.91 (2H, m), 1.43 (9H, s).

### Step 4: (1-Benzhydryl-azetidin-3-yl)-hydrazine Trihydrochloride

N′-(1-Benzhydryl-azetidin-3-yl)-hydrazinecarboxylic acid tert-butyl ester (2.83 mmol) was dissolved in 4M HCl in dioxane (10 mL). The mixture was stirred for 3 h then the solvents were removed in vacuo to give the product as a cream solid (2.70 mmol, 0.98 g, 95%). ¹H NMR (400 MHz, d6-DMSO): δ 12.80-12.50 (1H, br m), 9.70-9.50 (3H, br m), 7.77-7.65 (4H, m), 7.50-7.36 (6H, m), 6.12-5.90 (1H, br m), 4.30-3.95 (5H, m). MS (MH⁺, m/z) 254.

### Step 5: 5-Amino-1-(1-benzhydryl-azetidin-3-yl)-1H-pyrazole-4-carboxylic Acid Amide

(1-Benzhydryl-azetidin-3-yl)-hydrazine trihydrochloride (0.98 g, 2.70 mmol), ethoxymethylidene malononitrile (0.30 g, 2.45 mmol) and triethylamine (1.70 mL, 12.25 mmol) were mixed in EtOH (10 mL) and heated at 90° C. for 16 h. The solvents were removed in vacuo and the residue was partitioned between water (10 mL) and EtOAc (10 mL). The aqueous phase was extracted with EtOAc (3×10 mL) then the combined organics were dried over MgSO₄ and concentrated in vacuo to give a brown oil. This was dissolved and evaporated from EtOH (2×) and diethyl ether to give the crude nitrile product as a brown foam. This foam was added portionwise to concentrated H₂SO₄ (5 mL) at 0° C. The resulting mixture was stirred for 2 h then heated to 45° C. for 1 hour. The cooled mixture was poured onto ice and basified with 0.88 ammonia solution (10 mL). The aqueous phase was extracted with EtOAc (4×30 mL). The combined organics were dried over MgSO₄ then concentrated in vacuo to give the title compound as a brown foam (1.44 mmol, 0.50 g, 59%). ¹H NMR (400 MHz, d6-DMSO): δ 7.77 (1H, s), 7.58-7.45 (4H, m), 7.39-7.20 (6H, m), 6.37 (2H, s), 4.98-4.90 (1H, m), 4.61 (1H, s), 3.63-3.55 (2H, m). MS (MH⁺, m/z) 348.

### Step 6: 3-(5-Amino-4-carbamoyl-pyrazol-1-yl)-azetidine-1-carboxylic Acid Tert-Butyl Ester

5-Amino-1-(1-benzhydryl-azetidin-3-yl)-1H-pyrazole-4-carboxylic acid amide (3.50 g, 10.09 mmol) was dissolved in EtOH (50 mL) and 1M HCl (30 mL). Palladium hydroxide on carbon (20% wt., 3.50 g) was added and the mixture was stirred under a hydrogen atmosphere (200 psi) for 6 h. The mixture was filtered and the solvent removed in vacuo. The residue was dissolved and evaporated from EtOH (3×) to give a yellow solid. This was dissolved in 1M NaOH (50.45 mL, 50.45 mmol) and THF (10 mL) and cooled to 0° C. Di-tert-butyldicarbonate (2.31 g, 10.59 mmol) was added and mixture was stirred for 6 h. The organics were separated and the aqueous phase was extracted with EtOAc (3×50 mL). The combined organics were dried over MgSO₄ and concentrated in vacuo to give a yellow solid. This was purified by gradient column chromatography (silica gel, 5-10% MeOH in DCM) to give the product as a white solid (2.85 mmol, 0.80 g, 28%). ¹H NMR (400 MHz, d6-DMSO): δ 7.81 (1H, s), 6.30 (2H, s), 5.10 (1H, br), 4.27-4.08 (4H, m), 1.44 (9H, s).

### Step 7: 3-[6-(1-Benzhydryl-azetidin-3-yl)-4-oxo-4,5-dihydro-pyrazolo[3,4-d]pyrimidin-1-yl]-azetidine-1-carboxylic Acid Tert-Butyl Ester

Prepared according to Description 3, Step 2 from 3-(5-amino-4-carbamoyl-pyrazol-1-yl)-azetidine-1-carboxylic acid tert-butyl ester (0.80 g, 2.85 mmol) to give the product, after gradient column chromatography (silica, 0-4% MeOH in DCM), as an orange foam (1.30 mmol, 0.74 g, 46%). ¹H NMR (400 MHz, CDCl₃): δ 11.00 (1H, br s), 8.16 (1H, s), 7.48-7.40 (4H, m), 7.35-7.18 (6H, m), 5.58-5.49 (1H, m), 4.50-4.45 (3H, m), 4.40-4.35 (2H, m), 3.55-3.39 (5H, m), 1.47 (9H, s).

### Step 8: 6-(1-Benzhydryl-azetidin-3-yl)-1-(1-isopropyl-azetidin-3-yl)-1,5-dihydro-pyrazolo[3,4-d]pyrimidin-4-one

Prepared from 3-[6-(1-benzhydryl-azetidin-3-yl)-4-oxo-4,5-dihydro-pyrazolo[3,4-d]pyrimidin-1-yl]-azetidine-1-carboxylic acid tert-butyl ester (0.74 g, 1.43 mmol) by treatment with 50% TFA in DCM then by following the procedure of Description 17 to give the product as a white solid after gradient column chromatography (silica, 0-4% MeOH in DCM) (0.67 mmol, 0.31 g, 46%). ¹H NMR (400 MHz, CDCl₃): δ 11.20 (1H, br s), 8.13 (1H, s), 7.49-7.40 (4H, m), 7.36-7.18 (6H, m), 5.57-5.48 (1H, m), 4.46 (1H, s), 4.03-3.95 (2H, m), 3.26-3.66 (2H, m), 3.57-3.50 (3H, m), 3.45-3.40 (2H, m), 2.78-2.67 (1H, m), 1.08-1.00 (6H, m).

### Step 9: 6-Azetidin-3-yl-1-(1-isopropyl-azetidin-3-yl)-1,5-dihydro-pyrazolo[3,4-d]pyrimidin-4-one dihydrochloride

Prepared from 6-(1-benzhydryl-azetidin-3-yl)-1-(1-isopropyl-azetidin-3-yl)-1,5-dihydro-pyrazolo[3,4-d]pyrimidin-4-one (0.67 mmol, 0.306 g) according to the procedure of Description 3, Step 3 to give the product as a cream solid (0.53 mmol, 0.189 g, 78%). ¹H NMR (400 MHz, d6-DMSO): δ 12.45 (1H, s), 11.98 and 11.10 (1H, br s), 9.70-9.25 (2H, br m), 8.32 and 8.30 (1H, s), 5.88-5.65 (1H, m), 4.80-4.12 (9H, m). 1.33-1.23 (6H, m). MS (MH⁺, m/z) 289.

### Step 10: 3-[1-(1-Isopropyl-azetidin-3-yl)-4-oxo-4,5-dihydro-1H-pyrazolo[3,4-d]pyrimidin-6-yl]-azetidine-1-carboxylic acid (4-fluoro-phenyl)-amide

4-Fluorophenyl isocyanate (0.034 mL, 0.30 mmol) was added dropwise to a solution of 6-azetidin-3-yl-1-(1-isopropyl-azetidin-3-yl)-1,5-dihydro-pyrazolo[3,4-d]pyrimidin-4-one dihydrochloride (0.25 mmol, 0.090 g) and triethylamine (0.077 mL, 0.55 mmol) in DCM (3 mL) at 0° C. The mixture was allowed to warm to room temperature and was stirred for 18 h then diluted with DCM (10 mL). The organics were washed with water (10 mL), dried over MgSO₄ and concentrated to give a clear residue. This was purified by gradient column chromatography (silica, 5-15% MeOH in DCM) to give the product as a white solid (0.16 mmol, 0.068 g, 64%). ¹H NMR (400 MHz, d6-DMSO): δ 12.25 (1H, s), 8.61 (1H, s), 8.13 (1H, s), 7.60-7.51 (2H, m), 7.15-7.06 (2H, m), 5.38-5.25 (1H, m), 4.32-4.22 (4H, m), 3.97-3.88 (1H, m), 3.82-3.72 (2H, m), 2.61-2.57 (1H, m), 0.97-0.87 (6H, m). MS (MH⁺, m/z) 426.

### 2. Biological Assays

### 2.1 PDE1A Assay

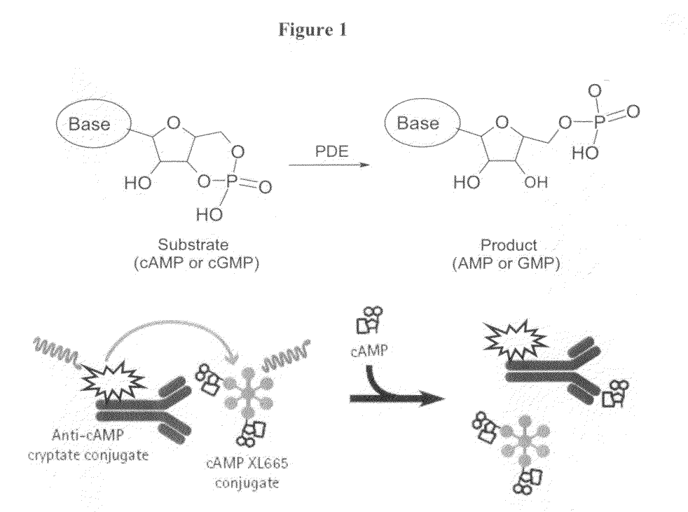

For the primary screening an assay using the cAMP dynamic HTRF® kit from Cisbio (catnr 62AM2PEB) is used. Its principle is based on HTRF® technology (Homogeneous Time-Resolved Fluorescence). The method is a competitive immunoassay between native cAMP and the cAMP labeled with XL665. The tracer binding is visualized by a monoclonal antibody against cAMP, labeled with Cryptate. The specific signal (i.e. energy transfer) is inversely proportional to the concentration of cAMP in the sample (see FIG. 1).

For the enzymatic reaction, a mixture is made of 20 μL with purified PDE1A enzyme, 100 nM cAMP and the compound (10 μM in a final concentration of 1% DMSO) in a black 384-plate. The reaction buffer is Tris 20 mM pH 7.4, 4 μg/mLl calmodulin, 3 mM MgCl₂, 1.5 mM CaCl₂, 0.2 mg/mL BSA and 0.001% Brij-35®. After an incubation of 30 minutes at room temperature, the reaction is stopped by the addition of 10 μL labelled cAMP-XL665 and 10 μL anti-cAMP-Cryptate. After 1 hour incubation at room temperature, the readout is performed on the Envision (excitation 360 nm; emission donor 615 nm; emission acceptor 665 nm).

PDE1A hydrolyses cAMP into 5′AMP; this low cAMP concentration will result in a high signal. A PDE1A inhibitor will result in a decrease of the signal.

As a positive control we used 10 μM Vardenafil (100% inhibition), as negative control we used 1% DMSO (0% inhibition), as variable control 10 μM Zaprinast (+/−50% inhibition) and as negative control compound 10 μM Ro-20-1724 (0% inhibition). The positive and negative control are used to calculate z′ and PIN values.

All compounds are screened in single at 10 μM. The hit criterium is set at PIN 50 (50% inhibition).

For the dose response and further screening, we used the Cyclic Nucleotide

Phosphodiesterase Assay Kit from Biomol, a colorimetric, non-radioactive assay. The basis for the assay is the cleavage of cAMP by PDE1A. The 5′AMP is further cleaved into the nucleoside and phosphate by the enzyme 5′-nucleotidase (catnr KI-307). The phosphate released due to enzymatic cleavage is quantified using BIOMOL GREEN™ reagent (catnr AK-111) in a modified Malachite Green assay^{1,2}. A PDE1A inhibitor will result in a decrease of the signal.

For the enzymatic reaction a mix of 25 μL with purified PDE1A enzyme, 100 μM cAMP, 5′Nucleotidase and the compound is made in a clear 384-plate. The reaction buffer is Tris 20 mM pH 7.4, 4 μg/mL calmodulin, 3 mM MgCl₂, 1.5 mM CaCl₂, 0.2 mg/mL BSA and 0.001% Brij-35. After an incubation of 45 minutes at 37° C., the reaction is stopped by the addition of 50 μL BIOMOL GREEN™ reagent. After 30 minutes incubation at room temperature, the readout is performed on the Envision (absorption at 615 nm).

All compounds are tested in duplicate starting from 20M followed by a 1/3 serial dilution, 8 points (20 μM-6.67 μM-2.22 μM-740 nM-247 nM-82 nM-27 nM-9 nM) in a final concentration of 1% DMSO. As the positive dose response control compound Vardenafil is used. For the calculation of z′ and PIN values 10 μM Vardenafil is used as positive control (100% inhibition) and 1% DMSO as negative control (0% inhibition).

The following compounds have been or can be prepared according to the synthetic methods described above. For the purpose of Table 7 below, PDE1A activity of each compound, which can be determined using the assay methods described herein, is expressed as follows:

++++ compound exhibited PDE1A IC₅₀ 1-100 in M

+++ compound exhibited PDE1A IC₅₀ 101-500 nM

++ compound exhibited PDE1A IC₅₀ 501-1000 nM

+ compound exhibited PDE1A IC₅₀ 1001-1500 nM

TABLE 7

| | | PDE1A |
|---|---|---|
| EXAMPLE # | IUPAC_Name | IC₅₀ |
| 1 | N-(benzo[d][1,3]dioxol-5-yl)-3-(1-tert-butyl-3-methyl-4-oxo-4,5- | ++++ |
| | dihydro-1H-pyrazolo[3,4-d]pyrimidin-6-yl)azetidine-1-carboxamide | |
| 2 | 3-(1-cyclohexyl-4-oxo-4,5-dihydro-1H-pyrazolo[3,4-d]pyrimidin-6- | ++++ |
| | yl)-N-(4-(4-isopropylpiperazin-1-yl)phenyl)azetidine-1-carboxamide | |
| 3 | 3-(1-cyclohexyl-4-oxo-4,5-dihydro-1H-pyrazolo[3,4-d]pyrimidin-6- | ++++ |
| | yl)-N-(4-(4-methylpiperazin-1-yl)phenyl)azetidine-1-carboxamide | |
| 4 | 3-(1-cyclohexyl-4-oxo-4,5-dihydro-1H-pyrazolo[3,4-d]pyrimidin-6- | ++++ |
| | yl)-N-(3,4-dimethylphenyl)azetidine-1-carboxamide | |
| 5 | 3-(1-cyclohexyl-4-oxo-4,5-dihydro-1H-pyrazolo[3,4-d]pyrimidin-6- | ++++ |
| | yl)-N-(cyclohexylmethyl)azetidine-1-carboxamide | |
| 6 | 3-(1-tert-butyl-3-methyl-4-oxo-4,5-dihydro-1H-pyrazolo[3,4- | ++++ |
| | d]pyrimidin-6-yl)-N-phenylazetidine-1-carboxamide | |
| 7 | 3-(1-cyclohexyl-4-oxo-4,5-dihydro-1H-pyrazolo[3,4-d]pyrimidin-6- | ++++ |
| | yl)-N-(3-(dimethylamino)phenyl)azetidine-1-carboxamide | |
| 8 | N-(3-chlorophenyl)-3-(1-cyclohexyl-4-oxo-4,5-dihydro-1H- | ++++ |
| | pyrazolo[3,4-d]pyrimidin-6-yl)azetidine-1-carboxamide | |
| 9 | 3-(1-cyclohexyl-4-oxo-4,5-dihydro-1H-pyrazolo[3,4-d]pyrimidin-6- | ++++ |
| | yl)-N-(4-(dimethylamino)phenyl)azetidine-1-carboxamide | |
| 10 | 3-(1-cyclohexyl-4-oxo-4,5-dihydro-1H-pyrazolo[3,4-d]pyrimidin-6- | ++++ |
| | yl)-N-(4-morpholinophenyl)azetidine-1-carboxamide | |
| 11 | 3-(1-cyclohexyl-4-oxo-4,5-dihydro-1H-pyrazolo[3,4-d]pyrimidin-6- | ++++ |
| | yl)-N-phenylazetidine-1-carboxamide | |
| 12 | 3-(1-tert-butyl-3-methyl-4-oxo-4,5-dihydro-1H-pyrazolo[3,4- | ++++ |
| | d]pyrimidin-6-yl)-N-(4-fluorophenyl)azetidine-1-carboxamide | |
| 13 | 3-(1-cyclohexyl-4-oxo-4,5-dihydro-1H-pyrazolo[3,4-d]pyrimidin-6- | ++++ |
| | yl)-N-(4-methoxyphenyl)azetidine-1-carboxamide | |
| 14 | 3-(1-cyclohexyl-3-methyl-4-oxo-4,5-dihydro-1H-pyrazolo[3,4- | ++++ |
| | d]pyrimidin-6-yl)-N-phenylazetidine-1-carboxamide | |
| 15 | 3-(1-tert-butyl-3-methyl-4-oxo-4,5-dihydro-1H-pyrazolo[3,4- | ++++ |
| | d]pyrimidin-6-yl)-N-cyclohexylazetidine-1-carboxamide | |
| 16 | 3-(1-tert-butyl-4-oxo-4,5-dihydro-1H-pyrazolo[3,4-d]pyrimidin-6-yl)- | ++++ |
| | N-(4-fluorophenyl)azetidine-1-carboxamide | |
| 17 | 3-(1-tert-butyl-3-methyl-4-oxo-4,5-dihydro-1H-pyrazolo[3,4- | ++++ |
| | d]pyrimidin-6-yl)-N-(4-fluorobenzyl)azetidine-1-carboxamide | |
| 18 | 3-(1-cyclohexyl-4-oxo-4,5-dihydro-1H-pyrazolo[3,4-d]pyrimidin-6- | ++++ |
| | yl)-N-(4-isopropylphenyl)azetidine-1-carboxamide | |
| 19 | 3-(1-cyclohexyl-4-oxo-4,5-dihydro-1H-pyrazolo[3,4-d]pyrimidin-6- | ++++ |
| | yl)-N-(4-fluorophenyl)azetidine-1-carboxamide | |
| 20 | 3-(1-cyclohexyl-4-oxo-4,5-dihydro-1H-pyrazolo[3,4-d]pyrimidin-6- | ++++ |
| | yl)-N-(3,4-difluorophenyl)azetidine-1-carboxamide | |
| 21 | 3-(1-cyclohexyl-3-methyl-4-oxo-4,5-dihydro-1H-pyrazolo[3,4- | ++++ |
| | d]pyrimidin-6-yl)-N-(4-fluorophenyl)azetidine-1-carboxamide | |
| 22 | N-benzyl-3-(1-tert-butyl-3-methyl-4-oxo-4,5-dihydro-1H- | ++++ |
| | pyrazolo[3,4-d]pyrimidin-6-yl)azetidine-1-carboxamide | |
| 23 | N-cyclohexyl-3-(1-cyclohexyl-4-oxo-4,5-dihydro-1H-pyrazolo[3,4- | ++++ |
| | d]pyrimidin-6-yl)azetidine-1-carboxamide | |
| 24 | N-benzyl-3-(1-cyclohexyl-3-methyl-4-oxo-4,5-dihydro-1H- | ++++ |
| | pyrazolo[3,4-d]pyrimidin-6-yl)azetidine-1-carboxamide | |
| 25 | 3-(1-cyclohexyl-4-oxo-4,5-dihydro-1H-pyrazolo[3,4-d]pyrimidin-6- | ++++ |
| | yl)-N-(4-(trifluoromethoxy)phenyl)azetidine-1-carboxamide | |
| 26 | tert-butyl 3-(3-(1-cyclohexyl-4-oxo-4,5-dihydro-1H-pyrazolo[3,4- | ++++ |
| | d]pyrimidin-6-yl)azetidine-1-carboxamido)piperidine-1-carboxylate | |
| 27 | 3-(1-cyclohexyl-3-methyl-4-oxo-4,5-dihydro-1H-pyrazolo[3,4- | ++++ |
| | d]pyrimidin-6-yl)-N-(4-fluorobenzyl)azetidine-1-carboxamide | |
| 28 | 3-(1-cyclohexyl-3-methyl-4-oxo-4,5-dihydro-1H-pyrazolo[3,4- | ++++ |
| | d]pyrimidin-6-yl)-N-(4-methoxybenzyl)azetidine-1-carboxamide | |
| 29 | N-(2-(difluoromethoxy)phenyl)-3-(1-(4-fluorophenyl)-3-methyl-4- | ++++ |
| | oxo-4,5-dihydro-1H-pyrazolo[3,4-d]pyrimidin-6-yl)azetidine-1- | |
| | carboxamide | |
| 30 | 3-(1-cyclohexyl-4-oxo-4,5-dihydro-1H-pyrazolo[3,4-d]pyrimidin-6- | ++++ |
| | yl)-N-(4-fluorobenzyl)azetidine-1-carboxamide | |
| 31 | N-benzyl-3-(1-cyclohexyl-4-oxo-4,5-dihydro-1H-pyrazolo[3,4- | ++++ |
| | d]pyrimidin-6-yl)azetidine-1-carboxamide | |
| 32 | 3-(1-cyclohexyl-4-oxo-4,5-dihydro-1H-pyrazolo[3,4-d]pyrimidin-6- | ++++ |
| | yl)-N-cyclopentylazetidine-1-carboxamide | |
| 33 | N-(4-cyanophenyl)-3-(1-cyclohexyl-4-oxo-4,5-dihydro-1H- | ++++ |
| | pyrazolo[3,4-d]pyrimidin-6-yl)azetidine-1-carboxamide | |
| 34 | N-butyl-3-(1-cyclohexyl-4-oxo-4,5-dihydro-1H-pyrazolo[3,4- | ++++ |
| | d]pyrimidin-6-yl)azetidine-1-carboxamide | |
| 35 | 3-(1-cyclohexyl-4-oxo-4,5-dihydro-1H-pyrazolo[3,4-d]pyrimidin-6- | ++++ |
| | yl)-N-(4-(trifluoromethyl)phenyl)azetidine-1-carboxamide | |
| 37 | 3-(1-cyclohexyl-4-oxo-4,5-dihydro-1H-pyrazolo[3,4-d]pyrimidin-6- | ++++ |
| | yl)-N-(3,4-dichlorobenzyl)azetidine-1-carboxamide | |
| 38 | N-tert-butyl-3-(1-cyclohexyl-4-oxo-4,5-dihydro-1H-pyrazolo[3,4- | ++++ |
| | d]pyrimidin-6-yl)azetidine-1-carboxamide | |
| 39 | 3-(1-cyclohexyl-3-methyl-4-oxo-4,5-dihydro-1H-pyrazolo[3,4- | ++++ |
| | d]pyrimidin-6-yl)-N-(3,4-dichlorobenzyl)azetidine-1-carboxamide | |
| 40 | 3-(1-cyclohexyl-4-oxo-4,5-dihydro-1H-pyrazolo[3,4-d]pyrimidin-6- | ++++ |
| | yl)-N-(1-(methylsulfonyl)piperidin-4-yl)azetidine-1-carboxamide | |
| 41 | tert-butyl 4-(3-(1-cyclohexyl-4-oxo-4,5-dihydro-1H-pyrazolo[3,4- | ++++ |
| | d]pyrimidin-6-yl)azetidine-1-carboxamido)piperidine-1-carboxylate | |
| 42 | (S)-3-(1-cyclohexyl-3-methyl-4-oxo-4,5-dihydro-1H-pyrazolo[3,4- | ++++ |
| | d]pyrimidin-6-yl)-N-(1-phenylethyl)azetidine-1-carboxamide | |
| 43 | 3-(1-cyclohexyl-4-oxo-4,5-dihydro-1H-pyrazolo[3,4-d]pyrimidin-6- | ++++ |
| | yl)-N-(3,3,3-trifluoropropyl)azetidine-1-carboxamide | |
| 44 | 1-cyclohexyl-6-(1-(3,4-dimethoxyphenylsulfonyl)piperidin-3-yl)-1H- | ++++ |
| | pyrazolo[3,4-d]pyrimidin-4(5H)-one | |
| 45 | 1-cyclohexyl-6-(1-(3-(dimethylamino)benzoyl) | +++ |
| | piperidin-3-yl)-1H-pyrazolo[3,4-d]pyrimidin-4(5H)-one | |
| 46 | 6-(1-(4-methoxyphenylsulfonyl)piperidin-3-yl)-1-phenyl-1H- | +++ |
| | pyrazolo[3,4-d]pyrimidin-4(5H)-one | |
| 47 | 3-(1-cyclohexyl-4-oxo-4,5-dihydro-1H-pyrazolo[3,4-d]pyrimidin-6- | +++ |
| | yl)-N-(tetrahydro-2H-pyran-4-yl)azetidine-1-carboxamide | |
| 48 | 1-cyclohexyl-6-(1-(2-methoxybenzoyl) | +++ |
| | piperidin-3-yl)-1H-pyrazolo[3,4-d]pyrimidin-4(5H)-one | |
| 49 | 6-(1-(benzo[d][1,3]dioxole-4-carbonyl)piperidin-3-yl)-1-cyclohexyl- | +++ |
| | 1H-pyrazolo[3,4-d]pyrimidin-4(5H)-one | |
| 50 | N-cyclohexyl-3-(1-(4-fluorophenyl)-3-methyl-4-oxo-4,5-dihydro-1H- | +++ |
| | pyrazolo[3,4-d]pyrimidin-6-yl)azetidine-1-carboxamide | |
| 51 | 1-cyclohexyl-6-(1-(4-methoxyphenylsulfonyl)piperidin-3-yl)-1H- | +++ |
| | pyrazolo[3,4-d]pyrimidin-4(5H)-one | |
| 52 | 1-cyclohexyl-6-(1-(3-methoxybenzoyl)azetidin-3-yl)-3-methyl-1H- | +++ |
| | pyrazolo[3,4-d]pyrimidin-4(5H)-one | |
| 53 | 1-cyclohexyl-6-(1-(3-methoxybenzoyl)piperidin-3-yl)-1H- | +++ |
| | pyrazolo[3,4-d]pyrimidin-4(5H)-one | |
| 54 | 1-cyclohexyl-6-(1-(2-(trifluoromethoxy)benzoyl)piperidin-3-yl)-1H- | +++ |
| | pyrazolo[3,4-d]pyrimidin-4(5H)-one | |
| 55 | N-(3-(3-(1-cyclohexyl-3-methyl-4-oxo-4,5-dihydro-1H-pyrazolo[3,4- | +++ |
| | d]pyrimidin-6-yl)piperidine-1-carbonyl)phenyl)butane-1-sulfonamide | |
| 56 | 1-cyclohexyl-6-(1-(4-methoxyphenylsulfonyl)piperidin-3-yl)-3- | +++ |
| | methyl-1H-pyrazolo[3,4-d]pyrimidin-4(5H)-one | |
| 57 | 1-cyclohexyl-6-(1-(2-methoxybenzoyl)piperidin-3-yl)-3-methyl-1H- | +++ |
| | pyrazolo[3,4-d]pyrimidin-4(5H)-one | |
| 58 | 1-cyclohexyl-6-(1-(2,2-difluorobenzo[d][1,3]dioxole-4- | +++ |
| | carbonyl)piperidin-3-yl)-3-methyl-1H-pyrazolo[3,4-d]pyrimidin- | |
| | 4(5H)-one | |
| 59 | 1-cyclohexyl-6-(1-(4-(dimethylamino)benzoyl)piperidin-3-yl)-3- | +++ |
| | methyl-1H-pyrazolo[3,4-d]pyrimidin-4(5H)-one | |
| 60 | 1-cyclohexyl-6-(1-(4-(dimethylamino)benzoyl)piperidin-3-yl)-1H- | +++ |
| | pyrazolo[3,4-d]pyrimidin-4(5H)-one | |
| 61 | 3-(1-cyclohexyl-4-oxo-4,5-dihydro-1H-pyrazolo[3,4-d]pyrimidin-6- | +++ |
| | yl)-N-(2-morpholinoethyl)azetidine-1-carboxamide | |
| 62 | N-(2-(1H-imidazol-4-yl)ethyl)-3-(1-cyclohexyl-4-oxo-4,5-dihydro- | +++ |
| | 1H-pyrazolo[3,4-d]pyrimidin-6-yl)azetidine-1-carboxamide | |
| 63 | 1-cyclohexyl-6-(1-(2,5-dimethoxyphenylsulfonyl)piperidin-3-yl)-1H- | +++ |
| | pyrazolo[3,4-d]pyrimidin-4(5H)-one | |
| 64 | N-(3-(3-(1-cyclohexyl-3-methyl-4-oxo-4,5-dihydro-1H-pyrazolo[3,4- | +++ |
| | d]pyrimidin-6-yl)piperidine-1-carbonyl)phenyl)methanesulfonamide | |
| 65 | 6-(1-(2-methoxybenzoyl)piperidin-3-yl)-1-phenyl-1H-pyrazolo[3,4- | +++ |
| | d]pyrimidin-4(5H)-one | |
| 66 | methyl 4-(3-(1-cyclohexyl-3-methyl-4-oxo-4,5-dihydro-1H- | +++ |
| | pyrazolo[3,4-d]pyrimidin-6-yl)piperidine-1-carbonyl)benzoate | |
| 67 | 6-(1-(4-(dimethylamino)benzoyl)piperidin-3-yl)-1-phenyl-1H- | +++ |
| | pyrazolo[3,4-d]pyrimidin-4(5H)-one | |
| 68 | 6-(1-(3-methoxybenzoyl)piperidin-3-yl)-1-phenyl-1H-pyrazolo[3,4- | +++ |
| | d]pyrimidin-4(5H)-one | |
| 69 | 3-(1-cyclohexyl-4-oxo-4,5-dihydro-1H-pyrazolo[3,4-d]pyrimidin-6- | +++ |
| | yl)-N-cyclopropylazetidine-1-carboxamide | |
| 70 | 1-cyclobutyl-6-(1-(4-methoxyphenylsulfonyl)piperidin-3-yl)-3- | +++ |
| | methyl-1H-pyrazolo[3,4-d]pyrimidin-4(5H)-one | |
| 71 | 1-cyclohexyl-6-(1-(3-methoxybenzoyl)piperidin-3-yl)-3-methyl-1H- | +++ |
| | pyrazolo[3,4-d]pyrimidin-4(5H)-one | |
| 72 | 6-(1-benzoylazetidin-3-yl)-1-cyclohexyl-3-methyl-1H-pyrazolo[3,4- | +++ |
| | d]pyrimidin-4(5H)-one | |
| 73 | methyl 3-(3-(1-cyclohexyl-3-methyl-4-oxo-4,5-dihydro-1H- | +++ |
| | pyrazolo[3,4-d]pyrimidin-6-yl)piperidine-1-carbonyl)benzaate | |
| 74 | 3-(1-cyclohexyl-4-oxo-4,5-dihydro-1H-pyrazolo[3,4-d]pyrimidin-6- | +++ |
| | yl)-N-((1-methyl-1H-imidazol-5-yl)methyl)azetidine-1-carboxamide | |
| 75 | 6-(1-(4-methoxyphenylsulfonyl)piperidin-3-yl)-3-methyl-1-phenyl- | +++ |
| | 1H-pyrazolo[3,4-d]pyrimidin-4(5H)-one | |
| 76 | 6-(1-(benzo[d][1,3]dioxole-5-carbonyl)piperidin-3-yl)-1-cyclohexyl- | +++ |
| | 1H-pyrazolo[3,4-d]pyrimidin-4(5H)-one | |
| 77 | 1-cyclohexyl-6-(1-(4-methoxybenzoyl)piperidin-3-yl)-3-methyl-1H- | +++ |
| | pyrazolo[3,4-d]pyrimidin-4(5H)-one | |
| 78 | 1-cyclobutyl-6-(1-(3-methoxybenzoyl)piperidin-3-yl)-3-methyl-1H- | ++ |
| | pyrazolo[3,4-d]pyrimidin-4(5H)-one | |
| 79 | 1-cyclohexyl-6-(1-(3-methoxyphenylsulfonyl)piperidin-3-yl)-1H- | ++ |
| | pyrazolo[3,4-d]pyrimidin-4(5H)-one | |
| 80 | 6-(1-(4-(dimethylamino)benzoyl)piperidin-3-yl)-3-methyl-1-phenyl- | ++ |
| | 1H-pyrazolo[3,4-d]pyrimidin-4(5H)-one | |
| 81 | 6-(1-(3-methoxybenzoyl)piperidin-3-yl)-3-methyl-1-phenyl-1H- | ++ |
| | pyrazolo[3,4-d]pyrimidin-4(5H)-one | |
| 82 | 1-cyclobutyl-6-(1-(2-methoxybenzoyl)piperidin-3-yl)-3-methyl-1H- | ++ |
| | pyrazolo[3,4-d]pyrimidin-4(5H)-one | |
| 83 | 1-cyclohexyl-6-(1-(piperidine-1-carbonyl)azetidin-3-yl)-1H- | ++ |
| | pyrazolo[3,4-d]pyrimidin-4(5H)-one | |
| 84 | 3-(1-cyclohexyl-4-oxo-4,5-dihydro-1H-pyrazolo[3,4-d]pyrimidin-6- | ++ |
| | yl)-N-(2-(pyrrolidin-1-yl)ethyl)azetidine-1-carboxamide | |
| 85 | 1-cyclohexyl-3-methyl-6-(1-(2-phenylacetoyl)azetidin-3-yl)-1H- | ++ |
| | pyrazolo[3,4-d]pyrimidin-4(5H)-one | |
| 86 | 1-cyclohexyl-3-methyl-6-(1-(p-tolylsulfonyl)piperidin-3-yl)-1H- | ++ |
| | pyrazolo[3,4-d]pyrimidin-4(5H)-one | |
| 87 | 1-cyclohexyl-3-methyl-6-(1-(2-methylbenzoyl)piperidin-3-yl)-1H- | ++ |
| | pyrazolo[3,4-d]pyrimidin-4(5H)-one | |
| 88 | 3-(1-cyclohexyl-4-oxo-4,5-dihydro-1H-pyrazolo[3,4-d]pyrimidin-6- | ++ |
| | yl)-N-(3-morpholinopropyl)azetidine-1-carboxamide | |
| 89 | 6-(1-benzoylpiperidin-3-yl)-1-cyclohexyl-3-methyl-1H-pyrazolo[3,4- | ++ |
| | d]pyrimidin-4(5H)-one | |
| 90 | 6-(1-(2-methoxybenzoyl)piperidin-3-yl)-3-methyl-1-phenyl-1H- | ++ |
| | pyrazolo[3,4-d]pyrimidin-4(5H)-one | |
| 91 | 6-(1-(2-chlorobenzoyl)piperidin-3-yl)-1-cyclohexyl-3-methyl-1H- | ++ |
| | pyrazolo[3,4-d]pyrimidin-4(5H)-one | |
| 92 | 1-cyclohexyl-6-(1-(2-fluorobenzoyl)piperidin-3-yl)-3-methyl-1H- | ++ |
| | pyrazolo[3,4-d]pyrimidin-4(5H)-one | |
| 93 | 1-cyclohexyl-6-(1-(2-methoxybenzoyl)azetidin-3-yl)-3-methyl-1H- | ++ |
| | pyrazolo[3,4-d]pyrimidin-4(5H)-one | |
| 94 | 1-cyclohexyl-6-(1-(morpholine-4-carbonyl)azetidin-3-yl)-1H- | ++ |
| | pyrazolo[3,4-d]pyrimidin-4(5H)-one | |
| 95 | 1-cyclohexyl-3-methyl-6-(1-(1-methyl-1H-pyrrole-2- | ++ |
| | carbonyl)piperidin-3-yl)-1H-pyrazolo[3,4-d]pyrimidin-4(5H)-one | |
| 96 | 1-cyclohexyl-3-methyl-6-(1-(4-(trifluoromethyl)benzoyl)piperidin-3- | ++ |
| | yl)-1H-pyrazolo[3,4-d]pyrimidin-4(5H)-one | |
| 97 | 1-cyclohexyl-3-methyl-6-(1-(thiophene-3-carbonyl)piperidin-3-yl)- | + |
| | 1H-pyrazolo[3,4-d]pyrimidin-4(5H)-one | |
| 98 | 1-cyclohexyl-3-methyl-6-(1-(1-methyl-1H-imidazol-4- | + |
| | ylsulfonyl)piperidin-3-yl)-1H-pyrazolo[3,4-d]pyrimidin-4(5H)-one | |
| 99 | 3-(1-cyclohexyl-3-methyl-4-oxo-4,5-dihydro-1H-pyrazolo[3,4- | + |
| | d]pyrimidin-6-yl)-N-(2-methoxyphenyl)piperidine-1-carboxamide | |
| 100 | 6-(1-(2-chlorophenylsulfonyl)piperidin-3-yl)-1-cyclohexyl-3-methyl- | + |
| | 1H-pyrazolo[3,4-d]pyrimidin-4(5H)-one | |
| 101 | 1-cyclohexyl-3-methyl-6-(1-(thiophene-2-carbonyl)piperidin-3-yl)- | + |
| | 1H-pyrazolo[3,4-d]pyrimidin-4(5H)-one | |
| 102 | 4-(3-(1-cyclobutyl-3-methyl-4-oxo-4,5-dihydro-1H-pyrazolo[3,4- | + |
| | d]pyrimidin-6-yl)piperidine-1-carbonyl)benzonitrile | |
| 103 | 6-(1-(benzylsulfonyl)piperidin-3-yl)-1-cyclohexyl-3-methyl-1H- | + |
| | pyrazolo[3,4-d]pyrimidin-4(5H)-one | |
| 104 | 1-cyclohexyl-6-(1-(pentylsulfonyl)azetidin-3-yl)-1H-pyrazolo[3,4- | + |
| | d]pyrimidin-4(5H)-one | |
| 105 | 1-cyclohexyl-6-(1-(4-(trifluoromethoxy)phenylsulfonyl)piperidin-3- | + |
| | yl)-1H-pyrazolo[3,4-d]pyrimidin-4(5H)-one | |
| 106 | 1-cyclohexyl-3-methyl-6-(1-(phenylsulfonyl)piperidin-3-yl)-1H- | + |
| | pyrazolo[3,4-d]pyrimidin-4(5H)-one | |
| 107 | 1-cyclohexyl-3-methyl-6-(1-(2- | + |
| | (trifluoromethoxy)phenylsulfonyl)piperidin-3-yl)-1H-pyrazolo[3,4- | |
| | d]pyrimidin-4(5H)-one | |
| 108 | N-(4-fluorophenyl)-3-(1-(1-isopropylazetidin-3-yl)-4-oxo-4,5-dihydro- | + |
| | 1H-pyrazolo[3,4-d]pyrimidin-6-yl)azetidine-1-carboxamide | |
| 109 | 4-(3-(3-methyl-4-oxo-1-phenyl-4,5-dihydro-1H-pyrazolo[3,4- | + |
| | d]pyrimidin-6-yl)piperidine-1-carbonyl)benzonitrile | |
| 110 | 4-(3-(4-oxo-1-phenyl-4,5-dihydro-1H-pyrazolo[3,4-d]pyrimidin-6- | + |
| | yl)piperidine-1-carbonyl)benzonitrile | |
| 111 | 6-(1-(4-methoxyphenylsulfonyl)piperidin-3-yl)-3-methyl-1-propyl-1H- | + |
| | pyrazolo[3,4-d]pyrimidin-4(5H)-one | |
| 112 | 1-cyclohexyl-3-methyl-6-(1-(3-phenylpropanoyl)piperidin-3-yl)-1H- | + |
| | pyrazolo[3,4-d]pyrimidin-4(5H)-one | |
| 113 | N-(4-(3-(1-cyclohexyl-3-methyl-4-oxo-4,5-dihydro-1H-pyrazolo[3,4- | + |
| | d]pyrimidin-6-yl)piperidine-1-carbonyl)phenyl)acetamide | |
| 114 | 3-(1-cyclohexyl-3-ethyl-4-oxo-4,5-dihydro-1H-pyrazolo[3,4- | ++ |
| | d]pyrimidin-6-yl)-N-(2-(difluoromethoxy)phenyl)azetidine-1- | |
| | carboxamide | |
| 115 | benzyl 3-(1-cyclohexyl-3-methyl-4-oxo-4,5-dihydro-1H-pyrazolo[3,4- | +++ |
| | d]pyrimidin-6-yl)azetidine-1-carboxylate | |
| 116 | 4-(4-isopropylpiperazin-1-yl)phenyl 3-(1-tert-butyl-4-oxo-4,5-dihydro- | ++++ |
| | 1H-pyrazolo[3,4-d]pyrimidin-6-yl)azetidine-1-carboxylate | |
| 117 | cyclohexylmethyl 3-(1-tert-butyl-4-oxo-4,5-dihydro-1H-pyrazolo[3,4- | ++++ |
| | d]pyrimidin-6-yl)azetidine-1-carboxylate | |
| 118 | 3-chlorophenyl 3-(1-tert-butyl-4-oxo-4,5-dihydro-1H-pyrazolo[3,4- | ++++ |
| | d]pyrimidin-6-yl)azetidine-1-carboxylate | |
| 119 | benzo[d][1,3]dioxol-5-yl 3-(1-tert-butyl-4-oxo-4,5-dihydro-1H- | ++++ |
| | pyrazolo[3,4-d]pyrimidin-6-yl)azetidine-1-carboxylate | |
| 120 | cyclohexylmethyl 3-(1-cyclohexyl-4-oxo-4,5-dihydro-1H- | ++++ |
| | pyrazolo[3,4-d]pyrimidin-6-yl)azetidine-1-carboxylate | |
| 121 | 4-fluorophenyl 3-(1-cyclohexyl-4-oxo-4,5-dihydro-1H-pyrazolo[3,4- | ++++ |
| | d]pyrimidin-6-yl)azetidine-1-carboxylate | |
| 122 | neopentyl 3-(1-cyclohexyl-4-oxo-4,5-dihydro-1H-pyrazolo[3,4- | +++ |
| | d]pyrimidin-6-yl)azetidine-1-carboxylate | |
| 123 | tert-butyl 3-(1-cyclohexyl-3-methyl-4-oxo-4,5-dihydro-1H- | +++ |
| | pyrazolo[3,4-d]pyrimidin-6-yl)azetidine-1-carboxylate | |
| 124 | phenyl 3-(1-cyclohexyl-3-methyl-4-oxo-4,5-dihydro-1H-pyrazolo[3,4- | ++ |
| | d]pyrimidin-6-yl)piperidine-1-carboxylate | |
| 125 | 6-(1-(1H-benzo[d]imidazol-2-yl)azetidin-3-yl)-1-cyclohexyl-1H- | ++++ |
| | pyrazolo[3,4-d]pyrimidin-4(5H)-one | |
| 126 | 6-(1-(6-bromo-1H-benzo[d]imidazol-2-yl)azetidin-3-yl)-1-tert-butyl- | ++++ |
| | 1H-pyrazolo[3,4-d]pyrimidin-4(5H)-one | |
| 127 | 1-tert-butyl-6-(1-(5-(trifluoromethyl)-1H-benzo[d]imidazol-2- | ++++ |
| | yl)azetidin-3-yl)-1H-pyrazolo[3,4-d]pyrimidin-4(5H)-one | |
| 128 | 6-(1-(1H-benzo[d]imidazol-2-yl)azetidin-3-yl)-1-tert-butyl-1H- | ++++ |
| | pyrazolo[3,4-d]pyrimidin-4(5H)-one | |
| 129 | 6-(1-(benzo[d]thiazol-2-yl)azetidin-3-yl)-1-cyclohexyl-1H- | +++ |
| | pyrazolo[3,4-d]pyrimidin-4(5H)-one | |
| 130 | 6-(1-(benzo[d]oxazol-2-yl)azetidin-3-yl)-1-cyclohexyl-1H- | +++ |
| | pyrazolo[3,4-d]pyrimidin-4(5H)-one | |
| 131 | 1-cyclohexyl-6-(1-(1-methyl-1H-benzo[d]imidazol-2-yl)azetidin-3-yl)- | ++ |
| | 1H-pyrazolo[3,4-d]pyrimidin-4(5H)-one | |
| 132 | 1-cyclohexyl-6-(1-(4-phenyloxazol-2-yl)azetidin-3-yl)-1H- | ++++ |
| | pyrazolo[3,4-d]pyrimidin-4(5H)-one | |
| 133 | 1-cyclohexyl-6-(1-(5-phenyl-1,3,4-oxadiazol-2-yl)azetidin-3-yl)-1H- | +++ |
| | pyrazolo[3,4-d]pyrimidin-4(5H)-one | |
| 134 | 1-cyclohexyl-6-(1-(5-(morpholinomethyl)thiazol-2-yl)azetidin-3-yl)- | +++ |
| | 1H-pyrazolo[3,4-d]pyrimidin-4(5H)-one | |
| 135 | 1-cyclohexyl-6-(1-(5-(piperidin-1-ylmethyl)thiazol-2-yl)azetidin-3-yl)- | +++ |
| | 1H-pyrazolo[3,4-d]pyrimidin-4(5H)-one | |
| 136 | 1-cyclohexyl-6-(1-(5-phenyloxazol-2-yl)azetidin-3-yl)-1H- | +++ |
| | pyrazolo[3,4-d]pyrimidin-4(5H)-one | |
| 137 | 1-cyclohexyl-6-(1-(5-((4-methylpiperazin-1-yl)methyl)thiazol-2- | +++ |
| | yl)azetidin-3-yl)-1H-pyrazolo[3,4-d]pyrimidin-4(5H)-one | |
| 138 | 6-(1-(5-(azepan-1-ylmethyl)thiazol-2-yl)azetidin-3-yl)-1-cyclohexyl- | +++ |
| | 1H-pyrazolo[3,4-d]pyrimidin-4(5H)-one | |
| 139 | 1-tert-butyl-3-methyl-6-(1-(1-phenyl-1H-tetrazol-5-yl)azetidin-3-yl)- | +++ |
| | 1H-pyrazolo[3,4-d]pyrimidin-4(5H)-one | |
| 140 | 6-(1-(5-bromothiazol-2-yl)azetidin-3-yl)-1-cyclohexyl-1H- | ++ |
| | pyrazolo[3,4-d]pyrimidin-4(5H)-one | |
| 141 | 1-tert-butyl-6-(1-(thiazol-2-yl)azetidin-3-yl)-1H-pyrazolo[3,4- | ++ |
| | d]pyrimidin-4(5H)-one | |
| 142 | 1-cyclohexyl-6-(1-(4-(morpholinomethyl)thiazol-2-yl)azetidin-3-yl)- | +++ |
| | 1H-pyrazolo[3,4-d]pyrimidin-4(5H)-one | |
| 143 | 1-cyclohexyl-6-(1-(4-((4-isopropylpiperazin-1-yl)methyl)thiazol-2- | + |
| | yl)azetidin-3-yl)-1H-pyrazolo[3,4-d]pyrimidin-4(5H)-one | |
| 144 | 1-tert-butyl-6-(1-(5-(4-methylpiperazin-1-yl)-1H-benzo[d]imidazol-2- | +++ |
| | yl)azetidin-3-yl)-1H-pyrazolo[3,4-d]pyrimidin-4(5H)-one | |
| 145 | 1-cyclohexyl-6-(1-(4-(morpholinosulfonyl)phenyl)azetidin-3-yl)-1H- | ++++ |
| | pyrazolo[3,4-d]pyrimidin-4(5H)-one | |
| 146 | 6-(1-(3-amino-4-nitrophenyl)azetidin-3-yl)-1-cyclohexyl-1H- | ++++ |
| | pyrazolo[3,4-d]pyrimidin-4(5H)-one | |
| 147 | 6-(1-(4-acetoylphenyl)azetidin-3-yl)-1-cyclohexyl-1H-pyrazolo[3,4- | ++++ |
| | d]pyrimidin-4(5H)-one | |
| 148 | 4-(3-(1-cyclohexyl-4-oxo-4,5-dihydro-1H-pyrazolo[3,4-d]pyrimidin-6- | ++++ |
| | yl)azetidin-1-yl)benzonitrile | |
| 149 | 1-cyclohexyl-6-(1-(3-methyl-4-nitrophenyl)azetidin-3-yl)-1H- | ++++ |
| | pyrazolo[3,4-d]pyrimidin-4(5H)-one | |
| 150 | 2-(3-(1-cyclohexyl-4-oxo-4,5-dihydro-1H-pyrazolo[3,4-d]pyrimidin-6- | +++ |
| | yl)azetidin-1-yl)benzonitrile | |
| 151 | 6-(1-(2-acetoylphenyl)azetidin-3-yl)-1-cyclohexyl-1H-pyrazolo[3,4- | +++ |
| | d]pyrimidin-4(5H)-one | |
| 152 | 1-cyclohexyl-6-(1-(4-(trifluoromethyl)phenyl)azetidin-3-yl)-1H- | +++ |
| | pyrazolo[3,4-d]pyrimidin-4(5H)-one | |
| 153 | 1-cyclohexyl-6-(1-(5-nitropyridin-2-yl)piperidin-3-yl)-1H- | + |
| | pyrazolo[3,4-d]pyrimidin-4(5H)-one | |
| 154 | 1-cyclohexyl-6-(1-(5-(trifluoromethyl)pyridin-2-yl)piperidin-3-yl)-1H- | + |
| | pyrazolo[3,4-d]pyrimidin-4(5H)-one | |
| 155 | 1-cyclohexyl-6-(1-(4-(morpholinomethyl)phenyl)azetidin-3-yl)-1H- | ++++ |
| | pyrazolo[3,4-d]pyrimidin-4(5H)-one | |
| 156 | 1-cyclohexyl-6-(1-(2-(morpholinomethyl)phenyl)azetidin-3-yl)-1H- | +++ |
| | pyrazolo[3,4-d]pyrimidin-4(5H)-one | |
| 157 | 6-(1-(4-tert-butylthiazol-2-yl)azetidin-3-yl)-1-cyclohexyl-1H- | +++ |
| | pyrazolo[3,4-d]pyrimidin-4(5H)-one | |
| 158 | 1-cyclohexyl-6-(1-(5-phenyl-4H-1,2,4-triazol-3-yl)azetidin-3-yl)-1H- | ++++ |
| | pyrazolo[3,4-d]pyrimidin-4(5H)-one | |
| 159 | 1-cyclohexyl-6-(1-(4-phenylthiazol-2-yl)azetidin-3-yl)-1H- | ++++ |
| | pyrazolo[3,4-d]pyrimidin-4(5H)-one | |
| 160 | 1-cyclohexyl-6-(1-(5-phenylthiazol-2-yl)azetidin-3-yl)-1H- | +++ |
| | pyrazolo[3,4-d]pyrimidin-4(5H)-one | |
| 161 | 1-cyclohexyl-6-(1-(4-(4-(4-isopropylpiperazin-1-yl)phenyl)thiazol-2- | ++++ |
| | yl)azetidin-3-yl)-1H-pyrazolo[3,4-d]pyrimidin-4(5H)-one | |
| 162 | 1-cyclohexyl-6-(1-(5-(morpholinomethyl)-4-phenylthiazol-2- | ++++ |
| | yl)azetidin-3-yl)-1H-pyrazolo[3,4-d]pyrimidin-4(5H)-one | |
| 163 | 1-tert-butyl-6-(1-(pyridin-2-yl)azetidin-3-yl)-1H-pyrazolo[3,4- | +++ |
| | d]pyrimidin-4(5H)-one | |
| 164 | 1-tert-butyl-6-(1-(4-(morpholinomethyl)phenyl)azetidin-3-yl)-1H- | ++++ |
| | pyrazolo[3,4-d]pyrimidin-4(5H)-one | |
| 165 | 1-cyclohexyl-6-(1-(4-methoxyphenyl)azetidin-3-yl)-1H-pyrazolo[3,4- | +++ |
| | d]pyrimidin-4(5H)-one | |
| 166 | 1-cyclohexyl-6-(1-(3-methoxyphenyl)azetidin-3-yl)-1H-pyrazolo[3,4- | +++ |
| | d]pyrimidin-4(5H)-one | |
| 167 | N-(4-(3-(1-cyclohexyl-4-oxo-4,5-dihydro-1H-pyrazolo[3,4- | +++ |
| | d]pyrimidin-6-yl)azetidin-1-yl)phenyl)acetamide | |
| 168 | 1-cyclohexyl-6-(1-phenylazetidin-3-yl)-1H-pyrazolo[3,4-d]pyrimidin- | +++ |
| | 4(5H)-one | |
| 169 | 1-cyclohexyl-6-(1-(2-methoxyphenyl)azetidin-3-yl)-1H-pyrazolo[3,4- | +++ |
| | d]pyrimidin-4(5H)-one | |
| 170 | 3-(1-cyclohexyl-3-methyl-4-oxo-4,5-dihydro-1H-pyrazolo[3,4- | +++ |
| | d]pyrimidin-6-yl)-N-(4-fluorobenzyl)-N-methylazetidine-1- | |
| | carboxamide | |
| 171 | 1-tert-butyl-6-(1-((1-methyl-1H-benzo[d]imidazol-2- | ++ |
| | yl)methyl)azetidin-3-yl)-1H-pyrazolo[3,4-d]pyrimidin-4(5H)-one | |
| 172 | 6-(1-(benzo[d]thiazol-2-ylmethyl)azetidin-3-yl)-1-cyclohexyl-1H- | ++ |
| | pyrazolo[3,4-d]pyrimidin-4(5H)-one | |
| 173 | 2-(3-(1-tert-butyl-4-oxo-4,5-dihydro-1H-pyrazolo[3,4-d]pyrimidin-6- | +++ |
| | yl)azetidin-1-yl)-N-phenylacetamide | |
| 174 | 1-tert-butyl-6-(1-(2-oxo-2-phenylethyl)azetidin-3-yl)-1H- | +++ |
| | pyrazolo[3,4-d]pyrimidin-4(5H)-one | |
| 175 | 6-(1-((1H-benzo[d]imidazol-2-yl)methyl)azetidin-3-yl)-1-tert-butyl- | + |
| | 1H-pyrazolo[3,4-d]pyrimidin-4(5H)-one | |
| 176 | 6-(1-benzylpiperidin-3-yl)-1-cyclohexyl-1H-pyrazolo[3,4- | + |
| | d]pyrimidin-4(5H)-one | |
| 177 | (Z)-N′-cyano-3-(1-cyclohexyl-4-oxo-4,5-dihydro-1H-pyrazolo[3,4- | ++++ |
| | d]pyrimidin-6-yl)-N-(4-fluorophenyl)azetidine-1-carboximidamide | |
| 178 | (Z)-N′-cyano-3-(1-cyclohexyl-4-oxo-4,5-dihydro-1H-pyrazolo[3,4- | ++++ |
| | d]pyrimidin-6-yl)-N-(cyclohexylmethyl)azetidine-1-carboximidamide | |
| 179 | (Z)-3-(1-tert-butyl-4-oxo-4,5-dihydro-1H-pyrazolo[3,4-d]pyrimidin-6- | ++++ |
| | yl)-N′-cyano-N-(4-fluorophenyl)azetidine-1-carboximidamide | |
| 180 | (Z)-1-cyclohexyl-6-(1-(1-(cyclohexylmethylamino)-2- | +++ |
| | nitrovinyl)azetidin-3-yl)-1H-pyrazolo[3,4-d]pyrimidin-4(5H)-one | |
| 181 | N-(2-(3-(1-cyclohexyl-3-methyl-4-oxo-4,5-dihydro-1H-pyrazolo[3,4- | ++ |
| | d]pyrimidin-6-yl)piperidine-1-carbonyl)phenyl)methanesulfonamide | |
| 182 | 3-(1-cyclohexyl-4-oxo-4,5-dihydro-1H-pyrazolo[3,4-d]pyrimidin-6- | ++ |
| | yl)-N-(piperidin-3-yl)azetidine-1-carboxamide 2,2,2-trifluoroacetate | |
| 183 | 3-(1-cyclohexyl-4-oxo-4,5-dihydro-1H-pyrazolo[3,4-d]pyrimidin-6- | +++ |
| | yl)-N-(1-isopropylpiperidin-3-yl)azetidine-1-carboxamide | |
| 184 | 3-(1-cyclohexyl-4-oxo-4,5-dihydro-1H-pyrazolo[3,4-d]pyrimidin-6- | ++ |
| | yl)-N-((1-isopropylpiperidin-4-yl)methyl)azetidine-1-carboxamide | |
| 185 | 3-[1-(1-Isopropyl-azetidin-3-yl)-4-oxo-4,5-dihydro-1H-pyrazolo[3,4- | + |
| | d]pyrimidin-6-yl]-azetidine-1-carboxylic acid (4-fluoro-phenyl)-amide | |

### 2.2 PDE Selectivity Panel

In one aspect the compounds of the invention are more potent against PDE1A than against other PDE isoforms. In a particular embodiment, the compounds are 2 fold more potent against PDE1A than against one or more of the other isoforms. In an alternative embodiment, the compounds of the invention are 5 fold, particularly 10-fold, particularly 20-fold more potent against PDE1A than against one or more of the other isoforms of PDE. In particular, the compounds of the invention are more potent against PDE1A than against at least one of PDE1B, PDE2A, PDE4A or PDE5A. In particular the compounds are more potent against PDE1A than against at least two of PDE1B, PDE2A, PDE4A or PDE5A. In particular the compounds are more potent against PDE1A than against all of the other PDE isoforms. Methods for testing the selectivity of the compounds against a range of PDE isoforms will be familiar to those of skill in the art, and for example, may measure comparative IC₅₀ values or percentage inhibition values at a set concentration. Typical methods are described below.

To test the selectivity of the compounds against a panel of PDE'S, lysate derived from transiently transfected HEK293 cells (transfected with PDE5A, PDE1B, PDE2A or PDE4A for 48 h) is used as the enzyme source.

The dose response of compounds on PDE5A lysate is performed using the cGMP bulk htrf kit from Cisbio (catnr 62GM2PEC). The principle of this kit is based on the HTRF® technology (Homogeneous Time-Resolved Fluorescence). The method is based on the competition between native cGMP and the cGMP labeled with d2. The tracer binding is visualized by a monoclonal antibody against cGMP, labeled with Cryptate. The specific signal (i.e. energy transfer) is inversely proportional to the concentration of cGMP in the sample. PDE5A hydrolyses cGMP into 5′GMP; the decrease in cGMP concentration upon PDE5A activity will result in an increased signal. A PDE5A inhibitor will cause a decrease of this signal.

For the enzymatic reaction, a mix of 20 μL with PDE5A lysate, 400 nM cGMP, and the compound is made in a black 384-plate. The reaction buffer consists of Tris 20 mM pH 7.4, 3 mM MgCl₂, 1.5 mM CaCl₂, 0.2 mg/mL BSA and 0.001% Brij-35. After an incubation of 25 minutes at room temperature, the reaction is stopped by the addition of 10 μL labeled cGMP-d2 and 10 μL anti-cGMP-Cryptate. After 1 hour incubation at room temperature, the readout is performed on the Envision (excitation 360 nm; emission donor 615 nm; emission acceptor 665 nm).

All compounds are tested in duplicate starting from 20 μM and 20 nM followed by a 1/3 serial dilution, 8 points (20 μM-6.67 μM-2.22 μM-740 nM-247 nM-82 nM-27 nM-9 nM and 20 nM-6.67 nM-2.22 nM-740 pM-247 pM-82 pM-27 pM-9 pM) in a final concentration of 1% DMSO. As positive control the compound Vardenafil is also added in dose response. For the calculation of z′ and PIN values 1% DMSO is used as positive control (100% inhibition) and lysate in 1% DMSO as negative control (0% inhibition).

For the single dose screening on PDE1B, PDE2A and PDE4A lysates an assay using the cAMP dynamic 2 bulk htrf kit from Cisbio (catnr 62AM4PEC) is used. The principle of this kit is based on the HTRF® technology (Homogeneous Time-Resolved Fluorescence). The method is based on the competition between native cAMP and the cAMP labeled with d2. The tracer binding is visualized by a monoclonal antibody against cAMP, labeled with Cryptate. The specific signal (i.e. energy transfer) is inversely proportional to the concentration of cAMP in the sample.

For the enzymatic reaction, a mixture is made of 10 μL with PDE1B, PDE2A or PDE4A lysate, 100 nM cAMP and the compound (50 nM in a final concentration of 1% DMSO) in a black 384-plate. The reaction buffer is Tris 20 mM pH 7.4, 37.5 U/ml calmodulin, 3 mM MgCl₂, 1.5 mM CaCl₂, 0.2 mg/mL BSA and 0.001% Brij-350. After an incubation of 25 minutes at room temperature, the reaction is stopped by the addition of 5 μL labelled cAMP-d2 and 5 μL anti-cAMP-Cryptate. After 1 hour incubation at room temperature, the readout is performed on the Envision (excitation 360 nm; emission donor 615 nm; emission acceptor 665 nm).

PDE1B, PDE2A and PDE4A hydrolyse cAMP into 5′AMP; this decrease in cAMP concentration will result in an increase in signal. A PDE1A, PDE2A or PDE4A inhibitor will result in a decrease of this signal.

1% DMSO (100% inhibition) may be used as a positive control, lysate with 1% DMSO (0% inhibition) may be used as a negative control. The positive and negative control are used to calculate z′ and PIN values.

All compounds may be screened at a single concentration of 50 nM. The hit criteria is set at PIN 50 (50% inhibition).

It should be understood that factors such as the differential cell penetration capacity of the various compounds can contribute to discrepancies between the activity of the compounds in the in vitro biochemical and cellular assays.

It will be appreciated by those skilled in the art that the foregoing description is exemplary and explanatory in nature, and is intended to illustrate the invention and its preferred embodiments. Through routine experimentation, an artisan will recognise apparent modifications and variations that may be made without departing from the spirit of the invention. Thus, the invention is intended to be defined not by the above description, but by the following claims and their equivalents.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All such modifications coming within the scope of the appended claims are intended to be included therein.

The chemical names of compounds given in this application were generated using various commercially available chemical naming software tools including MDL's ISIS Draw Autonom Software tool, and were not verified. Preferably, in the event of inconsistency, the depicted structure governs.

### REFERENCES

Andreakos E, et al. (2003). Arthritis Rheum. 48: 1901-12.
Choy E H, Panayi G S. (2001). N Engl J. Med. 344: 907-16.
Clegg D O et al. (2006) N Engl J. Med. 2006 354:795-808. Glucosamine, chondroitin sulfate, and the two in combination for painful knee osteoarthritis.
Coussens L M, et al. (2002). Science 295: 2387-92.
Creemers E E, et al. (2001). Circ Res. 2001 89:201-10
Cunnane G, et al. (2001). Arthritis Rheum 44: 2263-74.
Edwards J. C. W., Szczepanski L., Szechinski J., Filipowicz-Sosnowska A., Emery P., Close D. R.,
Stevens R. M., Shaw T. (2004) N Engl J. Med. 350:2572-2581.
EMBO J. 23:4780-91.
Firestein G S. (2003). Nature. 423:356-61.
Gapski R, et al. (2004). J Periodontol. 75:441-52.
Geng Y, Zhou L, Thompson W J, Lotz M. (1998) Cyclic GMP and cGMP-binding phosphodiesterase are required for interleukin-1-induced nitric oxide synthesis in human articular chondrocytes. J Biol. Chem. 273:27484-91.
Gomez-Reino J J, et al. (2003). Arthritis Rheum. 48: 2122-7.
Goraya T A, Cooper D M. (2005) Ca2+-calmodulin-dependent phosphodiesterase (PDE1): current perspectives. Cell Signal. 17:789-97.
Harder K. W. et al. Biochem. J. 1994 398 395
Kakkar R, Raju R V, Sharma R K. (1999) Calmodulin-dependent cyclic nucleotide phosphodiesterase (PDE1). Cell Mol Life Sci. 55:1164-86.
Kremer J. M., Westhovens R., Leon M., Di Giorgio E., Alten R., Steinfeld S., Russell A., Dougados M., Emery P., Nuamah I. F., Williams G. R., Becker J.-C., Hagerty D. T., Moreland L. W. (2003) N Engl J. Med. 349:1907-1915.
Lee D M, Weinblatt M E (2001). Lancet. 358: 903-11.
Lugnier C. (2006) Pharmacol Ther. 2006 109:366-98. Review. Cyclic nucleotide phosphodiesterase (PDE) superfamily: a new target for the development of specific therapeutic agents.
Murray F et al. (2006) Am J Physiol Lung Cell Mol. Physiol. Expression and Activity of cAMP Phosphodiesterase Isoforms in Pulmonary Artery Smooth Muscle Cells from Patients with Pulmonary Hypertension: Role for PDE1. In publication.
New L, Jiang Y, Han J. (2003) Regulation of PRAK subcellular location by p38 MAP kinases. Mol Biol Cell. 14(6):2603-16.
O'Dell J R. Leff R, Paulsen G, Haire C, Mallek J, Eckhoff P J, Fernandez A, Blakely K, Wees S, Stoner J, Hadley S, Felt J, Palmer W, Waytz P, Churchill M, Klassen L, Moore G. (2002) Arthritis Rheum. 46:1164-70.
O'Dell J R. (2004) Therapeutic strategies for rheumatoid arthritis. N Engl J. Med. 350(25):2591-602.
Reed T M et al. (2002) Phosphodiesterase 1B knock-out mice exhibit exaggerated locomotor hyperactivity and DARPP-32 phosphorylation in response to dopamine agonists and display impaired spatial learning. J. Neurosci. 22:5188-97.
Reif S, Somech R, Brazovski E, Reich R, Belson A, Konikoff F M, Kessler A. (2005) Digestion. 71:124-130.
Rosenberg G A. (2002). Glia. 39:279-91.
S. A. Beers et al. Bioorg. Med. Chem. 1997 5 2203
Schanstra J P, et al. (2002). J Clin Invest. 110:371-9.
Seternes O M, Mikalsen T, Johansen B, Michaelsen E, Armstrong C G, Morrice N A, Turgeon B, Meloche S, Moens U, Keyse S M. (2004) Activation of MK5/PRAK by the atypical MAP kinase ERK3 defines a novel signal transduction pathway.
Shi Y, Kotlyarov A, Laabeta K, Gruber A D, Butt E, Marcus K, Meyer H E, Friedrich A, Volk H D, Gaestel M. (2003) Elimination of protein kinase MK5/PRAK activity by targeted homologous recombination. Mol Cell Biol. 23:7732-41.
Smolen J S, Steiner G. (2003). Nat Rev Drug Discov. 2: 473-88.
St Clair E W, van der Heijde D M, Smolen J S, Maini R N, Bathon J M, Emery P, Keystone E, Schiff M, Kalden J R, Wang B, Dewoody K, Weiss R, Baker D; (2004) Combination of infliximab and methotrexate therapy for early rheumatoid arthritis: a randomized, controlled trial. Arthritis Rheum. 50:3432-43.
Suzuki R, et al. (2004). Treat Respir Med. 3:17-27.
Tenor H, Hedbom E, Hauselmann H J, Schudt C, Hatzelmann A. Phosphodiesterase isoenzyme families in human osteoarthritis chondrocytes—functional importance of phosphodiesterase 4. Br J. Pharmacol. (2002) 135:609-18.
Wieland H A, Michaelis M, Kirschbaum B J, Rudolphi K A. (2005). Nat Rev Drug Discov. 4:331-44. Osteoarthritis—an untreatable disease?
Zhang K Y et al. (2004) A glutamine switch mechanism for nucleotide selectivity by phosphodiesterases. Mol. Cell. 2004 15:279-86.

1. A compound according to formula Ia, Ib, Ic, Id, Ie, If, Ig, or Ih: 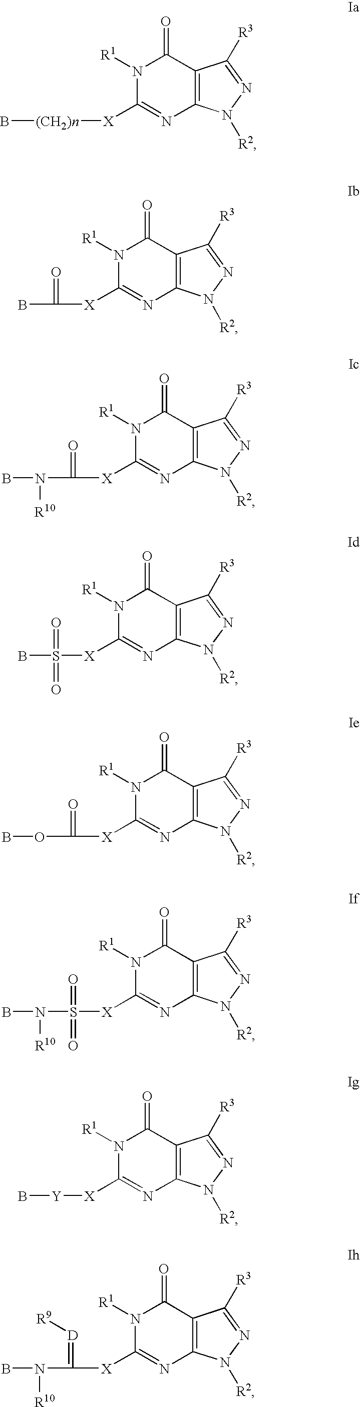
wherein:
X represents a carbon-carbon bonded nitrogen-containing heterocycloalkyl group;
B represents substituted or unsubstituted C₁-C₆ alkyl, or C₁-C₆ haloalkyl;
or B represents substituted or unsubstituted cycloalkyl, heterocycloalkyl, cycloalkylalkyl or heterocycloalkylalkyl;
or B represents substituted or unsubstituted aralkyl, aryl or heteroaryl;
or with respect to a compound according to the formulae Ia or Ig, B further includes H, NO₂, C₁-C₆ alkyl, halo, —CO-aryl, —CO-heteroaryl, —CO—N(R¹⁰)-aryl, or CO—N(R¹⁰)-heteroaryl;
Y represents a bond, substituted or unsubstituted aryl or substituted or unsubstituted heteroaryl;
the group B—(CH₂)n-, B—CO—, B—N(R¹⁰)CO—, B—SO₂—, B—OCO—, B—N(R¹⁰)SO₂—, B—Y— or B—NR¹⁰-D(R⁹)— is linked to X via a nitrogen atom within the X group;
D represents CH or N, with the proviso that when D represents CH, R⁹ represents —NO₂ and when D represents N, R⁹ represents CN;
R¹ represents H, C₁-C₆ alkyl, (CH₂)n-aryl, cycloalkyl or a —C₁-C₆ alkyl-cycloalkyl group, each of which may optionally be substituted with one or more groups selected from halogen, CN, CF₃, —NR⁴R⁵, —NR⁵COR⁴, —CONR⁴R⁵, —NR⁵SO₂R⁴, —SO₂NR⁵R⁴, C₁-C₆ alkyl, C₁-C₆ alkoxy, —COR⁴, —CO₂R⁴, or —SO₂R⁴;
R² represents H, C₁-C₆ alkyl, cycloalkyl, heterocycloalkyl, cycloalkylalkyl, (CH₂)n-aryl, or a heteroaryl group, each of which may optionally be substituted with one or more groups selected from halogen, CN, —NR⁴R⁵, —NR⁵COR⁴, —CONR⁴R⁵, —NR⁵SO₂R⁴, —SO₂NR⁵R⁴, C₁-C₆ alkyl, —C₁-C₆ haloalkyl, C₁-C₆ alkoxy, —C₁-C₆ haloalkoxy, —COR⁴, —CO₂R⁴, or SO₂R⁴;
R³ represents H, halogen, C₁-C₆ alkyl, cycloalkyl, (CH₂)n-aryl, aryl, or a heteroaryl group, each of which may optionally be substituted with one or more groups selected from halogen, CN, —NR⁴R⁵, —NR⁵COR⁴, —CONR⁴R⁵, —NR⁵SO₂R⁴, —SO₂NR⁵R⁴, C₁-C₆ alkyl, C₁-C₆ haloalkyl, C₁-C₆ alkoxy, C₁-C₆ haloalkoxy, COR⁴, CO₂R⁴, or SO₂R⁴;
R⁴ represents H, C₁-C₆ alkyl, C₁-C₆ haloalkyl, cycloalkyl, or heterocycloalkyl;
R⁵ represents H, C₁-C₆ alkyl, or cycloalkyl;
R⁹ represents CN or NO₂;
R¹⁰ represents H or C₁-C₆ alkyl; and
each “n” independently represents 0, 1, 2 or 3;
or a pharmaceutically acceptable salt thereof or isotopic variants thereof, stereoisomers or tautomers thereof.

2. A compound according to claim 1 wherein X is selected from piperidine, pyrrolidine and azetidine.

3. A compound according to claim 1 wherein the compound is according to formulae IIa, IIb, IIc, IId, IIe, IIf, IIg, or IIh: 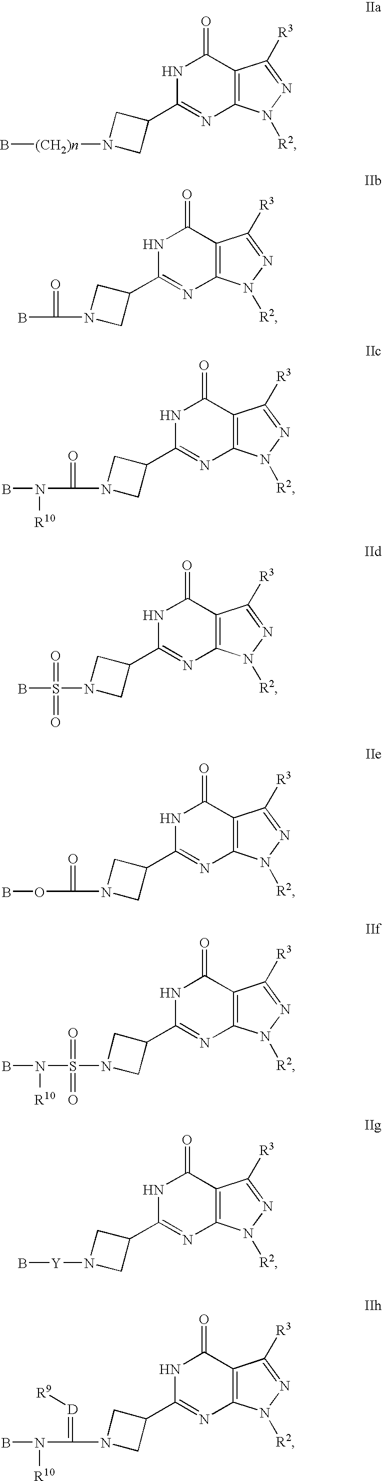
wherein B, Y, D, R², R³ and R⁹ are as in claim 1; R¹⁰ is H or Me; or a pharmaceutically acceptable salt, hydrate, solvate or prodrug thereof or isotopic variants thereof, stereoisomers or tautomers thereof.

4. (canceled)

5. A compound according to claim 1 wherein R² is C₁-C₆ alkyl, cycloalkyl, aryl, heteroaryl or heterocycloalkyl.

6. A compound according to claim 1 wherein R² is Me, i-Pr, t-Bu, cyclohexyl, cyclopentyl, cyclobutyl, phenyl, 4-fluorophenyl, pyridyl or pyrrolidinyl.

7. A compound according to claim 1 wherein R³ is H or C₁-C₆ alkyl.

8. A compound according to claim 1 wherein B is C₁-C₆ alkyl, C₁-C₆ haloalkyl, cycloalkyl, cycloalkylalkyl, heterocycloalkylalkyl, aralkyl, heteroarylalkyl, or heterocycloalkyl.

9. A compound according to claim 1 wherein B is n-Bu, t-Bu, Me, CF₃, 2,2-dimethylpropyl, 3,3,3-trifluoropropyl, cyclohexyl, cyclopentyl, cyclobutyl, cyclopropyl, cyclohexylmethyl, benzyl, 4-fluorobenzyl, 3,4-dichlorobenzyl, alpha-methylbenzyl, piperidinyl, or tetrahydropyranyl.

10. A compound according to claim 1 wherein B is unsubstituted or substituted aryl.

11. A compound according to claim 1 wherein
B is phenyl unsubstituted or substituted with one or more groups selected from halogen, CN, —NO₂, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, heterocycloalkylalkyl, COR⁴, CO₂R⁴, and SO₂R⁴; or
B is phenyl substituted with substituted or unsubstituted aryl, cycloalkyl, heterocycloalkyl or heteroaryl.

12. A compound according to claim 1 wherein
B is heteroaryl unsubstituted or substituted with one or more groups selected from halogen, CN, —NO₂, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, heterocycloalkylalkyl, COR⁴, CO₂R⁴, and SO₂R⁴; or
B is heteroaryl substituted with substituted or unsubstituted aryl, cycloalkyl, heterocycloalkyl or heteroaryl.

13. A compound according to claim 1 wherein B is phenyl substituted with one or more groups selected from Me, Et, i-Pr, n-Bu, t-Bu, F, Cl, CF₃, OMe, OEt, OCF₃, OCHF₂, CN, —NO₂, CO₂Me, NHAc, NH₂, NMe₂, COMe, NHSO₂Me, NHSO₂Et, and NHSO₂—(CH₂)₄-Me.

14. A compound according to a claim 1 wherein B is phenyl substituted with piperazin-1-yl, N-methylpiperazin-1-yl, N-isopropylpiperazin-1-yl, morpholin-1-yl, piperidin-1-yl, pyrrolidin-1-yl, or morpholin-1-ylmethyl.

15. A compound according to claim 1 wherein B is substituted or unsubstituted heteroaryl.

16. A compound according to any one of claims 1-3 wherein the compound is according to formulae Ig, or IIg; and the group B—Y— is selected from 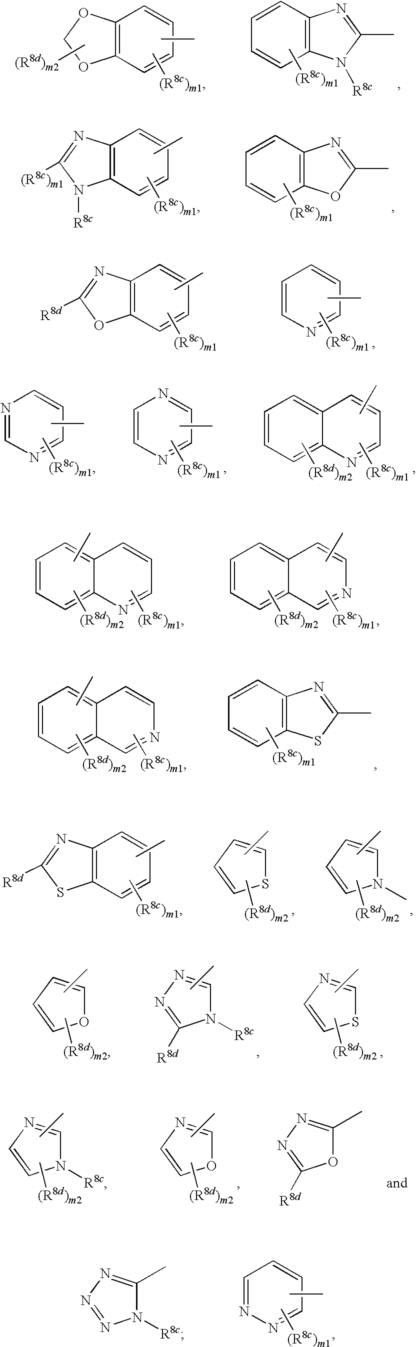
wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, COR⁴, CO₂R⁴, and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl; and each of subscript m1 and m2 is independently selected from 0, 1, and 2.

17. A compound according to claim 16 wherein each of R^{8c} or R^{8d} is selected from H, halogen, C₁-C₆ alkyl, C₁-C₆ haloalkyl, heterocycloalkylalkyl, cycloalkyl, heterocycloalkyl, heterocycloalkylphenyl, aryl or heteroaryl.

18. A compound according to any one of claims 1-3 wherein the compound is according to formulae Ig, or IIg; and the group B—Y— is selected from

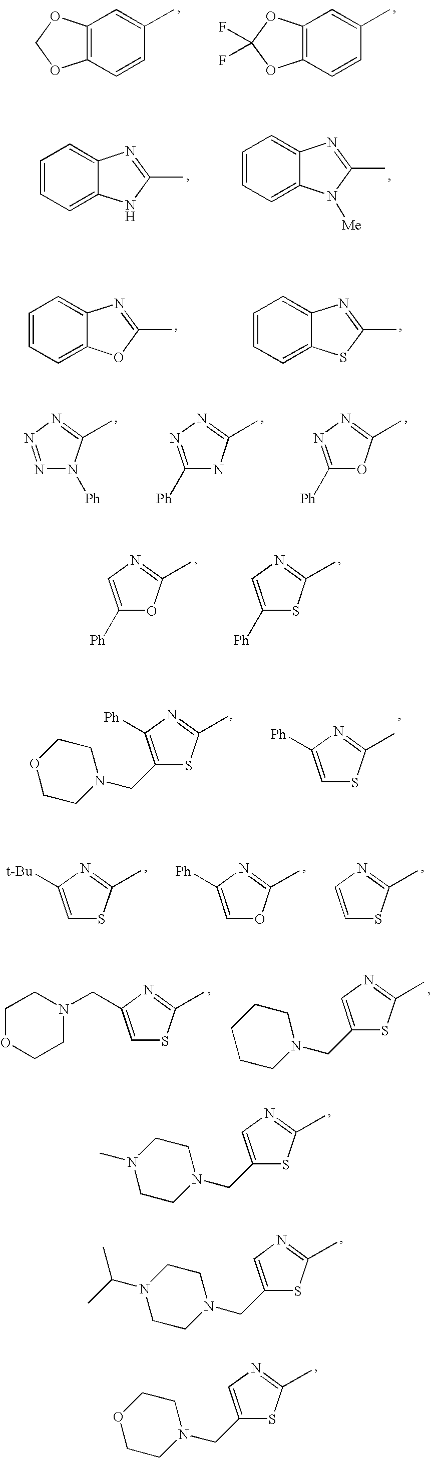

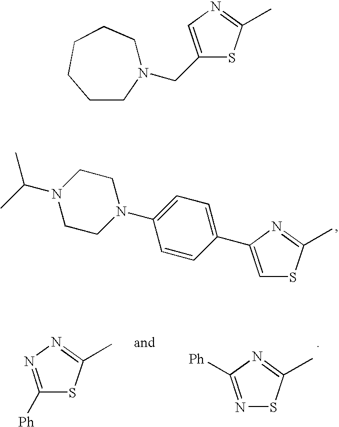

19. A compound according to claim 1 wherein the compound is according to formulae IVa, IVb, IVc or IVd: 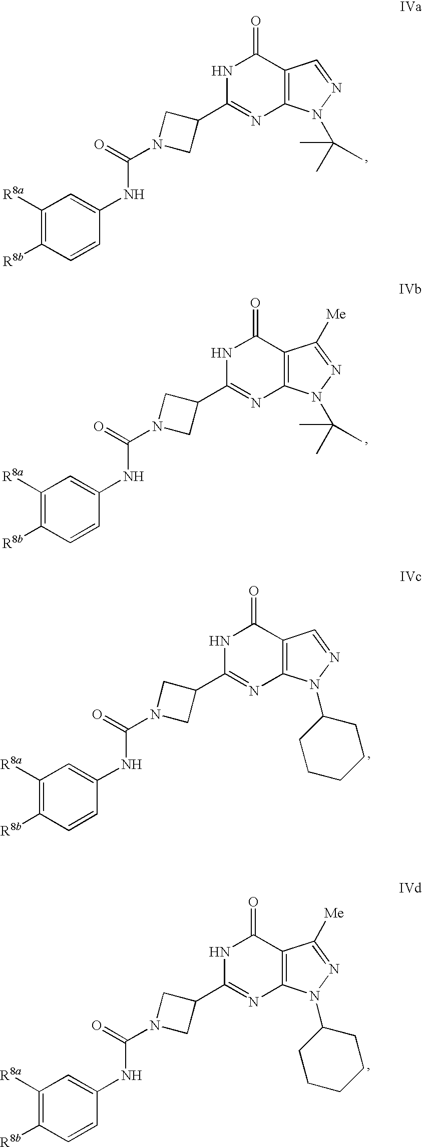
wherein R^{8a} and R^{8b} are independently selected from H, halogen, CN, —NO₂, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, SO₂R⁴, heterocycloalkylalkyl, cycloalkyl, heterocycloalkyl.

20. A compound according to claim 19 wherein each of R^{8a} or R^{8b} is selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A compound according to claim 1 wherein the compound is according to formulae VIIIa, VIIIb, VIIIc or VIIId: 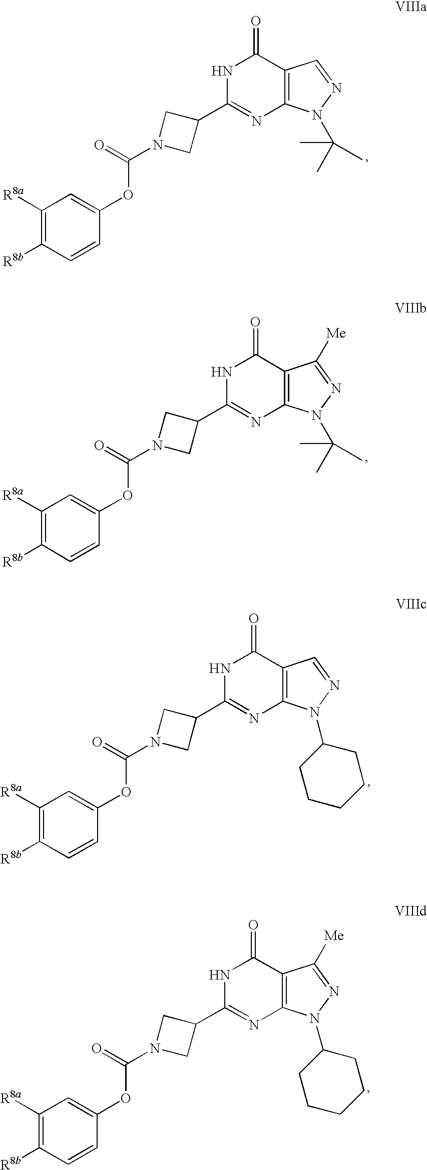
wherein R^{8a} and R^{8b} are independently selected from H, halogen, CN, —NO₂, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, COR⁴, CO₂R⁴, SO₂R⁴, cycloalkyl, heterocycloalkyl, heterocycloalkylalkyl heterocycloalkylphenyl, aryl or heteroaryl.

34. A compound according to claim 33 wherein R^{8a} and R^{8b} are independently selected from H, C₁-C₆ alkyl, halo, CN, C₁-C₆ alkoxy, C₁-C₆ haloalkyl, C₁-C₆ haloalkoxy, amino, dialkylamino, heterocycloalkyl, and heterocycloalkylalkyl.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. A compound according to claim 1 wherein the compound is according to formulae XXa, XXb, XXc or XXd: 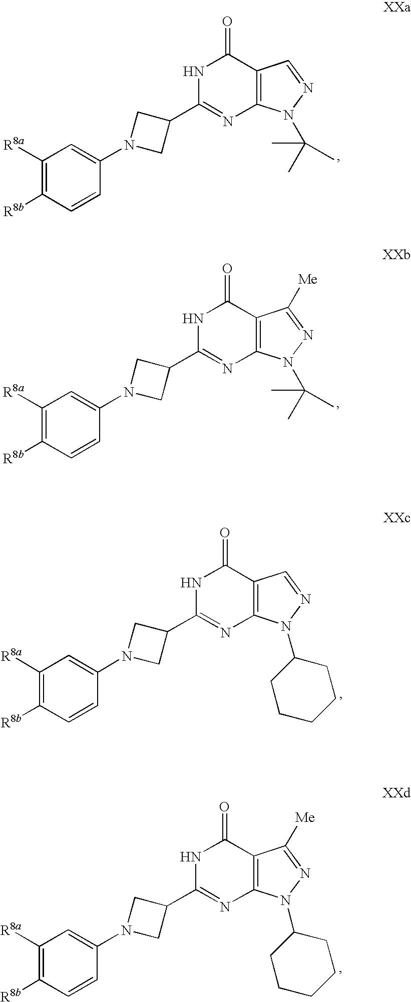
wherein R^{8a} and R^{8b} are independently selected from H, CN, —NO₂, NR⁴R⁵, NR⁵COR⁴, CONR⁴R⁵, NR⁵SO₂R⁴, SO₂NR⁵R⁴, C₁-C₆ alkyl, C₁-C₆ haloalkyl, C₁-C₆ alkoxy, C₁-C₆ haloalkoxy, substituted or unsubstituted cycloalkyl, substituted or unsubstituted cycloalkylalkyl, substituted or unsubstituted heterocycloalkyl, substituted or unsubstituted heterocycloalkylalkyl, substituted or unsubstituted heteroaryl, substituted or unsubstituted aralkyl, COR⁴, CO₂R⁴, and SO₂R⁴.

65. A compound according to claim 64 wherein R^{8a} and R^{8b} are independently selected from H, C₁-C₆ alkyl, halo, CN, C₁-C₆ alkoxy, C₁-C₆ haloalkyl, C₁-C₆haloalkoxy, amino, dialkylamino, heterocycloalkyl, and heterocycloalkylalkyl.

66. (canceled)

67. (canceled)

68. A compound according to claim 64 wherein R^{8a} is H, Me, NMe₂, Cl or F; and R^{8b} is H, Me, Cl or F.

69. A compound according to claim 64 wherein R^{8a} is H; and R^{8b} is H, Me, Cl, F, NMe₂, OMe, i-Pr, t-Bu, OCF₃, CF₃, CN, morpholin-1-yl, piperazin-1-yl, N-methylpiperazin-1-yl or N-isopropylpiperazin-1-yl.

70. (canceled)

71. (canceled)

72. A compound according to claim 1 wherein the compound is according to formulae XXIVa, XXIVb, XXIVc or XXIVd: 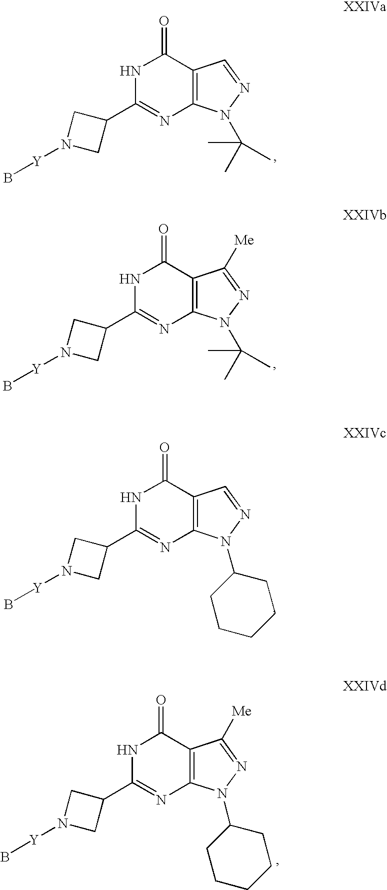
wherein Y is substituted or unsubstituted heteroaryl; and B is selected from H, C₁-C₆ alkyl, C₁-C₆ haloalkyl, halo, —CN, NO₂, aryl, heteroaryl, cycloalkyl, cycloalkylalkyl, heterocycloalkyl, heterocycloalkylalkyl, heterocycloalkylphenyl, and aralkyl.

73. (canceled)

74. A compound according to claim 72 wherein B is selected from pyridyl, pyrimidyl, pyrazinyl, quinolinyl, isoquinolinyl, benzimidazolyl, benzoxazolyl, benzthiazolyl, benz[1,3]dioxalyl, thiophenyl, pyrrolidinyl, furanyl, triazolyl, thiazolyl, imidazolyl, oxazolyl, oxadiazolyl, and tetrazolyl.

75. A compound according to claim 72 wherein B is selected from H, Me, t-Bu, F, Cl, CF₃, —CN, NO₂, Ph, morpholin-1-yl, piperidin-1-yl, piperazin-1-yl, N-Me-piperazin-1-yl, N-i-Pr-piperazin-1-yl, morpholinylmethyl, piperidinylmethyl, piperazinylmethyl, N-Me-piperazinylmethyl, N-i-Pr-piperazinylmethyl, morpholinylphenyl, piperidinylphenyl, piperazinylphenyl, N-Me-piperazinylphenyl, and N-i-Pr-piperazinylphenyl.

76. A compound according to claim 72 wherein the group B—Y— is selected from 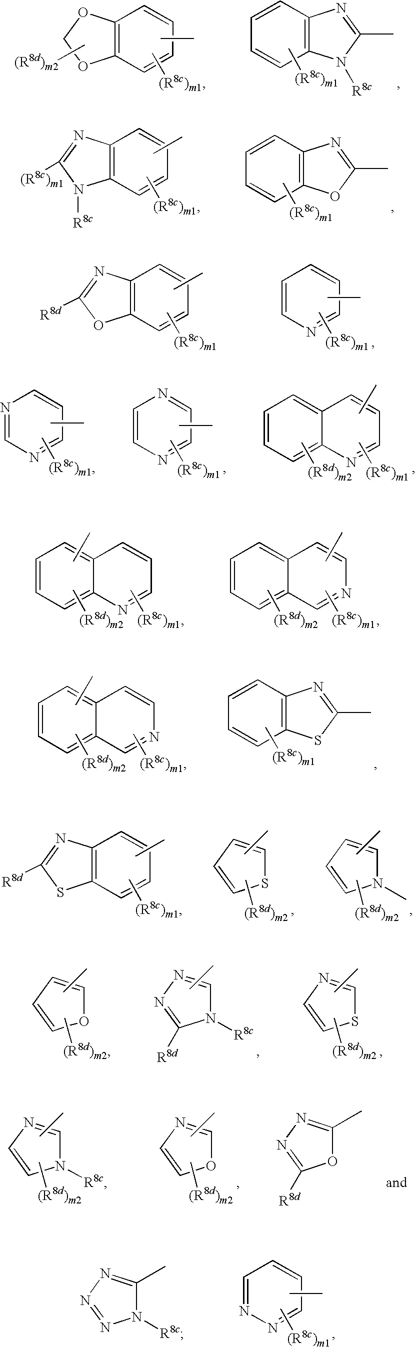
wherein each one of R^{8c} or R^{8d} is independently selected from H, halogen, CN, —NO₂, NR⁴R⁵, NR⁵COR⁴, CONR⁴R⁵, NR⁵SO₂R⁴, SO₂NR⁵R⁴, SO₂R⁴, COR⁴, CO₂R⁴, and SO₂R⁴, or
each one of R^{8c} or R^{8d} is independently selected from C₁-C₆ alkyl, C₁-C₆ haloalkyl, C₁-C₆ alkoxy, and C₁-C₆ haloalkoxy, or
each one of R^{8c} or R^{8d} is independently selected from heterocycloalkylalkyl, cycloalkyl, heterocycloalkyl, heterocycloalkylphenyl, aryl and heteroaryl;
R^{8e} is selected from H, C₁-C₆ alkyl, and C₁-C₆ haloalkyl; or
R^{8e} is selected from heterocycloalkylalkyl, cycloalkyl, heterocycloalkyl, heterocycloalkylphenyl, aryl or heteroaryl; and
each of subscript m1 and m2 is independently selected from 0, 1, and 2.

77. A compound according to claim 76 wherein each of R^{8c} or R^{8d} is independently selected from H, Me, t-Bu, F, Cl, CF₃, Ph, —CN, NO₂, morpholin-1-yl, piperidin-1-yl, piperazin-1-yl, N-Me-piperazin-1-yl, N-i-Pr-piperazin-1-yl, morpholinylmethyl, piperidinylmethyl, piperazinylmethyl, N-Me-piperazinylmethyl, N-i-Pr-piperazinylmethyl, morpholinylphenyl, piperidinylphenyl, piperazinylphenyl, N-Me-piperazinylphenyl, and N-i-Pr-piperazinylphenyl; and each of subscript m1 and m2 is independently selected from 1 and 2.

78. A compound according to claim 72 wherein the group B—Y— is selected from

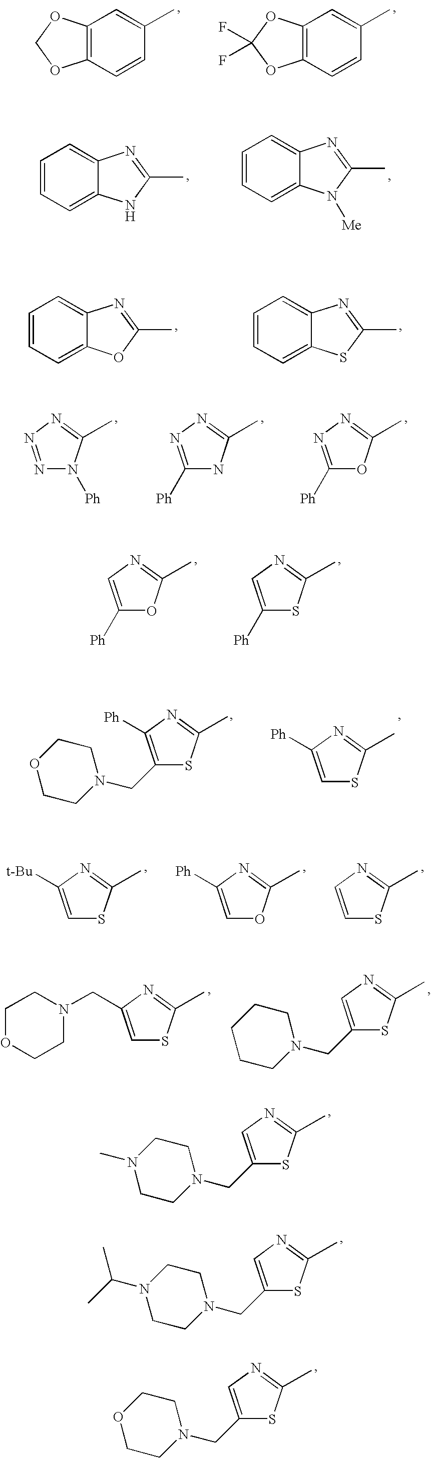

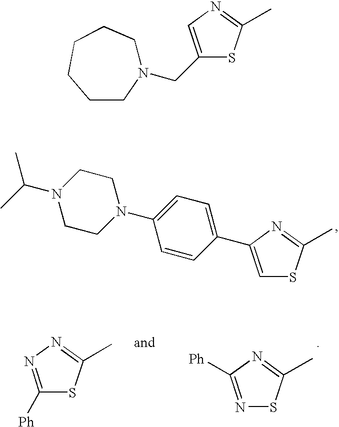

79. (canceled)

80. (canceled)

81. (canceled)

82. (canceled)

83. A compound according to claim 1 wherein the compound is according to formulae XXVIIIa, XXVIIIb, XXVIIIc or XXVIIId: 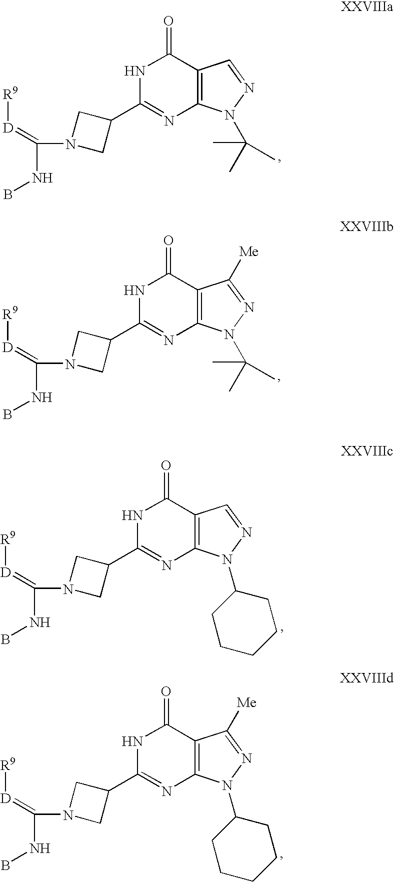
wherein B is selected from C₁-C₆ alkyl, C₁-C₆ haloalkyl, C₁-C₆ alkoxy, C₁-C₆ haloalkoxy, substituted or unsubstituted cycloalkyl, substituted or unsubstituted cycloalkylalkyl, substituted or unsubstituted heterocycloalkyl, substituted or unsubstituted heterocycloalkylalkyl, substituted or unsubstituted heteroaryl and substituted or unsubstituted aralkyl; and the group D-R⁹ is N—CN or CH—NO₂.

84. (canceled)

85. A compound according to claim 83 wherein B is selected from t-Bu, i-Pr, n-Bu, cyclohexyl, cyclopentyl, cyclohexylmethyl, cyclopentylmethyl, piperidinyl, and benzyl.

86. (canceled)

87. (canceled)

88. A compound according to claim 1 wherein the compound is selected from:
N-(benzo[d][1,3]dioxol-5-yl)-3-(1-tert-butyl-3-methyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(4-isopropylpiperazin-1-yl)phenyl)azetidine-1-carboxamide;
3-(1-cyclohexyl-4-oxo-4,5-dihydro-1H-pyrazolo[3,4-d]pyrimidin-6-yl)-N-(4-(4-methylpiperazin-1-yl)phenyl)azetidine-1-carboxamide;
3-(1-cyclohexyl-4-oxo-4,5-dihydro-1H-pyrazolo[3,4-d]pyrimidin-6-yl)-N-(3,4-dimethylphenyl)azetidine-1-carboxamide;
3-(1-cyclohexyl-4-oxo-4,5-dihydro-1H-pyrazolo[3,4-d]pyrimidin-6-yl)-N-(cyclohexylmethyl)azetidine-1-carboxamide;
3-(1-tert-butyl-3-methyl-4-oxo-4,5-dihydro-1H-pyrazolo[3,4-d]pyrimidin-6-yl)-N-phenylazetidine-1-carboxamide;
3-(1-cyclohexyl-4-oxo-4,5-dihydro-1H-pyrazolo[3,4-d]pyrimidin-6-yl)-N-(3-(dimethylamino)phenyl)azetidine-1-carboxamide;
N-(3-chlorophenyl)-3-(1-cyclohexyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(dimethylamino)phenyl)azetidine-1-carboxamide;
3-(1-cyclohexyl-4-oxo-4,5-dihydro-1H-pyrazolo[3,4-d]pyrimidin-6-yl)-N-(4-morpholinophenyl)azetidine-1-carboxamide;
3-(1-cyclohexyl-4-oxo-4,5-dihydro-1H-pyrazolo[3,4-d]pyrimidin-6-yl)-N-phenylazetidine-1-carboxamide;
3-(1-tert-butyl-3-methyl-4-oxo-4,5-dihydro-1H-pyrazolo[3,4-d]pyrimidin-6-yl)-N-(4-fluorophenyl)azetidine-1-carboxamide;
3-(1-cyclohexyl-4-oxo-4,5-dihydro-1H-pyrazolo[3,4-d]pyrimidin-6-yl)-N-(4-methoxyphenyl)azetidine-1-carboxamide;
3-(1-cyclohexyl-3-methyl-4-oxo-4,5-dihydro-1H-pyrazolo[3,4-d]pyrimidin-6-yl)-N-phenylazetidine-1-carboxamide;
3-(1-tert-butyl-3-methyl-4-oxo-4,5-dihydro-1H-pyrazolo[3,4-d]pyrimidin-6-yl)-N-cyclohexylazetidine-1-carboxamide;
3-(1-tert-butyl-4-oxo-4,5-dihydro-1H-pyrazolo[3,4-d]pyrimidin-6-yl)-N-(4-fluorophenyl)azetidine-1-carboxamide;
3-(1-tert-butyl-3-methyl-4-oxo-4,5-dihydro-1H-pyrazolo[3,4-d]pyrimidin-6-yl)-N-(4-fluorobenzyl)azetidine-1-carboxamide;
3-(1-cyclohexyl-4-oxo-4,5-dihydro-1H-pyrazolo[3,4-d]pyrimidin-6-yl)-N-(4-isopropylphenyl)azetidine-1-carboxamide;
3-(1-cyclohexyl-4-oxo-4,5-dihydro-1H-pyrazolo[3,4-d]pyrimidin-6-yl)-N-(4-fluorophenyl)azetidine-1-carboxamide;
3-(1-cyclohexyl-4-oxo-4,5-dihydro-1H-pyrazolo[3,4-d]pyrimidin-6-yl)-N-(3,4-difluorophenyl)azetidine-1-carboxamide;
3-(1-cyclohexyl-3-methyl-4-oxo-4,5-dihydro-1H-pyrazolo[3,4-d]pyrimidin-6-yl)-N-(4-fluorophenyl)azetidine-1-carboxamide;
N-benzyl-3-(1-tert-butyl-3-methyl-4-oxo-4,5-dihydro-1H-pyrazolo[3,4-d]pyrimidin-6-yl)azetidine-1-carboxamide;
N-cyclohexyl-3-(1-cyclohexyl-4-oxo-4,5-dihydro-1H-pyrazolo[3,4-d]pyrimidin-6-yl)azetidine-1-carboxamide;
N-benzyl-3-(1-cyclohexyl-3-methyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(trifluoromethoxy)phenyl)azetidine-1-carboxamide;
tert-butyl 3-(3-(1-cyclohexyl-4-oxo-4,5-dihydro-1H-pyrazolo[3,4-d]pyrimidin-6-yl)azetidine-1-carboxamido)piperidine-1-carboxylate;
3-(1-cyclohexyl-3-methyl-4-oxo-4,5-dihydro-1H-pyrazolo[3,4-d]pyrimidin-6-yl)-N-(4-fluorobenzyl)azetidine-1-carboxamide;
3-(1-cyclohexyl-3-methyl-4-oxo-4,5-dihydro-1H-pyrazolo[3,4-d]pyrimidin-6-yl)-N-(4-methoxybenzyl)azetidine-1-carboxamide;
N-(2-(difluoromethoxy)phenyl)-3-(1-(4-fluorophenyl)-3-methyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-fluorobenzyl)azetidine-1-carboxamide;
N-benzyl-3-(1-cyclohexyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-cyclopentylazetidine-1-carboxamide;
N-(4-cyanophenyl)-3-(1-cyclohexyl-4-oxo-4,5-dihydro-1H-pyrazolo[3,4-d]pyrimidin-6-yl)azetidine-1-carboxamide;
N-butyl-3-(1-cyclohexyl-4-oxo-4,5-dihydro-1H-pyrazolo[3,4-d]pyrimidin-6-yl)azetidine-1-carboxamide;
3-(1-cyclohexyl-4-oxo-4,5-dihydro-1H-pyrazolo[3,4-d]pyrimidin-6-yl)-N-(4-(trifluoromethyl)phenyl)azetidine-1-carboxamide;
3-(1-cyclohexyl-4-oxo-4,5-dihydro-1H-pyrazolo[3,4-d]pyrimidin-6-yl)-N-(3,4-dichlorobenzyl)azetidine-1-carboxamide;
N-tert-butyl-3-(1-cyclohexyl-4-oxo-4,5-dihydro-1H-pyrazolo[3,4-d]pyrimidin-6-yl)azetidine-1-carboxamide;
3-(1-cyclohexyl-3-methyl-4-oxo-4,5-dihydro-1H-pyrazolo[3,4-d]pyrimidin-6-yl)-N-(3,4-dichlorobenzyl)azetidine-1-carboxamide;
3-(1-cyclohexyl-4-oxo-4,5-dihydro-1H-pyrazolo[3,4-d]pyrimidin-6-yl)-N-(1-(methylsulfonyl)piperidin-4-yl)azetidine-1-carboxamide;
tert-butyl 4-(3-(1-cyclohexyl-4-oxo-4,5-dihydro-1H-pyrazolo[3,4-d]pyrimidin-6-yl)azetidine-1-carboxamido)piperidine-1-carboxylate;
(S)-3-(1-cyclohexyl-3-methyl-4-oxo-4,5-dihydro-1H-pyrazolo[3,4-d]pyrimidin-6-yl)-N-(1-phenylethyl)azetidine-1-carboxamide; and
3-(1-cyclohexyl-4-oxo-4,5-dihydro-1H-pyrazolo[3,4-d]pyrimidin-6-yl)-N-(3,3,3-trifluoropropyl)azetidine-1-carboxamide;
or a pharmaceutically acceptable salt thereof, and isotopic variants thereof, stereoisomers and tautomers thereof.

89. (canceled)

90. A pharmaceutical composition comprising a pharmaceutically acceptable carrier and a pharmaceutically effective amount of a compound according to claim 1.

91. (canceled)

92. (canceled)

93. (canceled)

94. (canceled)

95. (canceled)

96. (canceled)

97. A method of treatment or prevention of diseases associated with bone and/or cartilage degradation, which comprises administering to a subject in need thereof, a therapeutically effective amount of a compound according to claim 1.

98. A method of treatment or prevention of rheumatoid arthritis, which comprises administering to a subject in need thereof, a therapeutically effective amount of a compound according to claim 1.

99. A method of treatment or prevention of osteoarthritis, which comprises administering to a subject in need thereof, a therapeutically effective amount of a compound according to claim 1.
